(19) United States
(12) Patent Application Publication
(10) Pub. No.: US 2023/0346847 A1
(43) Pub. Date: Nov. 2, 2023

(54) JUVENILE PROTECTIVE FACTORS TO ARREST AND REVERSE AGING IN THE ENTERIC NERVOUS SYSTEM

(71) Applicant: THE JOHNS HOPKINS UNIVERSITY, Baltimore, MD (US)

(72) Inventors: Subhash Kulkarni, Baltimore, MD (US); Pankaj J. Pasricha, Ellicott City, MD (US)

(21) Appl. No.: 18/042,912

(22) PCT Filed: Aug. 24, 2021
(86) PCT No.: PCT/US21/47354

§ 371 Date: Feb. 24, 2023

Related U.S. Application Data
(60) Provisional application No. 63/069,669, filed on Aug. 24, 2020.

(51) Int. Cl. A61K 35/30; A61K 45/06; A61P 1/04

(52) U.S. Cl. CPC A61K 35/30; A61K 45/06; A61P 1/04

(57) ABSTRACT

Compositions and methods of use are provided in the treatment of age-associated gastrointestinal dysmotility disorders, along with other pathological manifestations of intestinal dysfunction and dysmotility disorders.

### CROSS-REFERENCE TO RELATED APPLICATION

This application claims the benefit of U.S. Provisional Application 63/069,669, filed Aug. 24, 2020. The entire contents of this application is incorporated herein by reference in its entirety.

### STATEMENT REGARDING FEDERALLY SPONSORED RESEARCH

This invention was made with government support under grant DK089502 and DK080920 awarded by the National Institutes of Health. The government has certain rights in the invention.

### FIELD OF THE DISCLOSURE

The disclosure relates to compositions and use of these compositions in the treatment of age-associated gastrointestinal dysmotility disorders, along with other pathological manifestations of intestinal dysmotility disorders

### BACKGROUND

The enteric nervous system (ENS) is critical for normal gastrointestinal (GI) function. It is the largest and most diverse component of the autonomic nervous system with cells expressing more than 30 different neurotransmitters and with neuron numbers surpassing those in the spinal cord. The ENS can function largely independent of inputs from the brain and spinal cord. Defects in ENS development are responsible for a range of human disorders.

The development of the human ENS lineage remains poorly understood due to the lack of a suitable model system and the limited access to primary tissue. The ENS regulates significant functions of the gastrointestinal tract that range from normal intestinal motility, digestion, regulation of barrier functions, and intestinal immunity. With age, the regulation of these functions progressively declines suggesting age-associated degeneration in the functional and structural biology of the ENS. With age, the mammalian ENS does indeed have significant deterioration in its structure with significant loss in various populations of NOS1⁺ enteric neurons. However, the biological basis of this loss was previously unknown. This has stymied the discovery of agents that can reverse aging-associated loss of ENS structure and function.

### SUMMARY

In one aspect, we now provide new therapeutic compositions and methods to provide disease modifying therapy for gastrointestinal disorders related to age and other disease associated structural alterations and neurodegeneration in the enteric nervous system.

Accordingly, in certain embodiments, methods of treating a subject with a gastrointestinal disorder are provided, comprises administering to the subject a therapeutically effective amount of an agonist of RET receptor signaling and/or an antagonist of MET receptor signaling, thereby treating the subject. In this and other embodiments, the therapeutically effective amount of the agonist of RET receptor signaling preferably increases the number of neural crest (NC)-derived enteric neuron (NENs) cells relative to Mesodermal-lineage of enteric neuron (MENs) cells. In this and other embodiments, the antagonist of MET receptor signaling preferably decreases the number of MEN cells relative to NENs.

In certain embodiments, an NEN cell comprises one or more markers comprising: MHCST⁻ MET⁻, Ret, Uchl1, Ncam1, Nos1, Plp1, S100b, RET, Sox10, Snap25 and combinations thereof.

In certain embodiments, a MEN cell comprises one or more markers comprising: MHCST⁺ Calcb (CGRP), Met, Cdh3, Slpi, Aebp1, Clic3, Fmo2, Smo, Myl7, Slc17a9, Ntf3, I118 and combinations thereof. In certain embodiments, the MENs co-express of Calcb (CGRP), Met, and Cdh3 genes.

In certain embodiments, an agonist of RET signaling comprises: glial derived neurotrophic factor (GDNF), GDNF analogs, juvenile protective factors (JPF), small molecules, peptide, oligonucleotides, antibodies, antibody fragments, single chain antibodies, antibody mimetics, peptoids, aptamers; enzymes; hormones; organic or inorganic molecules; and/or natural or synthetic compounds.

In certain embodiments, a method of treating a treating a subject with a gastrointestinal disorder is provided and comprises: administering to the subject a therapeutically effective amount of neural crest (NC)-derived enteric neuron (NENs) thereby treating the subject. In this and other embodiments, the NENs are derived from neural crest and MENs are derived from mesoderm. In this and other embodiments, the NENs are identified as being MHCST⁻ MET⁻. In certain embodiments, the isolated NENs are cultured in a medium comprising at least one agonist of RET receptor signaling. In certain embodiments, an agonist of RET signaling is GDNF, a GDNF analog or the combination thereof. In certain embodiments, the NENs are cultured and expanded prior to administering to the subject. In certain embodiments, the method further comprises administering one or more agonists of RET signaling, one or more antagonist of MET receptor signaling or combinations thereof.

In certain embodiments, an antagonist of MET receptor signaling comprises an inhibitor of hepatocyte growth factor (HGF), HGF analogs, small molecules, peptide, oligonucleotides, antibodies, antibody fragments, single chain antibodies, antibody mimetics, peptoids, aptamers; enzymes, hormones, organic or inorganic molecules, natural or synthetic compounds.

In certain embodiments, a method of identifying an agonist of RET receptor signaling, comprises contacting a cell expressing an RET receptor or fragments thereof, with a candidate agent and assaying for one or more activities comprising: detectable labels, cell proliferation, cell maturation, biomarker expression or combinations thereof. In this and other embodiments, the candidate agent increases RET receptor signaling as compared to a normal control. In this and other embodiments, the candidate agent increases neural crest (NC)-derived enteric neuron (NENs) biomarker expression as compared to a normal control.

In certain embodiments, a pharmaceutical composition comprises a therapeutically effective amount of one or more agonists of RET signaling. Exemplary therapeutically effective doses of an agonist of RET signaling include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight. Exemplary effective daily doses of one or more agonists of RET signaling include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

In certain embodiments, a method of treating a subject with a gastrointestinal disorder, age or other disease associated with inflammatory bowel disease and associated intestinal dysmotility: comprises administering to the subject a therapeutically effective amount of an antagonist of RET receptor signaling and/or an agonist of MET receptor signaling, thereby treating the subject. In certain embodiments, the therapeutically effective amount of the antagonist of RET receptor signaling decreases the number of neural crest (NC)-derived enteric neuron (NENs) cells relative to Mesodermal-lineage of enteric neuron (MENs) cells. In certain embodiments, an agonist of MET receptor signaling increases the number of MEN cells relative to NENs. In certain embodiments, an antagonist of RET signaling comprises: RET inhibitors, GDNF inhibitors, small molecules, peptide, oligonucleotides, antibodies, antibody fragments, single chain antibodies, antibody mimetics, peptoids, aptamers; enzymes, hormones, organic or inorganic molecules, natural or synthetic compounds. In certain embodiments, an NEN cell comprises one or more markers comprising: MHCST⁻ MET⁻, Ret, Uchl1, Ncam1, Nos1, Plp1, S100b, RET, Sox10, Snap25 and combinations thereof. In certain embodiments, a MEN cell comprises one or more markers comprising: MHCST⁺, Calcb (CGRP), Met, Cdh3, Slpi, Aebp1, Clic3, Fmo2, Smo, Myl7, Slc17a9, Ntf3, Il18 and combinations thereof. In certain embodiments, the MENs co-express of Calcb (CGRP), Met, and Cdh3 genes.

In certain embodiments, method of treating a treating a subject with a gastrointestinal disorder, comprises: administering to the subject a therapeutically effective amount of mesoderm-derived enteric neuron (MENs) cells, thereby treating the subject. In certain embodiments, the isolated MENs are cultured in a medium comprising at least one agonist of MET receptor signaling.

In certain embodiments, an agonist of RET signaling is administered daily, e.g., every 24 hours. Or, the agonist of RET signaling is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Alternatively, an agonist of RET signaling is administered about once per week, e.g., about once every 7 days. Or, an agonist of RET signaling is administered twice per week, three times per week, four times per week, five times per week, six times per week, or seven times per week. Exemplary effective weekly doses of an agonist of RET signaling include between 0.0001 mg/kg and 100 mg/kg body weight, e.g., 0.001, 0.003, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 10, 20, 30, 40, 50, 60, 70, 80, 90, 95, 96, 97, 98, 99 or 100 mg/kg body weight. For example, an effective weekly dose of an agonist of RET signaling can be between 0.1 μg/kg body weight and 400 μg/kg body weight. Alternatively, an agonist of RET signaling is administered at a fixed dose or based on body surface area (i.e., per m²).

In certain embodiments, an antagonist of RET signaling is administered daily, e.g., every 24 hours. Or, the antagonist of RET signaling is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Alternatively, an antagonist of RET signaling is administered about once per week, e.g., about once every 7 days. Or, an agonist of RET signaling is administered twice per week, three times per week, four times per week, five times per week, six times per week, or seven times per week. Exemplary effective weekly doses of an antagonist of RET signaling include between 0.0001 mg/kg and 100 mg/kg body weight, e.g., 0.001, 0.003, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 10, 20, 30, 40, 50, 60, 70, 80, 90, 95, 96, 97, 98, 99 or 100 mg/kg body weight. For example, an effective weekly dose of an agonist of RET signaling can be between 0.1 μg/kg body weight and 400 μg/kg body weight. Alternatively, an agonist of RET signaling is administered at a fixed dose or based on body surface area (i.e., per m²).

In certain embodiments, a pharmaceutical composition comprises a therapeutically effective amount of one or more antagonists of MET receptor signaling. In certain embodiments, an antagonist of MET receptor signaling is administered daily, e.g., every 24 hours. Or, the antagonist of MET receptor signaling is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Alternatively, an antagonist of MET receptor signaling is administered about once per week, e.g., about once every 7 days. Or, an antagonist of MET receptor signaling is administered twice per week, three times per week, four times per week, five times per week, six times per week, or seven times per week. Exemplary effective daily doses of one or more antagonists of MET receptor signaling include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight. Exemplary effective weekly doses of one or more antagonists of MET receptor signaling include between 0.0001 mg/kg and 100 mg/kg body weight, e.g., 0.001, 0.003, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 10, 20, 30, 40, 50, 60, 70, 80, 90, 95, 96, 97, 98, 99 or 100 mg/kg body weight. For example, an effective weekly dose of an antagonist of MET receptor signaling can be between 0.1 μg/kg body weight and 400 μg/kg body weight. Alternatively, an antagonist of MET receptor signaling is administered at a fixed dose or based on body surface area (i.e., per m²).

Alternatively, an agonist of MET receptor signaling is administered about once per week, e.g., about once every 7 days. Or, an agonist of MET receptor signaling is administered twice per week, three times per week, four times per week, five times per week, six times per week, or seven times per week. Exemplary effective daily doses of one or more antagonists of MET receptor signaling include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight. Exemplary effective weekly doses of one or more antagonists of MET receptor signaling include between 0.0001 mg/kg and 100 mg/kg body weight, e.g., 0.001, 0.003, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 10, 20, 30, 40, 50, 60, 70, 80, 90, 95, 96, 97, 98, 99 or 100 mg/kg body weight. For example, an effective weekly dose of an agonist of MET receptor signaling can be between 0.1 μg/kg body weight and 400 μg/kg body weight. Alternatively, an agonist of MET receptor signaling is administered at a fixed dose or based on body surface area (i.e., per m²).

In certain embodiments, a method of inducing neural crest (NC)-derived enteric neuron (NENs) cell production in a subject, comprising administering therapeutically effective amount of an agonist of RET receptor signaling and/or an antagonist of MET receptor signaling, thereby treating the subject. In this and other embodiments, the therapeutically effective amount of the agonist of RET receptor signaling increases the number of neural crest (NC)-derived enteric neuron (NENs) cells relative to Mesodermal-lineage of enteric neuron (MENs) cells. In embodiments, antagonist of MET receptor signaling decreases the number of MEN cells relative to NENs.

In certain embodiments, a method of treating structural alterations and neurodegeneration in the enteric nervous system of a subject, comprising administering therapeutically effective amount of an agonist of RET receptor signaling and/or an antagonist of MET receptor signaling, thereby treating the subject. In certain embodiments, the method further comprises administering to the subject an effective amount of isolated NENs. In certain embodiments, the NENs are isolated, cultured and expanded prior to administering to the subject. In the context of cell therapy, e.g., adoptive cell therapy, mesoderm samples can be isolated from autologous, allogeneic, haplotype matched, haplotype mismatched, haplo-identical, xenogeneic, cell lines or combinations thereof.

In certain embodiments, a method of correcting sex-biased lineage representation in a subject comprises administering to the subject a therapeutically effective amount of an agonist of RET receptor signaling and/or an antagonist of MET receptor signaling, wherein the therapeutically effective amount of the agonist of RET receptor signaling increases the number of neural crest (NC)-derived enteric neuron (NENs) cells relative to Mesodermal-lineage of enteric neuron (MENs) cells, thereby correcting the sex biased lineage representation. In certain embodiments, the subject is female. In certain embodiments, the subject is male. In certain embodiments, the subject is transgender. In certain embodiments, an antagonist of MET receptor signaling decreases the number of MEN cells relative to NENs. In certain embodiments, an agonist of RET signaling comprises: glial derived neurotrophic factor (GDNF), GDNF analogs, small molecules, peptide, oligonucleotides, antibodies, antibody fragments, single chain antibodies, antibody mimetics, peptoids, aptamers; enzymes, hormones, organic or inorganic molecules, natural or synthetic compounds. In certain embodiments, an NEN cell comprises one or more markers comprising: MHCST⁻ MET⁻, Ret, Uchl1, Ncam1, Nos1, Plp1, S100b, RET, Sox10, Snap25 and combinations thereof. In certain embodiments, a MEN cell comprises one or more markers comprising: MHCST⁺, Calcb (CGRP), Met, Cdh3, Slpi, Aebp1, Clic3, Fmo2, Smo, Myl7, Slc17a9, Ntf3, I118 and combinations thereof. In certain embodiments, the MENs co-express of Calcb (CGRP), Met, and Cdh3 genes. In certain embodiments, the subject suffers from one or more gut disorders that are associated with dysmotility.

In certain embodiments, treating an individual in need of adoptive cell therapy, comprises administering to the individual a therapeutically effective amount of a composition of the disclosure. Adoptive cell therapy involves isolating cells from an individual, expanding the cells ex vivo, and infusing the cells back to the patient. For adoptive cell therapy in the context of this disclosure includes using NENs, in therapeutically effective cell doses in the range of about 10⁴ to about 10¹⁰, e.g. about 10⁹ cells are typically infused at any one time. However, this number can be modified depending on the time between doses, regularity of doses, severity of disease, age, sex and the like. Accordingly, in certain embodiments, the method further comprises administering to a subject in need of the therapy one or multiple doses of the cell or population thereof at therapeutically sufficient amounts. Upon administration of the cells into the subject, the cells can proliferate and increase in number as compared to MENs. In certain embodiments pharmaceutical compositions comprising a therapeutically effective amount of an agonist of RET receptor signaling and/or an antagonist of MET receptor signaling can be co-administered. The cells of the presently disclosed subject matter can be administered by any methods known in the art, including, but not limited to, pleural administration, intravenous administration, subcutaneous administration, rectal administration, intranodal administration, intrathecal administration, intrapleural administration, intraperitoneal administration, and direct administration to the intestines.

In certain embodiments, a pharmaceutical composition comprises a therapeutically effective amount of one or more agonists of RET signaling and one or more antagonists of MET receptor signaling. Exemplary effective doses of one or more agonists of RET signaling and one or more antagonists of MET receptor signaling include between 0.01 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In certain embodiments, a pharmaceutical composition comprises a therapeutically effective amount of one or more antagonists of RET signaling and one or more agonists of MET receptor signaling. Exemplary effective doses of one or more antagonists of RET signaling and one or more agonists of MET receptor signaling include between 0.01 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In certain embodiments, a therapeutically effective dose of the one or more agonists of RET signaling and/or one or more antagonists of MET receptor signaling comprises a range from about 0.001 mg up to 500 mg. In certain embodiments, a therapeutically effective dose of the one or more agonists of RET signaling and/or one or more antagonists of MET receptor signaling comprises a range from about 0.01 mg to about 500 mg, or from about 0.1 mg to about 500 mg, or from about 0.2 mg to about 450 mg, or from about 0.3 mg to about 400 mg, or from about 0.3 mg to about 375 mg, or from about 0.4 mg to about 350 mg, or from about 0.5 mg to about 350 mg, or from about 0.6 mg to about 325 mg, or from about 0.7 mg to about 300 mg, or from about 0.7 mg to about 300 mg, or from about 0.8 mg to about 275 mg, or from about 0.9 mg to about 250 mg, or from about 1 mg to about 245 mg, or from about 1 mg to about 240 mg, or from about 1 mg to about 235 mg, or from about 1 mg to about 230 mg, or from about 1.0 mg to about 225 mg, or from about 1 mg to about 220 mg, or from about 1 mg to about 210 mg, or from about 1 mg to about 200 mg, or from about 1 mg to about 175 mg, or from about 1 mg to about 150 mg, or from about 1 mg to about 145 mg or from about 1 mg to about 140 mg, or from about 1 mg to about 135 mg, or from about 1 mg to about 130 mg, or from about 1 mg to about 125 mg, or from about 1 mg to about 120 mg, or from about 1 mg to about 115 mg, or from about 1 mg to about 110 mg, or from about 1 mg to about 100 mg.

In certain embodiments, a therapeutically effective dose of the one or more antagonists of RET signaling and/or one or more agonists of MET receptor signaling comprises a range from about 0.001 mg up to 500 mg. In certain embodiments, a therapeutically effective dose of the one or more antagonists of RET signaling and/or one or more agonists of MET receptor signaling comprises a range from about 0.01 mg to about 500 mg, or from about 0.1 mg to about 500 mg, or from about 0.2 mg to about 450 mg, or from about 0.3 mg to about 400 mg, or from about 0.3 mg to about 375 mg, or from about 0.4 mg to about 350 mg, or from about 0.5 mg to about 350 mg, or from about 0.6 mg to about 325 mg, or from about 0.7 mg to about 300 mg, or from about 0.7 mg to about 300 mg, or from about 0.8 mg to about 275 mg, or from about 0.9 mg to about 250 mg, or from about 1 mg to about 245 mg, or from about 1 mg to about 240 mg, or from about 1 mg to about 235 mg, or from about 1 mg to about 230 mg, or from about 1.0 mg to about 225 mg, or from about 1 mg to about 220 mg, or from about 1 mg to about 210 mg, or from about 1 mg to about 200 mg, or from about 1 mg to about 175 mg, or from about 1 mg to about 150 mg, or from about 1 mg to about 145 mg or from about 1 mg to about 140 mg, or from about 1 mg to about 135 mg, or from about 1 mg to about 130 mg, or from about 1 mg to about 125 mg, or from about 1 mg to about 120 mg, or from about 1 mg to about 115 mg, or from about 1 mg to about 110 mg, or from about 1 mg to about 100 mg.

In certain embodiments, the compositions are administered systemically, intravenously, subcutaneously, intramuscularly, intraperitoneally, intravesically, orally, rectally or by instillation.

Other aspects are discussed infra.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “adoptive cell therapy” as used herein refers to a cell-based immunotherapy that, as used herein, relates to the transfusion of NENs, genetically modified or not, that have been expanded ex vivo prior to the transfusion.

As used herein, the term “agent” or “candidate therapeutic agent” is meant to encompass any molecule, chemical entity, composition, drug, therapeutic agent, chemotherapeutic agent, or biological agent capable of modulating receptor signaling, via e.g. MET receptors, RET receptors. The term includes small molecule compounds, antisense oligonucleotides, siRNA reagents, antibodies, antibody fragments bearing epitope recognition sites, such as Fab, Fab′, F(ab′)2 fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

As used herein, the term “agonist”, refers to agents that increase, induce, stimulate, activate, facilitate, or enhance activation the signaling function of the molecule or pathway, e.g., RET signaling.

As used herein, the term “antagonist”, refers to an agent (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any agent that changes any activity of a named protein (signaling molecule, any molecule involved with the named signaling molecule e.g. MET receptor, a named associated molecule, such as a hepatocyte growth factor (HGF) (e.g., including, but not limited to, the signaling molecules described herein). Antagonists are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An antagonist can be a “direct antagonist” that inhibits a signaling target or a signaling target pathway by actually contacting the signaling target.

The term “assay” used herein, whether in the singular or plural shall not be misconstrued or limited as being directed to only one assay with specific steps but shall also include, without limitation any further steps, materials, various iterations, alternatives etc., that can also be used. Thus, if the term “assay” is used in the singular, it is merely for illustrative purposes.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “effective dose” or “effective dosage” is defined as an amount sufficient to achieve or at least partially achieve the desired effect.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the term “marker” or “biomarker” refers to a gene or a protein that identifies a particular cell or cell type. A marker for a cell may not be limited to one marker, markers may refer to a “pattern” or “profile” of markers such that a designated group of markers may identify a cell or cell type from another cell or cell type.

As used in this specification and the appended claims, the term “or” is generally employed in its sense including “and/or” unless the content clearly dictates otherwise.

The term “patient” includes human and other mammalian subjects that receive either prophylactic or therapeutic treatment.

The term “therapeutically effective amount” refers to an amount of a therapeutic or prophylactic agent, such as a biologic agent, that, when incorporated into and/or onto the self-assembled gel composition, produces some desired effect at a reasonable benefit/risk ratio applicable to any treatment. The effective amount may vary depending on such factors as the disease, disorder or condition being treated, the particular formulation being administered, the size of the subject, or the severity of the disease, disorder or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C demonstrate that half of all adult small intestinal myenteric neurons are derived from a non-neural crest lineage. FIG. 1A: Enteric glia, labeled with GFAP (green) in the myenteric plexus from an adult Wnt1-cre:Rosa26-tdTomato mouse. Inset is magnified in color segregated panels, showing GFAP⁺ glia co-expressing tdTomato (red, red arrows). Nuclei are labeled with DAPI (blue). Scale bar=10 μm. FIG. 1B: Enteric neurons, labeled with HuC/D (green) in the myenteric plexus from an adult Wnt1-cre:Rosa26-tdTomato mouse. Inset is magnified in color segregated panels, showing only a subpopulation of neurons co-express tdTomato (red arrows) while many neurons do not (white arrows). Nuclei are labeled with DAPI (blue). Scale bar=10 μm. FIG. 1C: Quantification of tdTomato⁺ and tdTomato neurons in the myenteric ganglia of P60 Wnt1-cre:Rosa26-tdTomato mice show equal proportions of both neuronal types.

FIGS. 2A-2G demonstrate the mesoderm-lineage-tracing and marker expression provide evidence of the mesodermal-derivation of half of all adult small intestinal myenteric neurons. FIG. 2A: Small intestinal LM-MP from adult male Tek-cre:Hprt-tdTomato shows the presence of Tek-derived and hence mesoderm-derived tdTomato⁺ (red) HuC/D⁺ (green) neurons (white arrows) and non-mesoderm-derived tdTomato⁻ neurons (green arrows). Vascular cells depict higher fluorescence intensity (yellow arrow) than cells in the ganglia. Nuclei are labeled with DAPI. Scale bar=10 μm. Cardiac and small intestinal tissues from (FIG. 2B) heart and (FIG. 2C) small intestinal LM-MP layer of adult male Mesp1-cre:Rosa26-tdTomato mice shows the presence of variable tdTomato expression in cells of these tissues. While cardiomyocytes in heart and cells within the myenteric ganglia (white arrows) exhibit lower tdTomato fluorescence, similar to the tissues from Tek-cre:Hprt-tdTomato mice in (FIG. 2A), the vascular cells exhibit higher fluorescence intensity (yellow arrows). Scale bar=10 μm. FIG. 2D: Small intestinal LM-MP from adult male Mesp1-cre:Rosa26-tdTomato shows the presence of mesoderm-derived tdTomato⁺ (red) HuC/D⁺ (green) neurons (white arrows) and non-mesoderm-derived tdTomato-neurons (green arrows). Nuclei are labeled with DAPI. Scale bar=10 μm. FIG. 2E: Quantification of tdTomato-expressing and non-expressing neurons in the myenteric ganglia of P60 Mesp1-cre:Rosa26-tdTomato mice. MHCst immunolabeling exclusively marks all the (FIG. 2F) mesoderm-derived adult HuC/D⁺ (green) neurons in the small intestinal LM-MP of Mesp1-cre:Rosa26-tdTomato mice; and (FIG. 2G) all the non-NC-derived neurons in the small intestinal LM-MP of Wnt1-cre:Rosa26-tdTomato mice (white arrows). MHCst does not label tdTomato+NC-derived cells (yellow arrows). Nuclei are stained with DAPI (blue). Scale bar=10 μm.

FIGS. 3A-3H show the cellular and molecular phenotyping of MENs and NENs. MET immunostaining (green) labels (FIG. 3A) Wnt1-cre:Rosa26-tdTomato⁻ MENs and (FIG. 3B) a population of Mesp1-cre:Rosa26-tdTomato⁺ MENs (white arrow) while not labeling all MENs (red arrow). FIG. 3C: RET immunostaining (green) only labels Wnt1-cre:Rosa26-tdTomato⁺ NENs (green arrow) and not tdTomato⁻ MENs (white arrows). FIG. 3D: NOS1 is expressed by both tdTomato⁺ NENs (red, white arrows) and tdTomato⁻ MENs (blue arrows) in an adult Wnt1-cre:Rosa26-tdTomato mouse, but most MENs do not express NOS1 (marked by *). FIG. 3E: NEN lineage contains significantly higher proportions of NOS1⁺ neurons compared to MEN lineage in Wnt1-cre:Rosa26-tdTomato mice. Data represent mean±S.E.M. (**** p<0.0001). FIG. 3F: Both tdTomato⁺ (red) and tdTomato⁻ neurons in the myenteric plexus of an adult Wnt1-cre:Rosa26-tdTomato mouse (HuC/D, green) express CGRP (blue) Inset showing a tdTomato⁻ CGRP⁺ neuron (white arrow) is magnified on the right. FIG. 3G: MEN lineage contains significantly higher proportions of CGRP⁺ neurons compared to NEN lineage in Wnt1-cre:Rosa26-tdTomato male mice. Data represent mean S.E.M. (*** p<0.001). FIG. 3H: MENs express the protein P-Cadherin/CDH-3 (green; white arrows) while tdTomato⁺ (red) NENs do not (blue arrows) in myenteric ganglia from adult Wnt1-cre:Rosa26-tdTomato mice. Nuclei in (FIG. 3B), (FIG. 3C), (FIG. 3H) are labeled with DAPI (blue). Scale bar for all images denotes 10 μm.

FIGS. 4A-4C demonstrate that scRNAseq-analyses identifies the distinct transcriptomic profile of the MENs. FIG. 4A: UMAP representation of 11,123 sequenced high-quality cells that were identified as meeting a 200 UMI minimum threshold with a mitochondrial read ratio of less than 20%. Clusters were annotated by markers that were found to be cell-type specific by searching UniProt, Allen Cell Atlas and Pubmed databases. Cells of the neural crest lineage were then identified as NC-cells by co-expression of neural crest marker genes Ret and Sox10, or as MENs by co-expression of Calcb (CGRP), Cdh3, and Met genes. FIG. 4B: Cluster-specific expression patterns of select markers. FIG. 4C: Validation of the MENs-specific marker genes discovered in the scRNAseq analyses by immunohistochemistry and confocal microscopy of small intestinal LM-MP from adult male Wnt1-cre:Rosa26-tdTomato mice. Immunostaining of the proteins AEBP1, CFTR, CLIC3, FMO2, NT3, SLPI, SMO, MYL7, IL-18, SLC17A9, and CDH3 (green; green arrows) was found to be localized to tdTomato⁻ MENs. tdTomato⁺ (red) NC-cells did not immunostain for these markers (red arrows). Nuclei were labeled with DAPI (blue). Scale bar denotes 10 μm.

FIGS. 5A, 5B demonstrate that SNAP-25 expression is restricted to the neural crest lineage in the adult myenteric ganglia. SNAP-25 expression (green) co-localizes with tdTomato (red) but not with the MENs marker MHCst (cyan) as observed in (FIG. 5A) 2D views and (FIG. 5B) orthogonal views of a myenteric ganglia from a Wnt1-cre:Rosa26-tdTomato mouse that was immunolabeled with antibodies against MHCst and SNAP-25. Nuclei were labeled with DAPI (blue). Scale bar denotes 10 μm.

FIGS. 6A-6K demonstrate that GDNF and HGF signaling regulate age-dependent changes in NENs and MENs proportions. FIGS. 6A, 6B: Immunostaining myenteric plexus tissue from juvenile and mature Wnt1-cre:Rosa26-tdTomato mice with antibodies against the pan-neuronal marker HuC/D (green). FIG. 6C: Age-associated loss of NENs and gain of MENs in the small intestinal LM-MP of maturing and aging Wnt1-cre:Rosa26-tdTomato mice. Data represent mean±S.E.M. (p<0.0001). FIG. 6D: Western blot analyses of GDNF (green) and the house-keeping protein β-actin (red) expression in LM-MP tissues from mice of ages P10, P30, and P90. (n=3 mice per group; each sample is a biological replicate). Fluorescent intensities of the two bands of GDNF (that correspond to ˜25 kD bands of protein marker) were quantified together. The lower band of GDNF is present only in the P10 tissues and disappears in P30 and P90 adult murine tissues. FIG. 6E: Western blot analyses of HGF (green) and the house-keeping protein β-Actin (red) expression in LM-MP tissues from mice of ages P10, P30, and P90. (n=3 mice per group; each sample is a biological replicate). Fluorescent intensities of the two bands of HGF (that are between 50 kD and 37 kD bands of the protein marker) were quantified together. FIG. 6F: The normalized fluorescent intensity of HGF protein to house-keeping protein β-Actin was compared between the three age groups. HGF expression significantly increased from P10 through P90. Data represent mean±S.E.M. (p=0.02). FIG. 6G: Age-dependent increase in Hgf mRNA transcript expression (normalized to the house-keeping gene Hprt) in the myenteric plexuses of P10, P30, and P90 old mice. Data represent mean±S.E.M. (p<0.0001). FIG. 6H: The normalized fluorescent intensity of GDNF protein to house-keeping protein β-Actin compared between the three age groups. GDNF presence was highest in P10 group and was significantly reduced in P30 and P90 groups. Data represent mean±S.E.M. (p=0.0348). FIG. 6I: Percent proportions of tdTomato⁻ MENs and mean HuC/D⁺ neurons/ganglia in LM-MP of cohorts of Wnt1-cre:Rosa26-tdTomato mice that were dosed with HGF or Saline from P10 to P20 age. Data represent mean±S.E.M. (*** p<0.001). FIG. 6J: Percent proportions of tdTomato⁻ MENs and mean HuC/D⁺ neurons/ganglia in LM-MP of cohorts of Wnt1-cre:Rosa26-tdTomato mice that were dosed with GDNF or Saline from P10 to P20 age. Data represent mean±S.E.M. (*** p<0.001). FIG. 6K: Percent proportions of tdTomato⁻ MENs and mean HuC/D⁺ neurons/ganglia in LM-MP of cohorts of Wnt1-cre:Rosa26-tdTomato mice that were dosed with GDNF or Saline from P60 to P70 age. Data represent mean±S.E.M. (*** p<0.001).

FIGS. 7A-7D demonstrate that reduced RET signaling accelerates ENS aging to cause pathology. FIG. 7A: HuC/D immunostaining (green) LM-MP tissues from 16-week-old Ret^{+/CFP} (Ret^{+/-}) mouse shows mutually exclusive expression of Ret-CFP (cyan, green arrow) and MHCst (red, red arrow) MENs. Nuclei are stained with DAPI (blue). Scale bar=10 μm. FIG. 7B: Quantification of Ret-CFP⁺ neurons from 9- and 16-week-old Ret^{+/-} mice show age-associated loss of Ret-CFP⁺ neurons. Data represent mean±S.E.M. (* p<0.05). FIG. 7C: Quantification of MHCst⁺ MENs shows significant increase in their proportions in Ret^{+/-} mice but not in Ret^{+/+} mice with age. Data represent mean S.E.M. (**** p<0.0001). FIG. 7D: Measures of whole gut transit time (WGTT) in cohorts of Ret^{+/-} and Ret^{+/+} mice MENs show significant slowing of whole gut transit of Ret^{+/-} but not Ret^{+/+} mice with age. Data represent mean±S.E.M. (*=p<0.05).

FIGS. 8A-8C demonstrate that GDNF normalizes altered intestinal motility by increasing NENs proportions in the aging gut. FIG. 8A: Measures of whole gut transit time (WGTT) in GDNF (treated with GDNF) and Control (treated with Saline) cohorts of 17-month-old mice taken before the start of treatments and after the end of 10 consecutive days of treatment shows that the two groups are matched in their transit times before treatment, but GDNF treatment significant decreases average transit times when compared to the control cohort. Data represent mean±S.E.M. (***=p<0.001). FIG. 8B: Quantification of percent MHCst⁺ MENs per HuC/D-labeled neurons in myenteric ganglia in the GDNF and Control cohorts shows significant decrease in their proportions in GDNF-treated cohort but not in saline-treated control cohort. Data represent mean±S.E.M. (**** p<0.0001). FIG. 8C: Quantification of numbers of RET NENs per myenteric ganglia shows significant increase in their numbers in GDNF cohort mice when compared with Control cohort mice. Data represent mean±S.E.M. (**** p<0.0001).

FIGS. 9A-9C show the presence of putative MENs and their molecular regulatory mechanisms in the human gut. FIG. 9A: Putative human MENs (red arrows) and NENs (green arrows) identified by presence or absence of the murine MENs markers, MET (blue) and MHCst (red) in subpopulations of HuC/D⁺ (green) small intestinal myenteric neurons from normal human duodenal tissue. FIG. 9B: Non-negative matrix factorization (NMF) was used to decompose our murine scRNAseq data from adult LM-MP tissues into 50 distinct latent spaces (called NMF patterns), of which four NMF patterns were found to be specific to MENs scRNAseq cell cluster. FIG. 9C: MENs-specific NMF patterns 32 and 41 were significantly upregulated in bulk-RNAseq of OD patients compared to controls. Data represent mean±S.E.M.

FIGS. 10A-10C show the presence and absence of tdTomato reporter expression in the myenteric ganglia of adult neural crest lineage-traced mice. Interrogation of three different neural crest-specific lineage-traced mouse lines indicates that only a subpopulation of HuC/D⁺ adult enteric neurons (green) is derived from the neural crest, as seen by its co-expression of tdTomato (red, red arrows), driven by its respective neural crest-specific gene lineage marker, as indicated (FIG. 10A) Wnt1-cre:Rosa26-tdTomato; (FIG. 10B) Wnt1-cre:Hprt-tdTomato; (FIG. 10C) Pax3-cre:Rosa26-tdTomato. HuC/D⁺ neurons (green) that do not express tdTomato (white arrows) are not derived from the neural crest. In addition, the adult small intestinal myenteric ganglia from these lineage-traced mice shows heterogenous fluorescence of floxed tdTomato reporter protein in their myenteric cells. In (FIG. 10A) HuC/D immunostained Wnt1-cre:Rosa26-tdTomato LM-MP tissue as well as (FIG. 10B) HuC/D immunostained Wnt1-cre:Hprt-tdTomato LM-MP tissue, where tdTomato-expression (red) marks neural crest-derived cells, and HuC/D (green) labels myenteric neurons, we observed the presence of higher intensity fluorescence (red arrows) and lower intensity fluorescence (orange arrows) in various tdTomato⁺ neurons. The myenteric ganglia also contained HuC/D⁺ neurons that did not express tdTomato (green arrow) and these neurons alone were deemed to be non-neural crest-derived neurons. Nuclei are labeled with DAPI (blue). Scale bar denotes 10 μm.

FIGS. 11A-11D demonstrate that Tek-expressing and Mesp1-expressing mesodermal lineage contributes to adult enteric neurons. FIG. 11A: Adult small intestinal LM-MP layer from a male Tek-EGFP mouse, where EGFP expression (green) labels Tek-expressing cells was immunostained with specific antibodies against the pan-neuronal marker PGP9.5 (red) and imaged under a confocal microscope. The Tek-EGFP expression did not label any cells within the myenteric plexus marked by PGP9.5 expression showing that adult small intestinal myenteric neurons did not express Tek. Nuclei are labeled with DAPI (blue). Scale bar denotes 10 μm. FIG. 11B: The presence of higher intensity fluorescence (red arrows) and lower intensity fluorescence (green arrow) was observed in various tdTomato⁺ cells in a section of heart tissue from a 6-month-old Mesp1-cre:Rosa26-tdTomato adult male mouse, where tdTomato (red) labels Mesp1-derived cells of the mesodermal lineage that includes cardiomyocytes and vasculature. Nuclei are stained by DAPI (blue). Scale bar denotes 10 μm. FIG. 11C: Adult small intestinal LM-MP layer from a 6-month-old male Mesp1-cre:Rosa26-tdTomato mouse, where tdTomato (red) cells are mesoderm-derived cells, was immunostained with specific antibodies against the pan-neuronal marker HuC/D (green) and imaged under a confocal microscope. tdTomato-expressing cells outside the ganglia (red arrows) were brighter than tdTomato⁺ neurons inside the ganglia (green arrows). Nuclei are labeled with DAPI (blue). Scale bar denotes 10 μm. FIG. 11D: MHCst⁺ (cyan) labels all tdTomato⁺ (red) HuC/D⁺ (green) adult neurons (white arrows) but not the tdTomato⁻ neurons (yellow arrow) in the mesoderm-specific Tek-cre:Rosa26-tdTomato lineage-traced lines. Nuclei are stained with DAPI (blue). Scale bar=10 μm.

FIGS. 12A-12K show the validation of MENs-specific markers by immunohistochemistry and confocal microscopy. Small intestinal LM-MP from several adult 5-month-old male Wnt1-cre:Rosa26-tdTomato mice, where tdTomato-expression (red) labels neural crest-derived cells, was used to ascertain and validate the MENs-specific expression of the novel MENs marker genes. FIG. 12A: MHCst-specific antibody S46 (green) was found to label tdTomato non-expressing cells within the myenteric ganglia as well as cells outside of the myenteric ganglia. Neural crest-derived cells within the myenteric ganglia expressed tdTomato. Nuclei are labeled with DAPI (blue). Scale bar denotes 10 μm. The immunostaining of the MENs-markers elucidated through scRNAseq analyses: (FIG. 12B) CDH3, (FIG. 12C) AEBP1, (FIG. 12D) SLPI, (FIG. 12E) CFTR, (FIG. 12F) NT-3, (FIG. 12G) CLIC3, (FIG. 12H) FMO2, (FIG. 12I) IL-18, (FIG. 12J) MYL-7, and (FIG. 12K) SLC17A9 (all immunolabeled cells in these panels are green; green arrows) was found to be localized to cells without tdTomato expression, while the tdTomato⁺ cells were found not to express these MENs-specific markers (red arrow). Nuclei in these tissues were labeled with DAPI (blue). Scale bar denotes 10 μm.

FIGS. 13A-13F show the scRNAseq metrics. FIG. 13A: MENs are significantly larger in size as compared to the NENs, as the mean Feret's Diameter of the Wnt1-cre:Rosa26-tdTomato⁻ MENs is significantly more than that of the Wnt1-cre:tdTomato⁺ NENs. (mean S.E.M of Feret's Diameter (in μm): MENs (n=143 cells) 17.47±0.36; NENs (n=143 cells) 13.05±0.23; p value<0.0001). FIG. 13B: UMAP representation of sequenced cells generated from 10× Genomics Chromium-derived libraries from the two mouse samples, taken from separate littermate adult male mice, shows similar representation of their cells across the various clusters. FIG. 13C: UMAP representation of sequenced cells shows annotation of the various clusters, with presence of clusters identified as NENs by their co-expression of Ret and Sox10. The MENs cluster was identified by its co-expression of MENs-marker genes Calcb, Cdh3, and Met, discovered in a previous experiment. FIG. 13D: The average UMI per cell in the MEN cluster was found to be significantly more than that in the NENs clusters (mean±S.E.M of UMI: MENs 4045.6±113.3; NENs 1247.9±38.9; p value<2.2e−16). FIG. 13E: Sparklines plot of single cell transcriptomic data from Zeisel et al⁴⁵ of Wnt1-cre:tdTomato⁺ NENs shows that while the MENs-specific genes were only occasionally detected in some classes of NENs, the expression of Snap25 was ubiquitously detected in all NENs. FIG. 13F: Sparklines plot of single cell transcriptomic data presented in our study shows that while Snap25 was expressed in NC-derived cells, its expression was not detected in MENs. Note the similar maximum expression values of Snap25 between NENs in (FIG. 13E) and NC-derived cells in our transcriptomic data.

FIGS. 14A, 14B show the HGF and GDNF ratios in maturing and adult murine gut. FIG. 14A: Ratio of HGF:GDNF Ct values from the small intestinal LM-MP of post-natal mice at different ages during their maturation. FIG. 14B: Ratio of HGF:GDNF expression values from the full thickness small intestinal tissue of adult mice taken from the Tabula muris senis database⁸⁶.

FIGS. 15A-15D demonstrate the effect of GDNF treatment on RET-expressing NENs and MHCst-expressing MENs in aging mice. Representative image of MHCst (red) and HuC/D (green)-immunostained myenteric ganglia of (FIG. 15A). saline-treated control and (FIG. 15B). GDNF-treated 17-month-old mice show MHCst⁺ (yellow arrows) and MHCst neurons. Note the reduction in MHCst-immunostaining on inter-ganglionic fibers in the 17-month-old mice when treated with GDNF, compared to control mice. Nuclei are stained with DAPI (blue). Scale bar indicates 10 μm. Representative image of RET-immunostained (green) myenteric ganglia of (FIG. 15C). saline-treated control and (FIG. 15D). GDNF-treated 17-month-old mice. Nuclei are stained with DAPI (blue). Scale bar indicates 10 μm.

FIGS. 16A-16C demonstrate that the putative mesoderm-derived enteric neurons (MENs) are present in adult human myenteric ganglia. FIGS. 16A, 16B: The mesodermal specific markers, MET and MHCst are expressed by a subpopulation of human enteric neurons. Myenteric ganglia from normal duodenal tissue of 3 different adult human subjects express the pan-neuronal marker HuC/D (green), with distinct subpopulations positive and negative for MET (blue) and MHCst (red). MET and MHCst-expressing neurons (red arrows) in the human ENS are assumed to be mesoderm-derived enteric neurons (MENs) and those not expressing these MENs markers (green arrows) are presumably NENs. Nuclei are labeled with DAPI (gray). Scale bar=10 μm. FIG. 16C: Colonic human myenteric neurons expressing HuC/D (green) contain two populations of neurons that can be differentiated by their expression of MENs marker MHCst (red arrows). Nuclei are labeled with DAPI (gray). Scale bar=10 μm.

FIG. 17 shows the analyses of human plasma proteome from aging individuals using the LonGenity database. Analyses of human plasma proteome from 1,025 individuals was performed by Sathyan et al⁶⁰ and the correlation of various plasma proteomes with aging was calculated. Of the genes that showed significant correlation with age (p<0.05), we found that the HGF levels in human plasma correlated positively with age (above 0), while the levels of GDNF and RET correlated negatively with age (below 0).

FIG. 18 shows the projection of the bulk RNA sequencing data of intestinal tissue from Control and Patients with Obstructed Defecation into our murine scRNAseq-derived NMF patterns using projectR. Transcriptomic data from intestinal tissues of patients with normal intestinal motility and those with obstructed defecation (OD) was procured from GEO (GSE101968). Non-negative matrix factorization (NMF), as implemented in the R package NNLM (github.com/linxihui/NNLM), was performed on the murine scRNAseq data using k=50 and default parameters; and cell weights for each pattern were grouped. Using projectR, the log 2 expression (log 2(rpkm+1)) from the human bulk RNA sequencing data from control and OD patients were projected into the murine scRNAseq-derived NMF patterns. The mean projection weights from Control and OD groups were tested for statistically significant differences using Students' t tests.

FIG. 19 shows a representation of selected and important dysregulated genes in patients with Obstructed Defecation. Supplemental data from Kim et al. was used to show that the important NENs-regulatory genes Gdnf and Ret, NENs-marker gene Snap25, and NENs-enriched gene Nos1 were all significantly downregulated in patients with obstructed defecation, when compared with control patient specimens. Similarly, MENs-specific genes Clic3, Cdh3, and Slc17a9 were all found to be significantly upregulated in patients with obstructed defecation, when compared with control patient specimens.

FIG. 20 is a graph demonstrating that maturing and aging ENS exhibit developmental plasticity. The neurons from the canonical NC lineage are progressively lost and are replaced by the neurons of the second lineage.

FIG. 21 is a graph demonstrating that reduced GDNF-RET signaling is associated with age-associated changes in the ENS.

FIG. 22 is a graph demonstrating that GDNF promotes maintenance of juvenile phenotype in the maturing ENS.

FIG. 23 is a graph demonstrating that GDNF is a juvenile protective factor (JPF) that reverses aging in the adult ENS.

FIG. 24 is a graph demonstrating that in the young adult murine small intestine (2 month old or P60 mice), the relative proportions or representation of the two lineages of neurons is sex-biased, with males having a higher representation of the mesodermal neurons, compared to females.

### DETAILED DESCRIPTION

Briefly, it was found that while the early post-natal ENS is derived from the canonical NC-lineage, this pattern changes rapidly as the ENS matures, due to the arrival and continual expansion of a novel population of Mesoderm-derived Enteric Neurons (MENs) which represent an equal proportion of the ENS in young adulthood and with increasing age, eventually outnumber the NC-derived Enteric Neurons (NENs). It was also found that, while the NEN population is regulated by glial derived neurotrophic factor (GDNF) signaling through its receptor RET, the MEN population is regulated by hepatocyte growth factor (HGF) signaling. Increasing HGF levels during maturation or by pharmacological dosing increase proportions of MENs. Similarly, decrease in GDNF with age decrease NENs; and increasing GDNF levels by pharmacological dosing increase NENs proportions in the adult ENS to impact intestinal motility.

These results indicate for the first time that the mesoderm is an important source of neurons in the second largest nervous system of the body. The increasing proportion of neurons of mesodermal lineage is a natural consequence of maturation and aging; further, this lineage can be expected to have vulnerabilities to disease that are distinct from those affecting the NEN population. These findings therefore provide a new paradigm for understanding the structure and function of the adult and aging ENS in health, age-related gut dysfunction and other acquired disorders of gastrointestinal motility.

Given the importance, size and complexity of the ENS, it contributes to the pathophysiology of gastrointestinal disorders and pathophysiological mechanisms underlying CNS disorders should also affect the ENS. Many neurotransmitters are common to the CNS and ENS and similar mechanisms govern development of both systems. The pathophysiology that gives rise to CNS disorders therefore may also be operative in the ENS. ENS deficits are accompany an increasing number of CNS disorders, from neurodevelopmental to neurodegenerative, and dysfunctional gastrointestinal manifestations might occur even before CNS symptoms become evident (Rao M, Gershon M D. The bowel and beyond: the enteric nervous system in neurological disorders. Nat Rev Gastroenterol Hepatol. 2016; 13(9):517-528. doi:10.1038/nrgastro.2016.107). Examples of disorders with both gastrointestinal and neurological consequences include transmissible spongiform encephalopathies, autistic spectrum disorders, Parkinson disease, Alzheimer disease, amyotrophic lateral sclerosis, and varicella zoster virus (VZV) infection.

### Compositions

Accordingly, in certain embodiments, a method of treating a subject with a gastrointestinal disorder related to age- and other disease associated structural alterations and neurodegeneration in the enteric nervous system comprises administering to the subject a pharmaceutical composition comprising a therapeutically effective amount of an agonist of RET receptor signaling and/or an antagonist of MET receptor signaling, thereby treating the subject. The therapeutically effective amount of the agonist of RET receptor signaling increases the number of neural crest (NC)-derived enteric neuron (NENs) cells relative to Mesodermal-lineage of enteric neuron (MENs) cells.

RET (REarranged during Transfection) is a receptor protein tyrosine kinase, which activates multiple signal transduction pathways. RET protein is composed of three domains: an extracellular ligand-binding domain, a transmembrane domain, and a cytoplasmic tyrosine kinase domain. The RET receptor tyrosine kinase (RTK) regulates key aspects of cellular proliferation and survival by regulating the activity of the mitogen-activated protein kinase (MAPK) and PI3K/Akt signaling pathways. RET also interacts directly with other kinases such as the epidermal growth factor receptor (EGFR) and hepatocyte growth factor receptor (MET) and the focal adhesion kinase (FAK). Furthermore, BRAF and p38MAPK are downstream targets of RET. Kinase inhibitors that simultaneously inhibit RET and its downstream targets.

Glial-cell line-derived neurotrophic Factor (GDNF) is a growth factor that regulates the health and function of neurons and other cells. GDNF binds to GDNF family receptor alpha 1 (GFRa1), and the resulting complex activates the RET receptor tyrosine kinase and subsequent downstream signals (Jmaeff, Sean et al. (2020). Small-molecule agonists of the RET receptor tyrosine kinase activate biased trophic signals that are influenced by the presence of GFRa1 co-receptors. Journal of Biological Chemistry. 295.jbc.RA119.011802. 10.1074/jbc.RA119.011802).

In certain embodiments, a pharmaceutical composition comprises a therapeutically effective amount of one or more agonists of RET signaling. In certain embodiments, an agonist of RET signaling comprises: glial derived neurotrophic factor (GDNF), GDNF mimics, GDNF analogs, juvenile protective factors (JPF), small molecules, peptide, oligonucleotides, antibodies, antibody fragments, single chain antibodies, antibody mimetics, peptoids, aptamers; enzymes, hormones, organic or inorganic molecules, natural or synthetic compounds.

Glial cell line-derived neurotrophic factor (GDNF) family ligands (GFLs) consist of GDNF, neurturin (NRTN), artemin (ARTN), and persephin (PSPNX Airaksinen M S, Saarma M. The GDNF family: signaling, biological functions and therapeutic value. Nat Rev Neurosci. 2002; 3(5):383-394. doi:10.1038/nrn812). All four GFLs-GDNF, ARTN, neurturin (NRTN), and persephin (PSPN)-signal through the transmembrane receptor tyrosine kinase RET. The binding specificity is provided by a cell surface-bound GPI-anchored GDNF family receptor α (GFRα): GDNF preferentially binds to GFRα1, NRTN to GFRα2, ARTN to GFRα3, and PSPN to GFRα4 (Airaksinen, M. S., and Saarma, M. (2002). The GDNF family: signalling, biological functions and therapeutic value. Nat. Rev. Neurosci. 3, 383-394. doi: 10.1038/nrn812; Sidorova. Y. A. et al., (2010). Persephin signaling through GFRalpha1: the potential for the treatment of Parkinson's disease. Mol. Cell. Neurosci. 44, 223-232. doi: 10.1016/j.mcn.2010.03.009). Ligand binding to GFRα/RET leads to autophosphorylation of RET kinase domains and subsequent activation of multiple intracellular signaling pathways including Akt, MAPK-Erk, Src, and JNK cascades (Airaksinen and Saarma, 2002). At least two alternative GDNF receptors are known: neural adhesion molecule (NCAM; Paratcha, G., Ledda, F., and Ibancz, C. F. (2003). The neural cell adhesion molecule NCAM is an alternative signaling receptor for GDNF family ligands. Cell 113, 867-879. doi: 10.1016/S0092-8674(03)00435-5) and heparan sulfate proteoglycan syndecan-3 (Bespalov, M. M., et al., (2011). Heparan sulfate proteoglycan syndecan-3 is a novel receptor for GDNF, neurturin, and artemin. J. Cell Biol. 192, 153-169. doi: 10.1083/jcb.201009136), which mediate some biological effects of GDNF.

In one embodiment, examples of polypeptides include members of the RET receptor ligand family of neurotrophic factors, such as glial cell line-derived neurotrophic factor (GDNF), Neurturin, Artemin, and Persephin. Other polypeptides include those that mediate downstream signaling by GDNF. In another embodiment, a compound is a GDNF mimic. Examples of GDNF mimics include small molecules and proteins, such as compounds that activate Dok-4 and/or Rap1GAP, compounds that block RhoA/ROCK signals, compounds that block PTEN signals, compounds that activate PI3K/Akt, compounds that activate cAMP, compounds that activate ERK1/2, and compounds that activate Rac1. Other agonists include BT3 (Yulia A. Sidorova et al., Front. Pharmacol., 21 Jun. 2017; doi.org/10.3389/fphar.2017.00365), naphthoquinone/quinolinedione family of small molecules (Sean Jmaeff et al., The Journal of Biological Chemistry. May 8, 2020, 295, 6532-6542).

Other examples of small molecule agonists include XIB4035 and BT13 (Larisa Ivanova, et al., ACS Omega 2018 3 (1), 1022-1030, DOI: 10.1021/acsomega.7b01932):

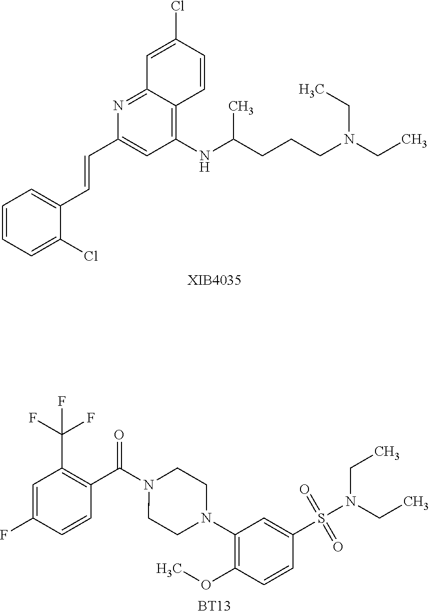

4-amino-8-hydroxynaphthalene-2,6-disulfonic acid (NSC37051) compound 7 (CAS 6271-90-5); 5-amino-4-hydroxynaphthalene-1,6-disulfonic acid (NSC37052) compound 8 (CAS 6271-89-2); (3Z)-6-amino-4-oxo-3-(phenylhydrazinylidene)naphthalene-2,7-disulfonic acid (NSC45189) compound 9 (CAS 6222-38-4); 5-amino-3-[[4-[4-[(4-amino-2-methylphenyl)diazenyl]phenyl]sulfanylphenyl]hydrazinylidene]-6-[(4-nitrophenyl)diazenyl]-4-oxonaphthalene-2,7-disulfonic acid (NSC65571) compound 15 (CAS 6950-40-9); 4-amino-3-[(2,5-dichlorophenyl)diazenyl]-5-oxo-6-[[4-[4-[2-(4-oxocyclohexa-2,5-dien-1-ylidene)hydrazinyl]phenyl]sulfanylphenyl]hydrazinylidene]naphthalene-2,7-disulfonic acid (NSC75661) compound 23; (3E)-5-amino-3-[[4-[4-[(4-amino-6-sulfonaphthalen-1-yl)diazenyl]phenyl]phenyl]hydrazinylidene]-6-[(4-nitrophenyl)diazenyl]-4-oxonaphthalene-2,7-disulfonic acid (NSC77520) compound 24; 4-amino-3-[(4-nitrophenyl)diazenyl]-5-oxo-6-[[4-[4-[2-(4-oxocyclohexa-2,5-dien-1-ylidene)hydrazinyl]phenyl]sulfanylphenyl]hydrazinylidene]naphthalene-2,7-disulfonic acid (NSC79723) compound 28; (3Z)-5-amino-3-[[4-[4-[(2,4-diamino-5-methylphenyl)diazenyl]phenyl]phenyl]hydrazinylidene]-6-[(2,5-dichlorophenyl)diazenyl]-4-oxonaphthalene-2,7-disulfonic acid (NSC79730) compound 29; 4-amino-3-[[4-[4-[(1-amino-5-sulfonaphthalen-2-yl)diazenyl]phenyl]phenyl]diazenyl]-5-oxo-6-(phenylhydrazinylidene)naphthalene-2,7-disulfonic acid (NSC79745) compound 35 (CAS 6486-54-0); (3Z)-5-amino-3-[[4-[4-[(2,4-diamino-3-methyl-6-sulfophenyl)diazenyl]phenyl]phenyl]hydrazinylidene]-6-[(3-nitrophenyl)diazenyl]-4-oxonaphthalene-2,7-disulfonic acid (NSC80903) compound 36 (PubChem Compound database (Kim, S., P. A. Thiessen, E. E. et al., (2016). “PubChem Substance and Compound databases.” Nucleic Acids Res 44(D1): D1202-1213)).

The c-mesenchymal-epithelial transition (c-MET) is a kinase receptor for hepatocyte growth factor (HGF), is well-known for its roles in driving tumorigenesis (Granito, A. et al., c-MET receptor tyrosine kinase as a molecular target in advanced hepatocellular carcinoma. J. Hepatocell. Carcinoma 2015, 2, 29-38; Boromand, N. et al., Clinical and prognostic value of the c-Met/HGF signaling pathway in cervical cancer. J. Cell. Physiol. 2017, 233, 4490-4496. Konstorum, A., Lowengrub, J. S. Activation of the HGF/c-Met axis in the tumor microenvironment: A multispecies model. J. Theor. Boil. 2017, 439, 86-99). It is a disulfide-linked heterodimer consisting of a highly glycosylated extracellular α-subunit and a transmembrane p-subunit. Upon binding to the HGF, c-MET triggers dimerization of two subunits, leading to autophosphorylation of tyrosine residues in cytoplasmic domain (Bao, Q. L. et al., The role of HGF/c-MET signaling pathway in lymphoma. J. Hematol. Oncol. 2016, 9, 135. Hu, C. T. et al., The therapeutic targeting of HGF/c-Met signaling in hepatocellular carcinoma: Alternative approaches. Cancers 2017, 9, 58). Then, phosphorylation of these tyrosine residues (Tyr1349 and Tyr1356) results in an activated C-terminal docking site, which has been identified to be able to recruit intracellular adaptor proteins (Furge, K. A. et al., Met receptor tyrosine kinase: Enhanced signaling through adapter proteins. Oncogene 2000, 19, 5582-5589). These events trigger several downstream signaling pathways such as phosphoinositide 3-kinase/threonine-protein kinase (PI3K/AKT) pathway, wingless-related integration site (Wnt) pathway, and others Arnold, L. et al., Activated HGF-c-Met axis in head and neck cancer. Cancers 2017, 9, 169. Stanley, A. et al., Synergistic effects of various Her inhibitors in combination with IGF-1R, C-MET and Src targeting agents in breast cancer cell lines. Sci. Rep. 2017, 7, 3964). Moreover, HGF/c-MET induced cell proliferation, migration, survival, invasion, differentiation, and epithelial-mesenchymal transition (EMT), promoting the progression of tumorigenesis (Hongli Zhang et al., “HGF/c-MET: A Promising Therapeutic Target in the Digestive System Cancers”, Int. J. Mol. Sci. 2018, 19, 3295; doi:10.3390/ijms19113295; 4,11,12).

Accordingly in certain embodiments, a pharmaceutical composition comprises a therapeutically effective amount of one or more antagonists of MET receptor signaling. Examples of inhibitors of MET receptor include: Tepotinib MSC2156119J (a highly selective ATP-competitive c-MET inhibitor), Tivantinib ARQ197 (a non-ATP competitive selective small-molecular inhibitor), SU11274, cabozantinib capmatinib, golvatinib, foretinib, SARI25844, other selective small molecule c-Met inhibitors such as KRC-408, KRC-00715, and Simm530, and multi-targeted kinase inhibitor T-1840383. An example of a small an HGF inhibitor is SRI 31215. Other antagonists include anti-HGF and anti-c-MET monoclonal antibodies including those directed against the extracellular combination of c-MET and HGF. Rilotumumab (AMG 102) is a humanized IgG2 monoclonal antibody that selectively binds to HGF. Onartuzumab is a humanized monoclonal antibody that binds to the c-MET extracellular domain. Anti-c-Met monoclonal antibodies ABT-700 and LY2875358. Other examples include miRNAs. (Zhang. Hongli et al. “HGF/c-MET: A Promising Therapeutic Target in the Digestive System Cancers.” International journal of molecular sciences vol. 19,11 3295. 23 Oct. 2018. doi:10.3390/ijms19113295).

In certain embodiments a pharmaceutical composition comprises one or more RET inhibitors. Examples include Alectinib, Apatinib, BLU-667, Lenvatinib, Ponatinib, Sunitinib, Sitravatinib, BLU-667, LOXO-292, Cabozantinb, Vandetanib, Atrial Natriuretic Peptide (1-28) (human, porcine) (SLRRSSCFGGRMDRIGAQSGLGCNSFRY SEQ ID NO: 1), Atrial Natriuretic Peptide (1-28) (rat) (SLRRSSCFGGRIDRIGAQSGLGCNSFRY; SEQ ID NO: 2),

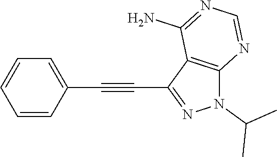 [SMILES: CC(C)n1nc(C#Cc2ccccc2)c2c(N)ncnc21]

1-(1-Methylethyl)-3-(2-phenylethynyl)-1H-pyrazolo[3,4-d]pyrimidin-4-amine,

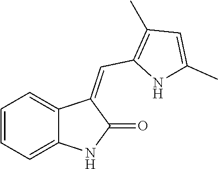 [SMILES: Cc1cc(C)c(/C=C2\C(=O)Nc3ccccc32)[nH]1]

3-[(3,5-Dimethyl-1H-pyrrol-2-yl)methylene]-1,3-dihydro-2H-indol-2-one,

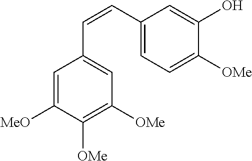 [SMILES: COc1ccc(/C=C\c2cc(OC)c(OC)c(OC)c2)cc1O]

2-Methoxy-5-[(1Z)-2-(3,4,5-trimethoxyphenyl)ethenyl]phenol.

In certain embodiments, a pharmaceutical composition comprises one or more MET receptor signaling agonists. Examples include, aML5-PEG3, aML5-PEG11, aML5-C6.

### Screening Assays

In certain aspects, methods for identifying agonists of RET signaling and/or one or more antagonists of MET receptor signaling for use in the treatment of gastrointestinal disorders related to age- and other disease associated structural alterations and neurodegeneration in the enteric nervous system. Accordingly, in certain embodiments, the disclosure features methods of identifying compounds useful in modulating populations of NEN and MEN cells, the methods featuring screening or assaying for compounds that modulate, e.g., activate or increase, or inhibit or decrease, RET signaling and/or MET receptor signaling, or biologically active portions thereof. In exemplary aspects, the methods comprise: providing an indicator composition, e.g. a cell expressing a RET or MET receptor, or biologically active portions thereof; contacting the indicator composition with each member of a library of test compounds; and selecting from the library of test compounds a compound of interest that modulates, for example, interaction and/or RET or MET receptor signaling, or biologically active portions thereof, wherein the ability of a compound to modulate signaling is indicated by, for example, FRET, BRET, luciferase assays, phosphorylation assays, receptor-ligand binding assays, proliferation or lack thereof, of NEN or MEN cells, Western blots, immunoassays, hybridization, etc., as compared to the control, e.g. in the absence of the compound (Sidorova, Y. A., Matlik, K., Paveliev, M., Lindahl, M., Piranen, E., Milbrandt, J., et al. (2010). Persephin signaling through GFRalpha1: the potential for the treatment of Parkinson's disease. Mol. Cell. Neurosci. 44, 223-232. doi: 10.1016/j.mcn.2010.03.009).

As used herein, the term “contacting” (i.e., contacting a cell e.g. a cell, with a compound) includes incubating the compound and the cell together in vitro (e.g., adding the compound to cells in culture) as well as administering the compound to a subject such that the compound and cells of the subject are contacted in vivo. The term “contacting” does not include exposure of cells to a RET receptor signaling and/or MET receptor signaling modulator that may occur naturally in a subject (i.e., exposure that may occur as a result of a natural physiological process).

As used herein, the term “test compound” or “candidate therapeutic agent” refers to a compound that has not previously been identified as, or recognized to be, a modulator of the activity being tested. The term “library of test compounds” refers to a panel comprising a multiplicity of test compounds.

As used herein, the term “indicator composition” refers to a composition that includes a protein of interest (e.g., RET receptor, MET receptor or a molecule in a biological pathway involving these receptors, e.g., GDNF, HGF), for example, a cell that naturally expresses the protein, a cell that has been engineered to express the protein by introducing one or more of expression vectors encoding the protein(s) into the cell, or a cell free composition that contains the protein(s) (e.g., purified naturally-occurring protein or recombinantly-engineered protein(s)).

As used herein, the term “cell” includes prokaryotic and eukaryotic cells. In one embodiment, a cell is a bacterial cell. In another embodiment, a cell is a fungal cell, such as a yeast cell. In another embodiment, a cell is a vertebrate cell, e.g., an avian or mammalian cell. In certain embodiments, a cell is a murine or human cell. As used herein, the term “engineered” (as in an engineered cell) refers to a cell into which a nucleic acid molecule e.g., encoding RET or MET protein (e.g., a spliced and/or unspliced form of RET, MET) has been introduced.

As used herein, the term “cell free composition” refers to an isolated composition, which does not contain intact cells. Examples of cell free compositions include cell extracts and compositions containing isolated proteins.

Screening of test compounds suitably include, animal models, cell-based systems and non-cell based systems. Preferably, identified genes, variants, fragments, or oligopeptides thereof are used for identifying agents of therapeutic interest, e.g. by screening libraries of compounds or otherwise identifying compounds of interest by any of a variety of drug screening or analysis techniques. The gene, allele, fragment, or oligopeptide thereof employed in such screening may be free in solution, affixed to a solid support, borne on a cell surface, or located intracellularly.

The methods of screening using screening assays to identify, from a library of diverse molecules, one or more compounds having a desired activity e.g. RET, MET receptor signaling activity. A “screening assay” is a selective assay designed to identify, isolate, and/or determine the structure of, compounds within a collection that have a preselected activity. By “identifying” it is meant that a compound having a desirable activity is isolated, its chemical structure is determined (including without limitation determining the nucleotide and amino acid sequences of nucleic acids and polypeptides, respectively) the structure of and, additionally or alternatively, purifying compounds having the screened activity). Biochemical and biological assays are designed to test for activity in a broad range of systems ranging from protein-protein interactions, enzyme catalysis, small molecule-protein binding, to cellular functions. Such assays include automated, semi-automated assays and HTS (high throughput screening) assays.

In HTS methods, many discrete compounds are preferably tested in parallel by robotic, automatic or semi-automatic methods so that large numbers of test compounds are screened for a desired activity simultaneously or nearly simultaneously. It is possible to assay and screen up to about 6,000 to 20,000, and even up to about 100,000 to 1,000,000 different compounds a day using the integrated systems of the invention.

Typically in HTS, target molecules are administered or cultured with isolated cells with modulated receptors, including the appropriate controls.

In certain embodiments, a screening assay is used to identify RET agonists or antagonists or MET agonists or MET antagonists. Screening assays are known in the art, for example, Watson, Amanda J et al. “Identification of selective inhibitors of RET and comparison with current clinical candidates through development and validation of a robust screening cascade.” F1000Research vol. 5 1005. 26 May 2016, doi:10.12688/f1000research.8724.2; Mendoza L. Clinical development of RET inhibitors in RET-rearranged non-small cell lung cancer: Update. Oncol Rev. 2018; 12(2):352. Published 2018 Jul. 10. doi:10.4081/oncol.2018.352, Miao W, Sakai K, imamura R, et al. MET Activation by a Macrocyclic Peptide Agonist that Couples to Biological Responses Differently from HGF in a Context-Dependent Manner. Int J Mol Sci. 2018; 19(10):3141. Published 2018 Oct. 12. doi:10.3390/ijms19103141 which are incorporated by reference in their entirety. MET agonist screening kits are also available commercially, for example ADP-GLO™ Kinase Assay (Promega).

The ability of the test compound to modulate GDNF binding to RET or HGF to MET can also be determined. Determining the ability of the test compound to modulate GDNF or HGF binding to RET or MET respectively, can be accomplished, for example, completive ELISA. The coupling of, for example, GDNF with a radioisotope or enzymatic label such that binding of GDNF to RET can be determined by detecting the labeled GDNF in a complex. Alternatively, the test compound or candidate agent could be coupled with a radioisotope or enzymatic label to monitor the ability of the test compound to bind to the RET or MET receptor. See, also the examples section which follows for a detailed description of assays performed. Determining the ability of the test compound to bind to RET or MET can be accomplished, for example, by coupling the compound with a radioisotope or enzymatic label such that binding of the compound to RET or MET can be determined by detecting the labeled compound in a complex. For example, targets can be labeled with ¹²⁵I, ³⁵S, ¹⁴C, or ³H, either directly or indirectly, and the radioisotope detected by direct counting of radio emission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

It is also within the scope of this disclosure to determine the ability of a compound to interact with RET or MET without the labeling of any of the interactants. For example, a microphysiometer can be used to detect the interaction of a compound with RET or MET without the labeling of either the compound or the RET or MET (McConnell, H. M., et al. 1992. Science 257, 1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and RET or MET.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell. In certain embodiments, the cell is a human cell. The cells can express endogenous RET or MET or can be engineered to do so. For example, a cell that has been engineered to express the RET or MET receptors can be produced by introducing into the cell an expression vector encoding the protein.

In another embodiment, the indicator composition is a cell free composition. the RET or MET receptors expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment of the above assay methods, it may be desirable to immobilize either the receptor or test compound, for example, to facilitate separation of complexed from uncomplexed forms of one or both of the receptor and test compound, or to accommodate automation of the assay.

Binding of a test compound to a receptor in the presence and absence of a test compound, can be accomplished in any vessel suitable for containing the reactants. Examples of such vessels include microtiter plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or receptor protein, and the mixture incubated under conditions conducive to complex formation (e.g., at physiological conditions for salt and pH). Following incubation, the beads or microtiter plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques. Methods for detecting such complexes, in addition to those described above for the GST-immobilized complexes, include immunodetection of complexes, as well as enzyme-linked assays which rely on detecting an enzymatic activity.

Test compounds include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Test compounds may comprise functional chemical groups that interact with proteins and/or DNA.

Test compounds may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Another technique for drug screening provides for high throughput screening of compounds having suitable binding affinity to the protein of interest (see, e.g., Geysen et al., 1984, PCT application WO84/03564). In this method, large numbers of different small test compounds are synthesized on a solid substrate. The test compounds are reacted with identified genes, or fragments thereof, and washed. Bound molecules are then detected by methods well known in the art. Alternatively, non-neutralizing antibodies can be used to capture the peptide and immobilize it on a solid support.

In one embodiment, screening comprises contacting each cell culture with a diverse library of member compounds, some of which are ligands of the target, under conditions where complexes between the target and ligands can form, and identifying which members of the libraries are present in such complexes. In another non limiting modality, screening comprises contacting a target enzyme with a diverse library of member compounds, some of which are inhibitors (or activators) of the target, under conditions where a product or a reactant of the reaction catalyzed by the enzyme produce a detectable signal. In the latter modality, inhibitors of target enzyme decrease the signal from a detectable product or increase a signal from a detectable reactant (or vice-versa for activators).

In the high throughput assays, either soluble or solid state, it is possible to screen up to several thousand different modulators or ligands in a single day. This methodology can be used for proteins in vitro, or for cell-based or membrane-based assays. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non-covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder. A number of tags and tag binders can be used, based upon known molecular interactions well described in the literature. Similarly, any haptenic or antigenic compound can be used in combination with an appropriate antibody to form a tag/tag binder pair. Thousands of specific antibodies are commercially available and many additional antibodies are described in the literature Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with .beta.-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

High throughput screening can be used to measure the effects of drugs on complex molecular events, e.g. regulation of phosphorylation and/or acetylation. Multicolor fluorescence permits multiple targets and cell processes to be assayed in a single screen. Cross-correlation of cellular responses will yield a wealth of information required for target validation and lead optimization.

In another aspect, a method for analyzing cells comprising providing an array of locations which contain multiple cells wherein the cells contain one or more fluorescent reporter molecules; scanning multiple cells in each of the locations containing cells to obtain fluorescent signals from the fluorescent reporter molecule in the cells; converting the fluorescent signals into digital data; and utilizing the digital data to determine the distribution, environment or activity of the fluorescent reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles. Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J. Biol. Chem. 272:27497-27500; Southwick et al., (1990), Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue. The luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to a protein of interest as previously described (Chalfie and Prasher U.S. Pat. No. 5,491,084; Cubitt et al. (1995), Trends in Biochemical Science 20:448-455).

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371; Thomas et al. (1979), Biochemistry 18:2210-2218). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner—for example, with an auto-sampler.

The particular label or detectable moiety or tag used in the assay is not a critical aspect of the invention. The detectable group can be any material having a detectable physical or chemical property. Such detectable labels have been well developed in the field of immunoassays and, in general, most labels useful in such methods can be applied to the present invention. Thus, a label is any composition detectable by spectroscopic, photochemical, biochemical, immunochemical, electrical, optical, or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound.

The molecules can also be conjugated directly to signal generating compounds, e.g., by conjugation with an enzyme or fluorophore. Any type of enzyme label can be used as long as they do not interfere with one of the desired outputs of the assay, e.g. expression and/or acylation/deacetylation state etc. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge-coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

The method further provides a substance, e.g. a ligand, identified or identifiable by identification or screening methods or use of the invention. Such substances may be capable of stimulating, promoting, activating or inhibiting, directly or indirectly, the activity of a ligand-receptor. The term substances includes substances that do not directly bind the receptor and induce expression or inhibition of expression of the receptor or promote, activate or inhibit a function, but instead indirectly induce expression of the peptide biomarker or promote/activate a function of the peptide biomarker, or phosphorylate, acetylate etc. Ligands are also included in the term substances; ligands of the invention (e.g. a natural or synthetic chemical compound, peptide, aptamer, oligonucleotide, antibody or antibody fragment) are capable of binding, preferably specific binding, to a peptide biomarker.

### Formulations for Administration

In certain embodiments, the pharmaceutical compositions are administered orally. Oral administration in context of the present disclosure means the introduction of the composition via the mouth. In certain embodiments, the composition is a solid dosage form, such as in the form of a pellet, granule, micro particle, nano particle, mini tablet, capsule or tablet coated with a coating material that prevents the release of the composition before the ileocolonic region of the intestine. The ileocolonic region is the region of the gastrointestinal tract where the small intestine merges with the large intestine, i.e. the terminal ileum.

The compositions can be encapsulated, e.g. nanoparticles, in a tablet form or encapsulated particles in a suspension. The compositions can be coated so that the pharmaceutical agents are released in the intestines and not in the stomach. Coating materials for the targeted release of a composition in the large intestinal lumen are known in the art. They can be subdivided into coating materials that disintegrate above a specific pH, coating materials that disintegrate after a specific residence time in the gastrointestinal tract and coating materials that disintegrate due enzymatic triggers specific to the microflora of the large intestine. Coating materials of these three different 15 categories for targeting to the large intestine have been reviewed for example in Bansal et al. (Polim. Med. 2014, 44, 2, 109-118). Coating materials include, for example, poly vinyl acetate phthalate, cellulose acetate trimellitate, hydroxypropyl methylcellulose phthalate HP-50, hydroxypropyl methylcellulose phthalate HP-55, hydroxypropyl methylcellulose phthalate HP-55S, hydroxypropyl methylcellulose acetate succinate, cellulose acetate phthalate, acrylic acid copolymer, Eudragit L100-55, Eudragit L30D-55, Eudragit L-100, Eudragit L12.5, Eudragit S-100, Eudragit S12.5, Eudragit FS30D, hydroxyl propylethyl cellolose phthalate, PEG 6000, Ac-di-sol, Talc, hydroxy propyl methyl cellulose acetate succinate (HPMCAS), hydroxy ethyl cellulose, ethylcellulose, microcrystalline cellulose, hydroxy propyl methyl cellulose, chondroitin sulphate, pectin, guar gum, chitosan, lactose, maltose, cellobiose, inulin, cyclodextrin, lactulose, raffinose, stachyose, alginate, dextran, xantham gum, guar gum, starch, tragacanth, locust bean gum, cellulose, arabinogalactan, amylose and combinations thereof.

In some instances, the formulation is distributed or packaged in a liquid form (e.g., suspension) for oral administration, for administration as an enema, or administration by instillation into a body cavity or lumen. Alternatively, formulations for non-injectable administration can be packaged as a solid, obtained, for example, by lyophilization of a suitable liquid formulation. The solid can be reconstituted with an appropriate carrier or diluent prior to administration.

Solutions and suspensions of the nanoparticles and/or microparticles can be prepared in water or another solvent or dispersing medium suitably mixed with one or more pharmaceutically acceptable excipients including, but not limited to, surfactants, dispersants, emulsifiers, pH modifying agents, and combination thereof.

The solution formulation is typically buffered to a pH of 3-8 for administration upon reconstitution. Suitable buffers are well known by those skilled in the art and some examples of useful buffers are acetate, borate, carbonate, citrate, and phosphate buffers.

Solutions, suspensions, or emulsions for administration may also contain one or more tonicity agents to adjust the isotonic range of the formulation. Suitable tonicity agents are well known in the art. Examples include glycerin, mannitol, sorbitol, sodium chloride, and other electrolytes.

Solutions, suspensions, or emulsions for administration may also contain one or more surfactants. Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or nanoparticles and/or microparticles.

Gelatin Capsules and Tablets: Tablets and inserts/suppositories can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations are prepared using pharmaceutically acceptable carriers including but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Optional pharmaceutically acceptable excipients include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar. The usual diluents include inert powdered substances such as starches, powdered cellulose, especially crystalline and microcrystalline cellulose, sugars such as fructose, mannitol and sucrose, grain flours, and similar edible powders. Typical diluents include, for example, various types of starch, lactose, mannitol, kaolin, calcium phosphate or sulfate, inorganic salts such as sodium chloride, and powdered sugar. Powdered cellulose derivatives are also useful.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin, and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

A lubricant can be used in a tablet formulation to prevent the tablet and punches from sticking in the die to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (POLYPLASDONE™ XL from GAF Chemical Corp.).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; vitamin E, tocopherol and its salts; sulfites such as sodium metabisulphite; cysteine and its derivatives; citric acid; propyl gallate, and butylated hydroxyanisole (BHA).

In some embodiments, the weight percent of the gel particles in the tablet or capsule formulations (with excipients) is between about 2% and about 80%, or between about 5% and about 70%, or between about 10% and about 60%. In some embodiments, the excipients include sodium starch glycolate (as a disintegrant) and mannitol (as a filler). In some embodiments, the weight percent of the gel particles in the tablet or capsule formulations (with excipients) is between about 2% and about 80%, or between about 5% and about 70%, or between about 10% and about 60%. In some embodiments, the excipients include sodium starch glycolate (as a disintegrant) and mannitol (as a filler).

Rectal Inserts or Suppositories: In certain embodiments, the formulation is administered rectally as a suppository, insert or enema. Rectal inserts or suppositories are typically formed by the same techniques as tablets, with additional excipient for comfort once inserted, such as increased amounts of inserts. The size and shape are selected based on the route of administration. These shapes, sizes, and excipients are well known to those in the pharmaceutical compounding art.

Enteric, Delayed or Pulsatile Release Formulations and Blended Formulations: In certain embodiments, the formulations are administered into the colon, e.g. by instillation or other methods used in the arts. A number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

Coatings can be applied to the particles, tablets, capsules, or inserts to modify release and to increase residence time at the site of delivery. The coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine from the clinical studies.

Coatings may be formed with a different ratio of water soluble polymer, water insoluble polymers and/or pH dependent polymers, with or without water insoluble/water soluble non-polymeric excipient, to produce the desired release profile. The coating is either performed on dosage form (matrix or simple) which includes, but are not limited to, tablets (compressed with or without coated beads), capsules (with or without coated beads), beads, particle compositions, and “ingredient as is” formulated as, but not limited to, suspension form or as a sprinkle dosage form.

Additionally, the coating material may contain conventional carriers such as plasticizers, pigments, colorants, glidants, stabilization agents, pore formers and surfactants.

The peptides embodied here, can be lyophilized, loaded onto microfibers which can be adsorbed onto microcrystalline cellulose beads (e.g., 60-250 μm mesh, or as large as 1,000 μm mesh) using a dry layering or suspension layering process. The microbeads are then coated by a fluidized bed coating process. Examples of coatings include pH responsive enteric coating, sustained released coating, and controlled release coating. In some embodiments, multi-layered coatings can be applied. The coated microbeads can be administered as a solid oral dosage form by loading them into a capsule or table. Alternatively, the coated microbeads can be suspended in water, buffer or other media and delivered as a liquid dosage form. Other buffering agents and excipients may be added to the liquid dosage form.

Enteric Coatings: The particles, tablets, capsules, or inserts may be coated to delay release to after the particles have passed through the acidic environment of the stomach. These materials are usually referred to as enteric coatings. For example, enteric polymers become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

Exemplary enteric polymers include polymethacrylates and derivatives thereof, such as ethyl methacrylate-methacrylic acid copolymer and those sold under the tradename EUDRAGIT™, naturally occurring cellulosic polymers (e.g., cellulose acetate succinate, cellulose acetate phthalate, hydroxy propyl methyl cellulose phthalate, and hydroxy propyl methyl cellulose acetate succinate) and other polysaccharides (e.g., sodium alignate, pectin, chitosan) or semi-synthetic or synthetic derivatives thereof, poly(2-vinylpyridine-co-styrene), polyvinyl acetate phthalate, shellac, fatty acids (e.g., stearic acid), waxes, plastics, and plant fibers.

Exemplary gastric resistant natural polymers include, but are not limited to, pectin and pectin-like polymers which typically consist mainly of galacturonic acid and galacturonic acid methyl ester units forming linear polysaccharide chains. Typically these polysaccharides are rich in galacturonic acid, rhamnose, arabinose and galactose, for example the polygalacturonans, rhamnogalacturonans and some arabinans, galactans and arabinogalactans. These are normally classified according to the degree of esterification. In high (methyl) ester (“HM”) pectin, a relatively high portion of the carboxyl groups occur as methyl esters, and the remaining carboxylic acid groups are in the form of the free acid or as its ammonium, potassium, calcium or sodium salt. Useful properties may vary with the degree of esterification and with the degree of polymerization.

Pectin, in which less than 50% of the carboxyl acid units occur as the methyl ester, is normally referred to as low (methyl) ester or LM-pectin. In general, low ester pectin is obtained from high ester pectin by treatment at mild acidic or alkaline conditions. Amidated pectin is obtained from high ester pectin when ammonia is used in the alkaline deesterification process. In this type of pectin some of the remaining carboxylic acid groups have been transformed into the acid amide. The useful properties of amidated pectin may vary with the proportion of ester and amide units and with the degree of polymerization.

Synthetic enteric polymers include, but are not limited to, acrylic acid polymers and copolymers, preferably formed from acrylic acid, methacrylic acid, methyl acrylate, ethyl acrylate, methyl methacrylate and/or ethyl methacrylate, and methacrylic resins that are commercially available under the tradename EUDRAGIT™ (Rohm Pharma; Westerstadt, Germany), including EUDRAGIT™ L30 D-55 and L100-55 (soluble at pH 5.5 and above), EUDRAGIT™ L-100 (soluble at pH 6.0 and above), EUDRAGIT™ S (soluble at pH 7.0 and above, as a result of a higher degree of esterification), and EUDRAGIT™ NE, RL and RS (water-insoluble polymers having different degrees of permeability and expandability).

The enteric coating is generally present in an amount less than about 10% by weight of the composition (e.g., gel particles, tablets, or capsules), preferably from about 2 to about 8% by weight of the composition.

The dosage units may be coated with the delayed release polymer coating using conventional techniques, e.g., using a conventional coating pan, an airless spray technique, or fluidized bed coating equipment (with or without a Wurster insert). See Pharmaceutical Dosage Forms: Tablets, Eds. Lieberman et al. (New York: Marcel Dekker, Inc., 1989), and Ansel et al., Pharmaceutical Dosage Forms and Drug Delivery Systems, 6th Ed. (Media, Pa.: Williams & Wilkins, 1995) for detailed information concerning materials, equipment and processes for preparing tablets and delayed release dosage forms.

Extended Release Drug/Particle Blends: One method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

The extended release formulations are generally prepared as diffusion or osmotic systems, for example, as described in “Remington—The Science and Practice of Pharmacy” (20th Ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, CARBOPOL™ 934, and polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion. The formulations with different drug release mechanisms can be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours, and similar edible powders. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders include substances such as starch, gelatin, and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose, and waxes can also serve as binders. A lubricant can be used in a tablet formulation to prevent the tablet and punches from sticking in the die. The lubricant is chosen from such slippery solids as talc, magnesium and calcium stearate, stearic acid, and hydrogenated vegetable oils.

Extended release tablets or inserts containing wax materials are generally prepared using methods known in the art such as a direct blend method, a congealing method, and an aqueous dispersion method. In a congealing method, the drug is mixed with a wax material and either spray-congealed or congealed and screened and processed.

Delayed Release Dosage Forms: Delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate, and carboxymethylcellulose sodium; acrylic acid polymers and copolymers, formed from acrylic acid, methacrylic acid, methyl acrylate, ethyl acrylate, methyl methacrylate and/or ethyl methacrylate, and other methacrylic resins that are commercially available under the tradename EUDRAGIT™ (Rohm Pharma; Westerstadt, Germany), including EUDRAGIT™ L30D-55 and L100-55 (soluble at pH 5.5 and above), EUDRAGIT™ L-100 (soluble at pH 6.0 and above), EUDRAGIT™ S (soluble at pH 7.0 and above, as a result of a higher degree of esterification), and EUDRAGIT™ NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The coating weights for particular coating materials may be readily determined by those skilled in the art by evaluating individual release profiles for tablets, beads and granules prepared with different quantities of various coating materials. It is the combination of materials, method and form of application that produce the desired release characteristics.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Pulsatile Release Formulations: By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design, e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In certain embodiments, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than three tablets, dosage forms housing more than three tablets can be utilized.

Alternatively, each dosage unit in the capsule may comprise a plurality of drug-containing beads, granules or particles. Drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. Drug-containing “granules” and “particles” comprise drug particles that may or may not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

In another embodiment, the individual dosage units are compacted in a single tablet, and may represent integral but discrete segments thereof (e.g., layers), or may be present as a simple admixture. For example, drug-containing beads, granules or particles with different drug release profiles (e.g., immediate and delayed release profiles) can be compressed together into a single tablet using conventional tableting means.

The compositions described herein are suitable for use in a variety of drug delivery systems. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The effective dose can vary, depending upon factors such as the condition of the patient, the severity of the viral infection, and the manner in which the pharmaceutical composition is administered. The effective dose of compounds will of course differ from patient to patient, but in general includes amounts starting where desired therapeutic effects occur but below the amount where significant side effects are observed.

The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

Film-Forming Polymers for Coating Capsules: The film-forming composition can be used to prepare soft or hard shell gelatin capsules which can encapsulate a liquid or semi-solid fill material or a solid tablet (e.g., SOFTLET™) containing an active agent and one or more pharmaceutically acceptable excipients. Alternatively, the composition can be administered as a liquid with an active agent dissolved or dispersed in the composition. Exemplary film-forming natural polymers include, but are not limited to, gelatin and gelatin-like polymers. In certain embodiments, the film-forming natural polymer is gelatin. A number of other gelatin-like polymers are available commercially. The film-forming natural polymer is present in an amount from about 20 to about 40% by weight of the composition, or from about 25 to about 40% by weight of the composition.

The film-forming composition can be used to prepare soft or hard capsules using techniques well known in the art. For example, soft capsules are typically produced using a rotary die encapsulation process. Fill formulations are fed into the encapsulation machine by gravity. The capsule shell can contain one or more plasticizers selected from the group consisting of glycerin, sorbitol, sorbitans, maltitol, glycerol, polyethylene glycol, polyalcohols with 3 to 6 carbon atoms, citric acid, citric acid esters, triethyl citrate and combinations thereof. In addition to the plasticizer(s), the capsule shell can include other suitable shell additives such as opacifiers, colorants, humectants, preservatives, flavorings, and buffering salts and acids.

Opacifiers are used to opacify the capsule shell when the encapsulated active agents are light sensitive. Suitable opacifiers include titanium dioxide, zinc oxide, calcium carbonate, and combinations thereof. Colorants can be used for marketing and product identification/differentiation purposes. Suitable colorants include synthetic and natural dyes and combinations thereof.

Humectants can be used to suppress the water activity of the soft gel. Suitable humectants include glycerin and sorbitol, which are often components of the plasticizer composition. Due to the low water activity of dried, properly stored soft gels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

### EXAMPLES

### Example 1: Age-Specific Mechanisms Regulate Neural Crest and Mesodermal Contribution to the Enteric Nervous System in Health and Disease

The enteric nervous system (ENS) is a large collection of neurons and related cells that resides within the gastrointestinal wall and regulates gut motility and secretion along with modulating epithelial and immune cell function^{1,2}. During fetal development, the mammalian ENS is populated by neurons and glia derived from neural crest (NC)-derived precursors³⁻⁹. These precursors follow diverse migratory routes to colonize and innervate various parts of the gut before birth¹⁰⁻¹². It is not clear, however, that this lineage persists in its entirety in the adult gut, as indicated by the observed lack of expression of fluorescent reporter protein in a subpopulation of adult enteric neurons in NC-lineage-traced mice^{13,14}. Alternative sources of enteric neurons that have been proposed in the literature include the ventral neural tube (VENT)¹⁵, or the Pdx1-expressing pancreatic endoderm¹⁴, but the interpretation of these studies has been limited by the lack of robust lineage markers for non-NC derived neurons¹⁶. In addition, while prior studies have documented cellular changes to the ageing ENS¹⁷, the developmental mechanisms behind these changes are unknown. Thus, confirmation of a second, distinct lineage of enteric neurons in adults is important for our understanding of the healthy post-natal development and aging of the ENS, as well as for the pathogenesis of acquired disorders of the ENS.

The results obtained herein provide evidence for the first time that the mesoderm and not the neuroectoderm is an important source of neurons in the second largest nervous system of the body. The increasing proportion of neurons of mesodermal lineage is a natural consequence of maturation and aging; further, this lineage can be expected to have vulnerabilities to disease that are distinct from those affecting the NEN population. These findings therefore provide a new paradigm for understanding the structure and function of the adult and aging ENS in health, age-related gut dysfunction and other acquired disorders of gastrointestinal motility.

### Materials and Methods

Animals:

Experimental protocols were approved by The Johns Hopkins University's Animal Care and Use Committee in accordance with the guidelines provided by the National Institutes of Health. Presence of vaginal plug was ascertained as 0.5 days post-fertilization and this metric was used to calculate age of mice. Only male mice were used for the studies detailed in this report. The Wnt1-cre:Rosa26-tdTomato lineage-traced line was generated as detailed before by breeding the B6.Cg-Tg(Wnt1-cre) with the Ail4 transgenic mouse line (Jax #: 007914) containing the Rosa26-tdTomato transgene^{71,78,79}. Pax3-cre:Rosa26-tdTomato lineage-traced line was generated by breeding the Ai9 transgenic mouse line (Jax #: 007909) with the Pax3-cre transgenic mouse (Jax #: 005549). The Wnt1-cre:Hprt-tdTomato mouse was generated by breeding our aforementioned Wnt1-cre transgenic mouse line with the Hprt-tdTomato transgenic mouse line (Jax #: 021428, kind gift from Prof. Jeremy Nathans). Mesp1-cre:Rosa26-tdTomato mice were generated by breeding the Mesp1-cre transgenic mice⁸⁰ with the Ail4 transgenic mice. Ret^{+/CFP} mice (MGI:3777556) were inter-bred to get a colony of adult Ret^{+/+} and Ret^{+/CFP} mice. Ret^{CFP/CFP} mice died at or before term. Tek-cre:Hprt-tdTomato mice were generated by breeding Tek-cre transgenic mice (also known as Tie2-cre; Jax #: 004128) with the Hprt-tdTomato transgenic mouse line. 17-month-old male C57BL/6 mice from the aging mouse colony of the National Institute of Aging were procured for the GDNF-treatment experiment.

Human Tissues:

Human tissues were obtained under IRB protocol IRB00181108 that was approved by Institutional Review Board at the Johns Hopkins University. Pathological normal specimens of human duodenum and colon were obtained post-resection. Tissues were obtained from adult donors and were de-identified such that the exact age, gender, and ethnicity of the donors was unknown.

Tissue Preparation:

Mice were anesthetized with isoflurane and sacrificed by cervical dislocation. A laparotomy was performed, and the ileum was removed and lavaged with PBS containing penicillin-streptomycin (PS; Invitrogen), then cut into 1-cm-long segments and placed over a sterile plastic rod. A superficial longitudinal incision was made along the serosal surface and the LM-MP was peeled off from the underlying tissue using a wet sterile cotton swab 71 and placed in Opti-MEM medium (Invitrogen) containing Pen-Strep (Invitrogen). The tissue was then laid flat and fixed with freshly prepared ice cold 4% paraformaldehyde (PFA) solution for 45 minutes in the dark to preserve fluorescence intensity and prevent photo-bleaching. After the fixation, the tissue was removed and stored in ice cold sterile PBS with Pen-Strep for immunofluorescence staining and subsequent microscopy.

For human tissues, duodenal tissue from adult human patients (n=3 patients), who did not have any prior history of chronic intestinal dysmotility, that was removed by Whipple procedure was obtained. A colonic sample from a pathologically normal colonic resection from an adult donor suffering from colon carcinoma who similarly did not have prior history of chronic intestinal dysmotility was also obtained. The resected tissue was placed in ice cold Opti-MEM medium (Invitrogen) containing Pen-Strep (Invitrogen). The mucosal and sub-mucosal tissue was dissected out in the medium under light microscope and the muscularis layer containing myenteric plexus tissue was obtained. The tissue was laid out between two glass slides and fixed overnight in ice cold 4% PFA after which it was removed and stored in ice cold sterile PBS with Pen-Strep for immunofluorescence staining, optical clarification and subsequent microscopy.

Immunohistochemistry:

For murine tissue: The fixed LM-MP tissue was washed twice in ice-cold PBS in the dark at 16° C. The tissue was then incubated in blocking-permeabilizing buffer (BPB; 5% normal goat serum with 0.3% Triton-X) for 1 hour. While staining for antibodies that were mouse monoclonal, 5% normal mouse serum was added to the BPB. The tissue was then removed from the BPB and was incubated with the appropriate primary antibody at the listed concentration (Table 1) for 48 h at 16° C. in the dark with shaking at 55 rpm. Following incubation with primary antibody, the tissue was washed three times (15-min wash each) in PBS at room temperature in the dark. The tissue was then incubated in the appropriate secondary antibody at room temperature for 1 hour while on a rotary shaker (65 rpm). The tissue was again washed three times in PBS at room temperature, counterstained with DAPI to stain the nuclei, overlaid with Prolong Antifade Gold mounting medium, cover-slipped, and imaged.

Colchicine treatment: For CGRP immunostaining, mice were injected with Colchicine at a concentration of 5 mg/Kg body weight 16 hours (overnight) before they were sacrificed. The mice were housed singly during this time and adequate gel packs were provided. Food and water were provided ad libitum. On the following day, the mice were sacrificed, and their LM-MP tissues were harvested as detailed above.

For human tissue: The fixed muscularis layer containing myenteric plexus tissue was removed from ice cold PBS and incubated in blocking-permeabilizing buffer (BPB; 5% normal goat serum, 5% normal mouse serum with 0.3% Triton-X) for 4 hours. The tissue was then removed from the BPB and was incubated with the appropriate primary antibody at the listed concentration (Table 1) for 5 days at 16° C. in the dark with shaking at 55 rpm. Following incubation with primary antibody, the tissue was washed five times (15-min wash each) in PBS at room temperature in the dark. The tissue was then incubated in the appropriate secondary antibody at 16° C. in the dark with shaking at 55 rpm for 2 days. The tissue was again washed in dark for five times in PBS that contained DAPI at room temperature. After the final wash, the tissue was suspended in tissue clarification buffer CUBIC⁸¹ for 1 hour at 4° C. in the dark after which it was overlaid with Prolong Antifade Gold mounting medium, cover-slipped, and imaged. Briefly, the CUBIC optical clarification buffer was made by mixing 2.5 g of urea (25% by wt), 2.5 g of N, N, N′, N′-tetrakis (2-hydroxy-propyl) ethylenediamine (25% by wt), 1.5 g of Triton X-100 (15% by wt) in 35 ml of Distilled Water. The solution was shaken till the ingredients were dissolved and yielded a clear viscous solution.

Microscopy:

Imaging was done by using the oil immersion 63× objective on the Leica SP8 confocal microscope and by using the oil immersion 40× objective on the Olympus Fluoview 3000rs confocal microscope with resonance scanning mode. For thick tissues, such as human tissues, the Galvano mode of the Olympus Fluoview 3000rs microscope that enabled higher resolution imaging and averaging was used. Images obtained were then analyzed using Fiji (fiji.sc/).

Enumeration of Neurons:

Identification of myenteric ganglia was performed according to our pre-determined method published earlier⁷¹. Briefly, contiguous clusters of neurons were defined as a ganglia and the total numbers of neurons within these clusters were enumerated as numbers of myenteric neurons per ganglion. As a rule, clusters of 3 neurons or more were deemed to consist a ganglion and the enumeration strategy did not count extra-ganglionic neurons. At least 10 ganglia per tissue were imaged for enumeration and each group studied had n≥3 mice. Identification and enumeration of neurons and detection of co-localization was performed manually by trained laboratory personnel.

Protein Isolation and Detection:

After the LM-MP tissue was isolated, it was weighed and placed in a sterile 1.5 ml microfuge tube. 1× RIPA buffer (Cell Signaling Technology) with Halt Protease Inhibitor Cocktail (Thermo Scientific) at 5× concentration, Phosphatase Inhibitor Cocktails II and III (Sigma-Aldrich) at 2× concentrations were added to the tissue lysate buffer. Tissue was disrupted using 1.0 mm silica beads in Bullet Blender 24 (Next Advance) for 5 minutes at highest setting. The lysate was incubated at 4° C. with shaking for 30 minutes, centrifuged at 14,000 rpm for 20 minutes and the supernatant was taken and stored in −80° C. in aliquots. Protein concentration was estimated using Bradford assay solution (Biorad) following the manufacturer's protocol. For immunoblotting, 40 μg of protein was loaded per well of 4%-20% gradient denaturing gel (Biorad). Protein marker used was Precision Plus Dual Color standards (Biorad). After fractionating the proteins, they were blotted onto ImmunBlot PVDF membrane (Biorad) overnight at 4° C. at 17V for 12-16 hours. After blotting, membrane was blocked with Odyssey TBS blocking buffer (Li-Cor) for 1 hour at room temperature with shaking. Incubation with primary antibodies were carried out at 4° C. with shaking for 24 hours. Following binding, the blot was washed 4 times with TBS-T (Tris Buffered Saline with 0.5% Tween) for 15 minutes each with shaking at room temperature. Secondary antibody incubation was carried out in dark at room temperature for 1.5 hours with shaking. The blot was then washed 4 times for 15 minutes each and imaged on Odyssey CLx system (Li-Cor). Antibodies used are detailed in the Table 1.

RNA Isolation and Quantitative Detection of Specific Transcripts:

The isolated tissue was stored in RNALater Solution (Ambion). RNA was isolated using RNeasy Mini Kit (Qiagen) following manufacturer's protocol. RNA quantification was carried out using Epoch Microplate Spectrophotometer (BioTek). cDNA synthesis was carried by SuperScript IV VILO Master Mix (Invitrogen). Quantitative Real-time PCR was carried out using Taqman Gene Expression Master Mix (Applied Biosystems) and Roto-Gene Q (Qiagen). The probes used are listed in Table 1.

Single Cell RNA Sequencing and Analyses:

Single cell preparation from adult murine ileal tissues: Ileal tissues from two 6-month old adult male littermate C57/BL6 wildtype mice were isolated by peeling as previously described. The tissues were then dissociated in Digestion Buffer containing 1 mg/ml Liberase (Sigma-Aldrich) in OptiMEM. Tissues from mouse 1 were dissociated in the Digestion buffer containing Liberase TH and tissues from mouse 2 were dissociated in the Digestion buffer containing Liberase TL. Dissociation was performed at 37° C. for 30 minutes on a rotary shaker, after which the cells were centrifuged at 200 g for 7 minutes, and the pellet was resuspended in ice cold sterile PBS. The cell suspension was passed through a 40 μm cell sieve and the resulting filtered cell suspension was again centrifuged at 200 g for 7 minutes. This process of cell centrifugation and filtration was repeated two more times, after which the cells were resuspended in 1 ml ice cold sterile PBS. The repeated steps of serial cell washes and filtration removed clumps and debris and the viability of the resulting cell suspension was estimated to be >90% using Trypan Blue dye test. The cells were then processed through 10× Genomics Chromium V2.0 system according to the manufacturer's suggested workflow. The processing was done at the GRCF Core Facility at the Johns Hopkins University. The pooled libraries were sequenced on an Illumina HiSeq 2500 to an average depth of 3.125×10⁸ reads per sample library. The sequencing was performed at the CIDR core facility at the Johns Hopkins University.

Pre-processing of FASTQs to Expression Matrices: FASTQ sequence files were processed following a Kallisto Bustools workflow compatible with downstream RNA velocity calculations [kallisto==0.46.1, bustools==0.39.3]⁸². References required for pseudo-alignment of transcripts were obtained using the get_velocity_files (functionality of BUSpaRSE (github.com/BUStools/BUSpaRse)), with “L=98” for 10× Genomics v2.0 sequencing chemistry. Reads were pseudo-aligned to an index built from Ensembl 97 transcriptome annotation (Gencode vM22; GRCm38). Across two samples processed, a total of 578,529,125 reads were successfully pseudo-aligned. Barcodes within a Hamming distance of one to known 10× Genomics v2.0 barcodes were corrected. Reads were classified as “spliced” or “unspliced” by their complement to the target list of intronic sequences and exonic sequences, respectively, and subsequently quantified separately into expression count matrices. Spliced counts are used for all analyses.

Single cell gene expression analysis: scRNA-seq count matrices were analyzed using Monocle3. 11,123 high-quality cells were identified as meeting a 200 UMI minimum threshold with a mitochondrial read ratio of less than 20%; droplets that did not meet these criteria were excluded from the analysis. Mitochondrial counts were determined as the sum of reads mapping to 37 genes annotated to the mitochondrial genome. All genes with non-zero expression were included for downstream analysis. Raw counts were first scaled by a cell-size scaling factor and subsequently log 10 transformed with a pseudo-count of 1. Normalized values are used in place of raw counts unless otherwise noted.

Prior to UMAP dimensionality reduction, batch effects between the two biological replicates were assessed and corrected via the mutual nearest neighbors (MNN) algorithm as implemented by Batchelor in Monocle3 (50 principal components, with default k=20)⁸³. 15 clusters of cells in the UMAP embedding were identified by Leiden community detection (resolution=1e−5, number of iterations=5). 30 marker genes for each cluster were identified based on greatest pseudo R² values and used for supervised annotation of cell types by searching UniProt, Allen Cell Atlas and through literature search with Pubmed.

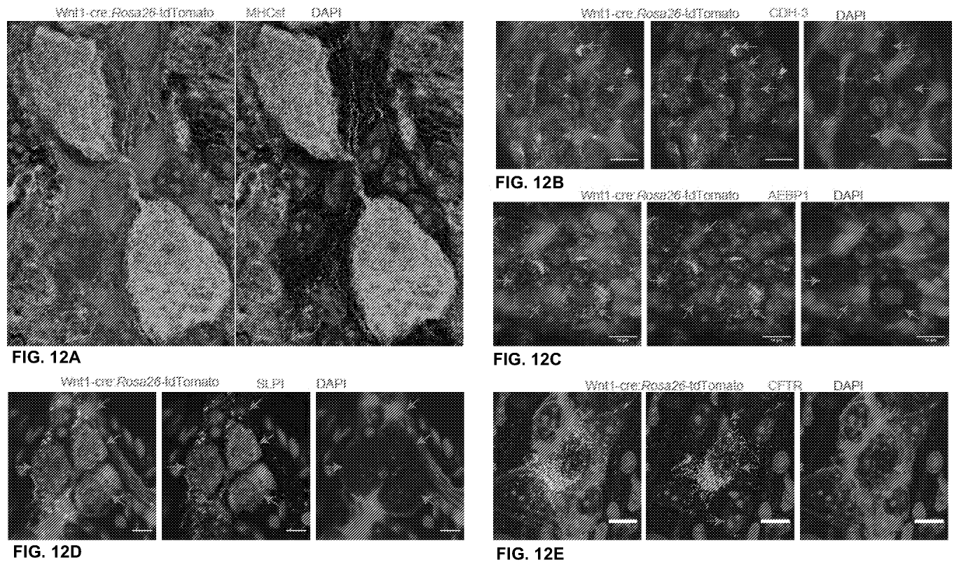

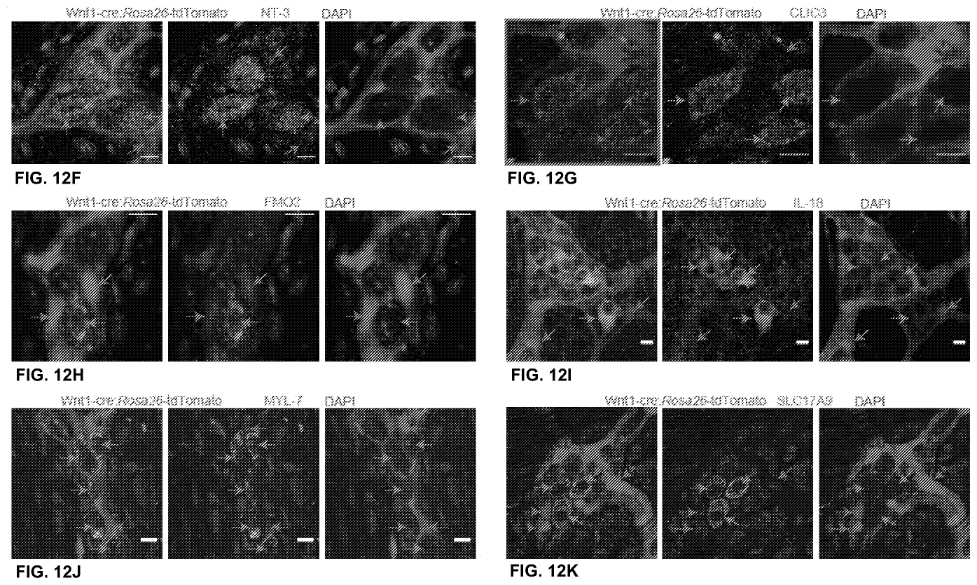

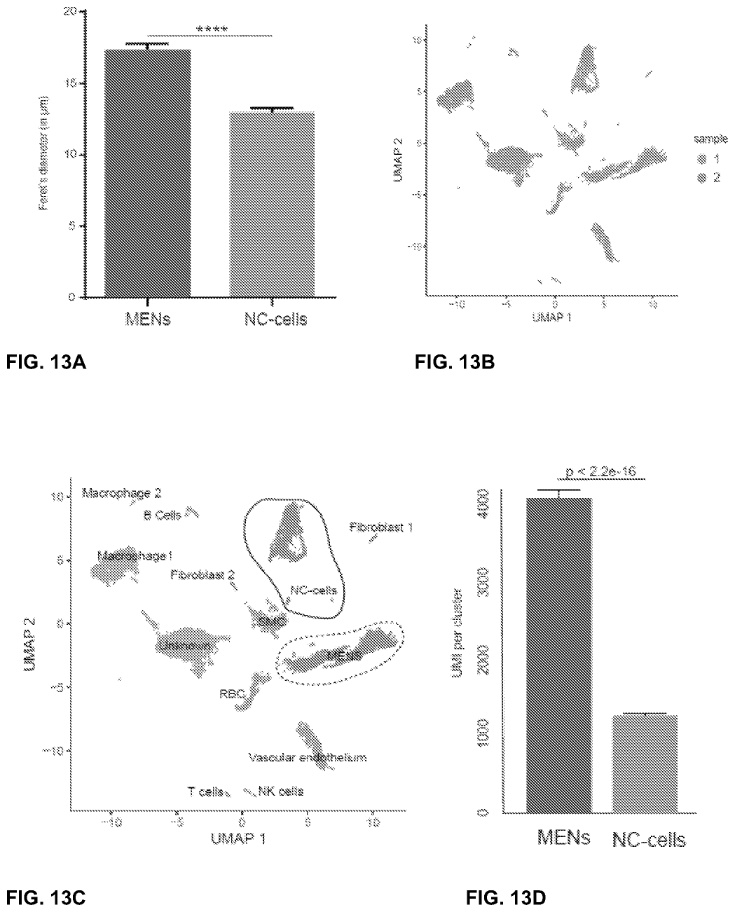

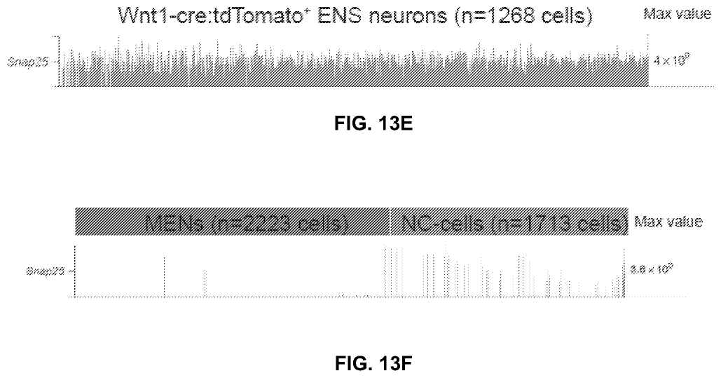

NC-derived cell clusters were identified by expression of NC markers Ret and Sox10. MEN cluster was identified by its expression of CGRP-coding Calcb, Met, and Cdh3. The pan-MENs protein marker MHCst was identified by labeling with an antibody S46 which labels all members of the MHCst family³⁸. Since the antibody does not identify a single gene product, MHCst immunostaining could not be used to identify a specific gene marker for use in the annotation of the MEN cluster. For further analysis into the MENs population, the full LM-MP dataset was subset to include only the 2,223 cells annotated as such. These cells were re-processed as above, but with a reduced PCA dimensionality of k=20 as input for the UMAP embedding. 5 clusters of cells in the UMAP embedding were identified by Leiden community detection (k=10, resolution=5e−4). The two samples showed a similar distribution of cells across the various cell clusters (FIG. 12A). The MEN cluster had a significantly higher mean UMI count/cell compared to the NC-derived cell cluster (Mean UMI±S.E.M. of UMI/cell: MENs: 4045.6±113.4; NENs: 1247.9±38.9; t=23.34, df=2727.8, p<2.2e−16; Welch two sample t-test; Fig S4D). Total UMI per cell is correlated directly with cell size⁸⁴. A significantly higher UMI/cell in MENs compared to NENs suggests that MENs would have significantly larger cell size compared to NENs, which was confirmed in FIG. 13A.

Pattern discovery and ProjectR analyses: Pattern discovery was utilized to identify sets of co-expressed genes that define cell-type specific transcriptional signatures. The normalized expression matrix was decomposed via non-negative matrix factorization (NMF) as implemented in the R package NNLM (github.com/linxihui/NNLM), with k=50 and default parameters. Cell weights for each pattern were grouped by assigned cell-type and represented by heatmap. Pattern vectors were hierarchically clustered by a Euclidian distance metric, implemented in ComplexHeatmap (github.com/jokergoo/ComplexHeatmap)⁸⁵. These patterns were then tested on the bulk RNA-Seq expression matrix for the Human Obstructed Defecation study⁵⁷ (GSE101968). The log 2 expression (log 2(rpkm+1)) from this study was projected onto the NMF patterns using projectR⁶¹. Students' t tests were performed on the projection weights from the Control and OD groups to test for differences between them.

Whole Gut Transit Time Analyses:

Whole-gut transit time (WGTT) for every mouse was analyzed by the method using the carmine red protocol⁷¹. Mice were placed in individual cages and deprived of food for 1 hour before receiving 0.3 mL 6% (wt/vol) carmine solution in 0.5% methylcellulose by oral gavage into the animal's stomach. The time taken for each mouse to produce a red fecal pellet after the administration of carmine dye was recorded in minutes. The experiment was terminated at 210 minutes post-gavage and the WGTT of any mice that did not expel the red dye at the termination was marked at the value of 210 min. The mean difference in whole gut transit time (in minutes) between both the Ret^{+/+} and Ret^{+/-} mice cohorts, and the GDNF and Saline-treated Control cohorts were analyzed statistically.

In Vivo Injections:

GDNF injection: Similar to prior report that gave sub-cutaneous injections of GDNF to post-natal mice-, 6 littermates, 10 day old (P10) male Wnt1-cre:Rosa26-tdTomato mice were taken and divided into two subgroups, GDNF and Control. Each mouse in the GDNF group was injected sub-cutaneously with 50 μl of 2 mg/ml of GDNF (Peprotech Catalogue #: 450-44) every other day, while the Control group was injected with 50 μl of sterile saline. The mice were given 5 doses and then sacrificed on P20, after which their LM-MP tissues were isolated as detailed above. The tissues were then immunostained with antibodies against HuC/D and imaged. In a separate experiment, adult (P60) mice were also injected sub-cutaneously with GDNF (100 μl of 100 μg/ml of GDNF). The mice were given 5 doses over a course of 10 days and then sacrificed on P70, after which their LM-MP tissues were isolated as detailed above. For studying the effect of GDNF on aging mice, two cohorts of 17-month-old male C57BL/6 mice (n=5 mice/cohort) were obtained from the aging colony of the National Institute of Aging. Before the start of dosing, the whole gut transit time was assayed. The animals were then injected daily sub-cutaneously either with 100 μl of saline (Control) or 100 μl of GDNF (500 μg/ml) for 10 consecutive days, after which the mice were sacrificed and their LM-MP tissues were isolated as detailed above. The tissues were then immunostained with antibodies against HuC/D and imaged.

HGF injection: Similar to prior report that gave sub-cutaneous injections of HGF to post-natal mice, we took 6 littermate 10 day old (P10) male Wnt1-cre:Rosa26-tdTomato mice and divided into two subgroups, HGF and Control. Each mouse in the HGF group was injected sub-cutaneously with 100 μl of 2 mg/ml of HGF (Peprotech Catalogue #:315-23) every other day, while the saline group was injected with 100 μl of sterile saline. The mice were given 5 doses and then sacrificed on P20, after which their LM-MP tissues were isolated as detailed above. The tissues were then immunostained with antibodies against HuC/D and imaged.

Statistics:

Data was analyzed using Graphpad Prism 8.3.1 and R using Unpaired Students t-test, Simple Linear Regression, and Ordinary One-Way ANOVA.

Data:

All raw data are provided in Supplementary Table 2-22. We imaged at least 10 ganglia per tissue for our enumeration and each group studied had n≥3 mice. Raw single cell RNA sequencing data is archived on the NCBI GEO server and can be accessed under the accession number GSE156146.

TABLE 1
Primary Antibodies and Antisera

| Antigenic | | | |
|---|---|---|---|
| target | Species | Company | Dilution |
| HuC/D | Human | Human antisera ANNA1 | 1:750-1:1000 |
| HuC/D | Rabbit | Abcam | 1:500 |
| NOS1 | Rabbit | Invitrogen | 1:200 |
| CGRP | Rabbit | Immunostar | 1:500 |
| MET | Goat | R&D systems | 1:250 |
| MHCst (S46) | Mouse | DHSB Iowa | 1:250 |
| RET | Rabbit | Abcam | 1:500 |
| CDH-3 | Rabbit | Abcam | 1:500 |
| RFP | Rabbit | Rockland | 1:500 |
| GFAP | Rabbit | DAKO | 1:1000 |
| β ACTIN | Mouse | MP Biomedicals | 1:10,000 |
| HGF | Rabbit | Abcam | 1:500 |
| GDNF | Rabbit | Abcam | 1:500 |
| GFP | Chicken | Aves | 1:1000 |
| SLPI | Rabbit | Abbexa | 1:200 |
| AEBP1 | Rabbit | Biorbyt | 1:250 |
| CLIC3 | Rabbit | Proteintech | 1:250 |
| CFTR | Mouse | CF Foundation | 1:500 |
| SMO | Rabbit | Proteintech | 1:250 |
| FMO2 | Rabbit | Biorbyt | 1:250 |
| SLC17A9 | Rabbit | Proteintech | 1:200 |
| IL-18 | Rabbit | Abcam | 1:400 |
| NT-3 | Rabbit | Chemicon | 1:200 |
| MYL-7 | Rabbit | Abcam | 1:100 |
| SNAP-25 | Rabbit | ProteinTech | 1:200 |

Secondary Antibodies

| Reactivity and | | | |
|---|---|---|---|
| fluorophore | Species | Company | Dilution |
| Anti-Rabbit 488 | Goat | Invitrogen | 1:500 |
| Anti-Rabbit 647 | Goat | Invitrogen | 1:500 |
| Anti-Rabbit 647 | Donkey | Invitrogen | 1:500 |
| Anti-Chicken 647 | Donkey | Invitrogen | 1:500 |
| Anti-Chicken 488 | Donkey | Invitrogen | 1:500 |
| Ant-Mouse 647 | Donkey | Invitrogen | 1:500 |
| Anti-Mouse IRdye 680RD | Donkey | Li-Cor | 1:10,000 |
| Anti-Rabbit IRdye 800CW | Donkey | Li-Cor | 1:10,000 |

TaqMan Probes

| Probe ID | Target | Company |
|---|---|---|
| Mm01135184_m1 | Hgf | Invitrogen |
| Mm00446968_m1 | Hprt | Invitrogen |

Results

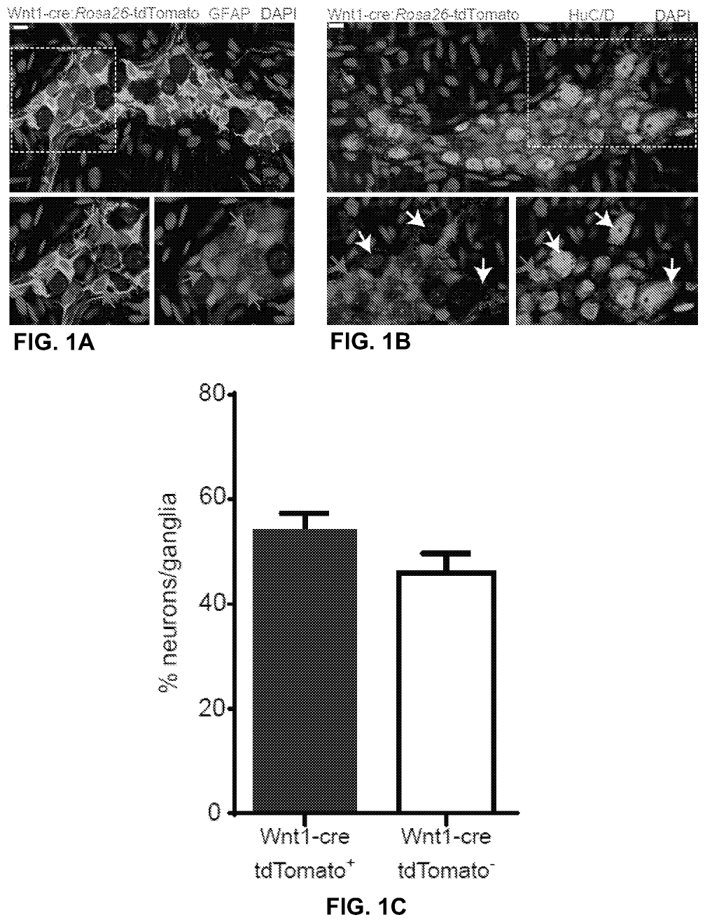

Only half of all mid-age adult enteric neurons are derived from the neural crest. Small intestinal longitudinal muscle-myenteric plexus (LM-MP) from adult (post-natal day 60; P60) Wnt1I-cre:Rosa26-tdTomato mice, was analyzed in which tdTomato is expressed by all derivatives of Wnt1⁺ NC-cells¹⁸. In these tissues, while GFAP, a glial marker, was always co-expressed with tdTomato (FIG. 1A), tdTomato-expression was absent in almost half of all myenteric neurons expressing the pan-neuronal marker HuC/D (tdTomato⁺ neurons: 53.5±3.8 SEM; tdTomato neurons: 45.92±3.8 SEM; n=53 ganglia from 5 mice, p=0.16; FIGS. 1B, 1C). In these lineage-traced mice, myenteric ganglia were found to contain tdTomato^{high} and tdTomato^{low} neurons and due care was taken to image subsets (FIG. 10A). Both tdTomato^{high} and tdTomato^{low} neurons were classified as tdTomato⁺ and only neurons that did not show any tdTomato-expression were classified as tdTomato⁻ neurons. Additional confirmation was provided by an analogous Wnt1-cre:Hprt-tdTomato lineage-traced mouse line, in which tdTomato was expressed from the Hprt locus in a Wnt1-cre-dependent manner (Hprt locus is X-inactivated in females, hence only adult male mice were used)¹⁹; and by the Pax3-cre:Rosa26-tdTomato lineage-traced mouse line, where Rosa26-tdTomato driven by the Pax3-cre transgene labels the derivatives of the neural tube and pre-migratory NC (FIGS. 10B, 10C)²⁰. Similar lack of reporter expression was previously observed in adult myenteric neurons from Sox10-cre NC-lineage-traced mice, confirming the observations of the presence of a significant proportion of non-NC-derived neurons in the adult ENS¹³.

Lineage-tracing confirms a mesodermal derivation for half of all adult myenteric neurons. Alternative sources of enteric neurons proposed previously include the ventral neural tube¹⁵, and the pancreatic endoderm¹⁴, but the interpretation of these studies was limited by the lack of robust lineage markers¹⁶. Brokhman et al¹⁴ found evidence of labeled neurons in inducible Pdx1-cre, Foxa2-cre, and Sox17-cre lineage-traced mouse lines and inferred their derivation from pancreatic endoderm. However, in a Pdx1-cre lineage-traced mouse line, many neuroectoderm-derived neurons of the central nervous system have also been previously shown to be derived from Pdx1-expressing cells²¹, suggesting that Pdx1 is not an exclusive endodermal marker. Foxa2 is expressed by mesoderm-derived cells in the heart²² and Sox17 also labels mesoderm-derived cells, especially cells of the intestinal vasculature²³⁻²⁶. It was therefore hypothesized that the embryonic mesoderm may be the true developmental source of the non-NC enteric neurons.

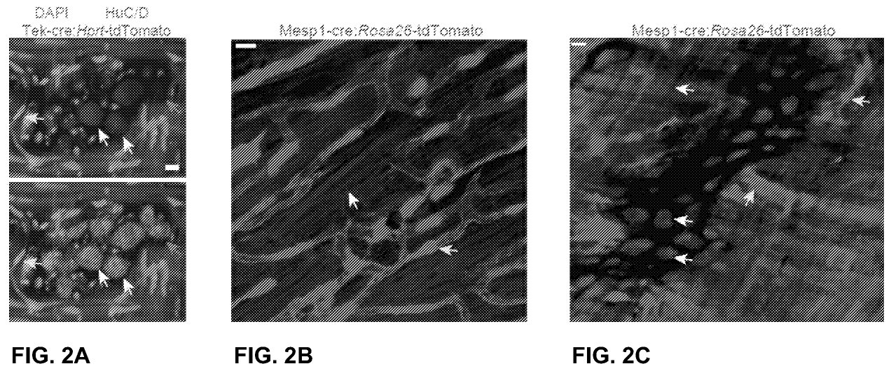

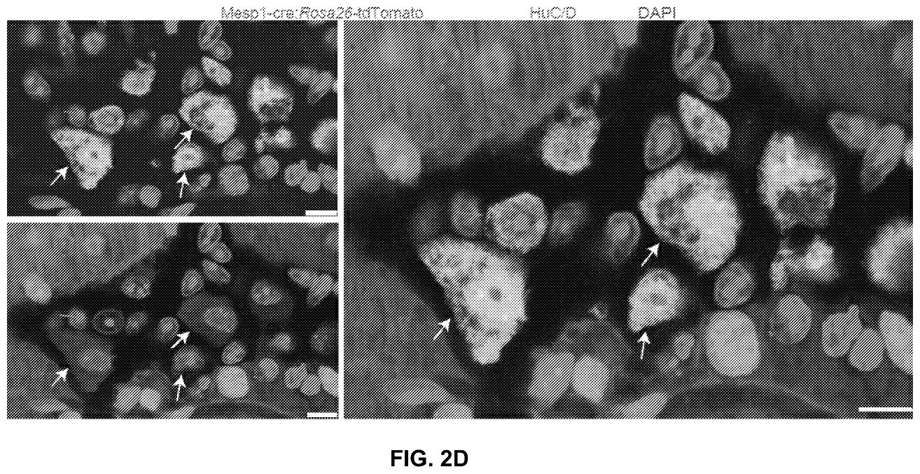

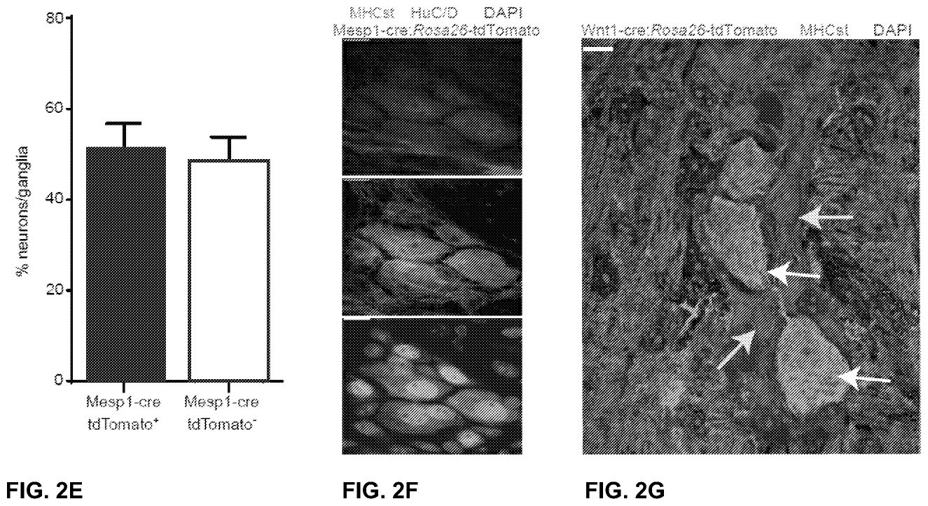

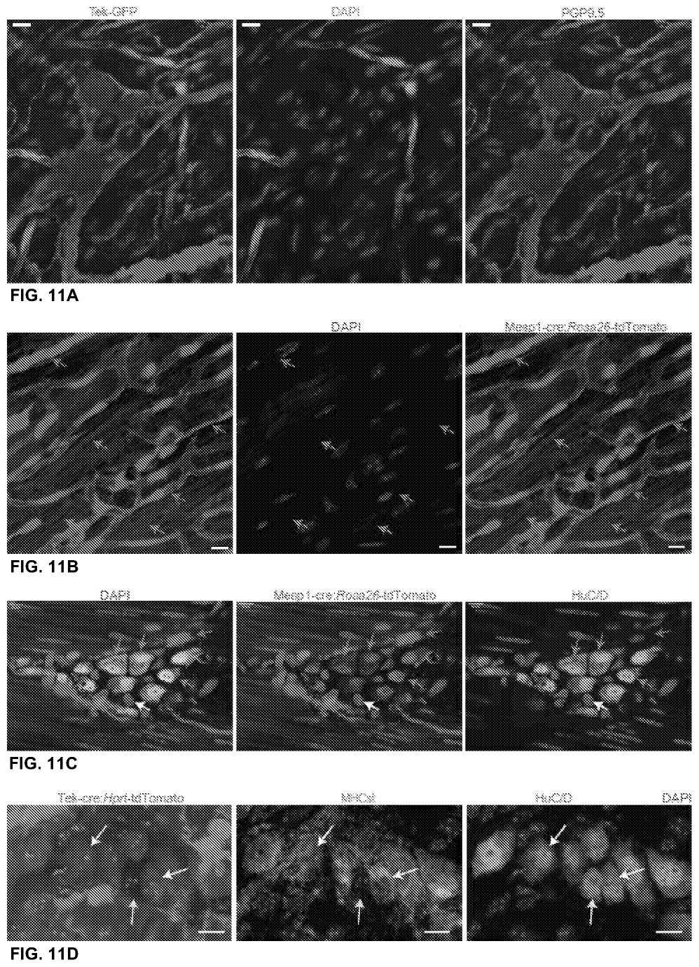

Mesoderm-derived cells during embryogenesis express Tek²² and analysis of LM-MP tissues from adult male Tek-cre:Hprt-tdTomato lineage-traced mice revealed the presence of a population of tdTomato⁺ neurons (FIG. 2A). Since Tek is not expressed in the adult ENS (FIG. 11A), the presence of Tek-derived adult myenteric neurons provided evidence of their mesodermal origin. Mesoderm posterior 1 or Mesp1 was used as a definitive developmental marker for the embryonic mesoderm²⁷⁻³¹ to confirm the mesodermal derivation of the non-NC derived adult enteric neurons. Reporter expression in this mouse line has been previously studied in cardiac development, which was confirmed by observing its expression in the adult heart tissue (FIG. 2B).

Expression of tdTomato in both Tek-cre and Mesp1-cre mice was less pronounced in cardiac myocytes (FIGS. 2B, 11B) and myenteric neurons (FIGS. 2A, 2C) as compared with vascular cells, which reflects the variable expression of the reporter gene in both the neural crest- and mesoderm-lineage specific transgenic mouse lines (FIGS. 2B, 2C, 10A, 11B, 11C). Variable expression of CAG and other pan-cellular promoters in various cell-types and during processes of maturation have previously been reported³²⁻³⁵. In addition, an earlier study by Agah et al showed differing degrees of CAG-driven LacZ reporter activation in a cardiac-specific transgenic cre mouse line, which were unrelated to copy number, suggesting insertional and positional effects or, potentially, differential methylation³⁶. These reports are consistent with the observations here and could help explain the observed variable expression of CAG and CMV promoter-driven reporter genes in this study.

Analysis of small intestinal LM-MP from P60 Mesp1-cre:Rosa26-tdTomato lineage-traced mice (FIGS. 2D, 11C) showed that tdTomato-expression was observed in about half of all myenteric neurons (tdTomato⁺ neurons: 48.5±5.24 SEM; tdTomato⁻ neurons: 51.5±5.24 SEM; n=31 ganglia from 3 mice, p=0.68) (FIG. 2E). Because of the variable expression of the reporter gene in the transgenic mice described above, Mesp1-cre:Rosa26-tdTomato⁺ neurons were also labeled with the S46 antibody, which was raised against the slow tonic myosin heavy chain protein MHCst derived from avian embryonic upper leg muscle, and is expressed exclusively by mesoderm-derived cell populations (FIG. 2F)³⁷⁻⁴⁰. MHCst immunostaining was exclusively observed in all Tek-cre:Rosa26-tdTomato⁺ MENs (FIG. 11D) and Wnt1-cre:Rosa26-tdTomato non-expressing non-NC lineage neurons. Thus, both lineage tracing and protein biomarker expression provides strong support for their mesodermal origin (FIG. 2G, FIG. 12A). Along with the observations on Wnt1-cre:Rosa26-tdTomato mice (FIG. 1C), the results indicate that these two distinct lineages together account for all the adult small intestinal myenteric neurons.

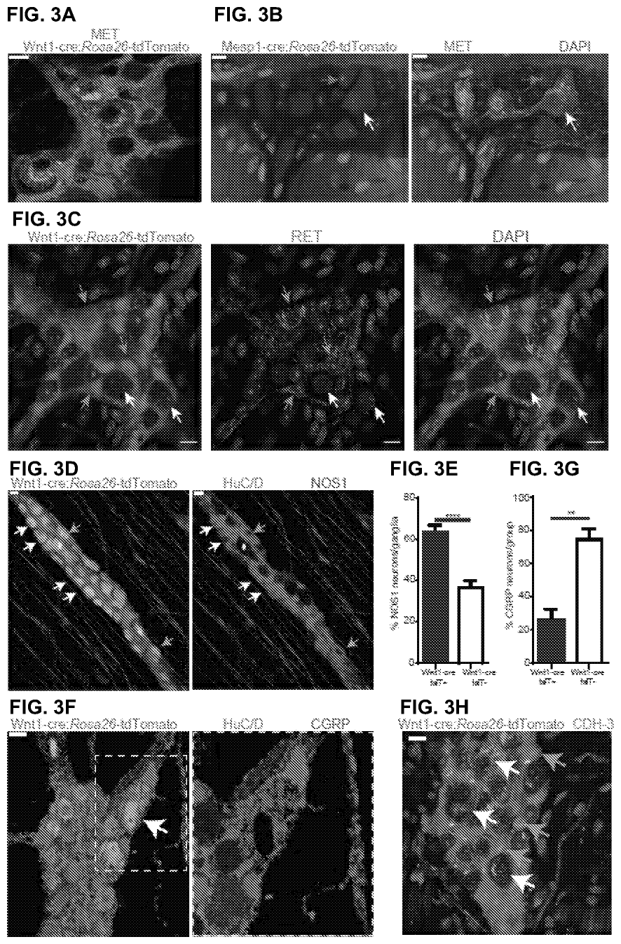

The proteins RET, a receptor tyrosine kinase that transduces GDNF signaling in NC-derived enteric neuronal precursors, and MET, a receptor for hepatocyte growth factor (HGF), are expressed by different subsets of adult myenteric neurons⁴¹. MET is classically expressed by mesoderm-derived cells⁴², and by using immunostaining of small intestinal LM-MP tissues from Wnt1-cre:Rosa26-tdTomato and Mesp1-cre:Rosa26-tdTomato mice, it was found that the expression of MET was restricted to a sub-population of adult MENs (FIGS. 3A, 3B). By contrast, RET expression was confined to NENs (FIG. 3C).

It was then studied whether MENs and NENs differed phenotypically. In the adult murine ENS, inhibitory enteric motor neurons express the nitric oxide-producing enzyme nitric oxide synthase 1 (NOS1); and enteric sensory neurons, called intrinsic primary afferent neurons (IPANs) express the neuropeptide calcitonin gene related peptide (CGRP)⁹⁴¹. Analyses of NOS1 and CGRP expression in the LM-MP from P60 Wnt1-cre:Rosa26-tdTomato mice showed that while tdTomato⁺ neurons account for the majority of NOS1⁺ inhibitory neurons (NOS1⁺ tdTomato⁺: 63.42±3.33 SEM; NOS1⁺ tdTomato-: 36.58±3.33 SEM; n=32 ganglia from 3 mice, p<0.0001;

FIGS. 3D, 3E), tdTomato⁻ neurons account for the majority of CGRP⁺ neurons (CGRP⁺ tdTomato⁻: 74.36±2.18 SEM; CGRP⁺ tdTomato⁺: 25.64±2.18 SEM; n=30 ganglia from 3 mice, p<0.0001; FIGS. 3F, 3G). These results are in keeping with a previous report that show low expression of NOS1 and abundant expression of CGRP by MET⁺ neurons⁴¹, previously not known to be of a different lineage. The cell size of MENs was significantly larger than that of NENs (n=143 neurons/group; Feret diameter (μm) MENs: 17.47±0.36 SEM, NENs: 13.05±0.23 SEM,p<0.0001; FIG. 13A), which is consistent with their putative role as IPANs, which are also known consist of large diameter neurons⁴³. A sensory role for MENs is also supported by their specific expression of Cadherin-3 (CDH3, FIGS. 3H, 12B), which marks a sub-population of mechanosensory spinal neurons⁴⁴.

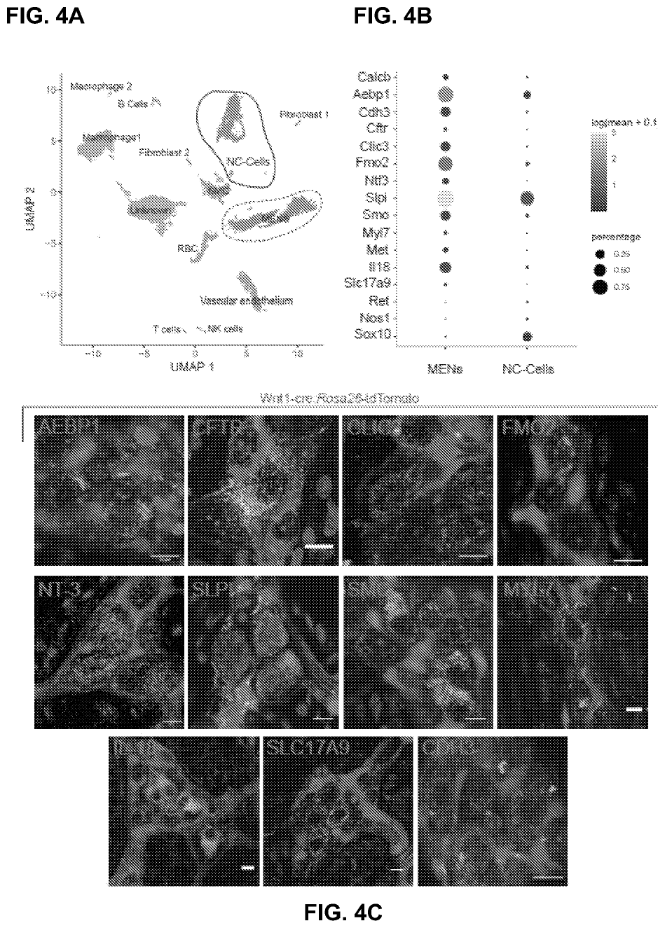

An expanded molecular characterization of MENs using unbiased single cell RNA sequencing (scRNAseq)-based analyses. The separate nature of the MENs and NENs was further ascertained by scRNAseq-based analyses on all cells isolated from ileal LM-MP. Unlike other studies that analyzed either FACS-sorted Wnt1-cre:Rosa26-tdTomato⁺ ENS-cells or used classical NENs markers to drive identification of enteric neurons⁴, agnostic clustering of the scRNAseq data from tissues of two 6-month-old adult male C57BL/6 wildtype mice was performed (FIGS. 13B, 13C). The cluster of NC-derived cells was identified by exclusive expression of canonical NC markers Ret and Sox10; and the MENs cluster by its co-expression of the genes Calcb (CGRP), Met, and Cdh3 (FIGS. 4A; 13B, 13C). With cells from both samples pooled together, 1,713 NC-derived cells were compared with 2,223 MENs.

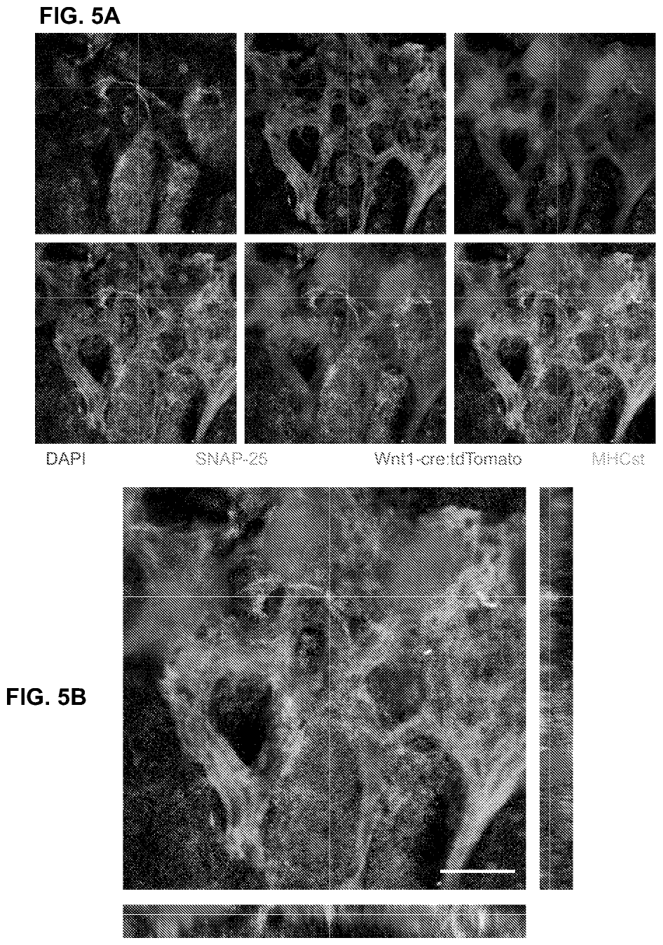

Examination of the MENs cluster yielded additional MENs-specific marker genes Slpi, Aebp1, Clic3, Fmo2, Smo, Myl7, and Slc17a9, whose expression by adult enteric neurons has not been previously described (FIG. 4B). In addition, the MEN cluster was also enriched in previously characterized ENS markers, such as Ntf3 and Il18^{2,47} (FIG. 4B). Exclusive expression of 1118 by MENs, which has recently been shown to regulate mucosal immunity², indicates a potential immunomodulatory function. The MEN-specificity of these markers was validated using immunochemical analyses (FIGS. 4C; 12B—12K). In addition to these MEN-specific genes, the expression of the pre-synaptic gene Snap25, a component of the SNARE complex, has been reported in all NC-derived Wnt1-cre:tdTomato⁺ enteric neurons (FIG. 13E) and in a subset of human ENS neurons⁴⁵′4, while it is not detected in the MEN cluster in the analyses (FIG. 13F). While the SNAP-25 expressing neurons did express tdTomato in the myenteric ganglia of a Wnt1-cre:Rosa26-tdTomato transgenic mouse, they did not express the MENs-marker MHCst, suggesting the NENs-specific expression of this canonical marker for synaptic neurons (FIG. 5).

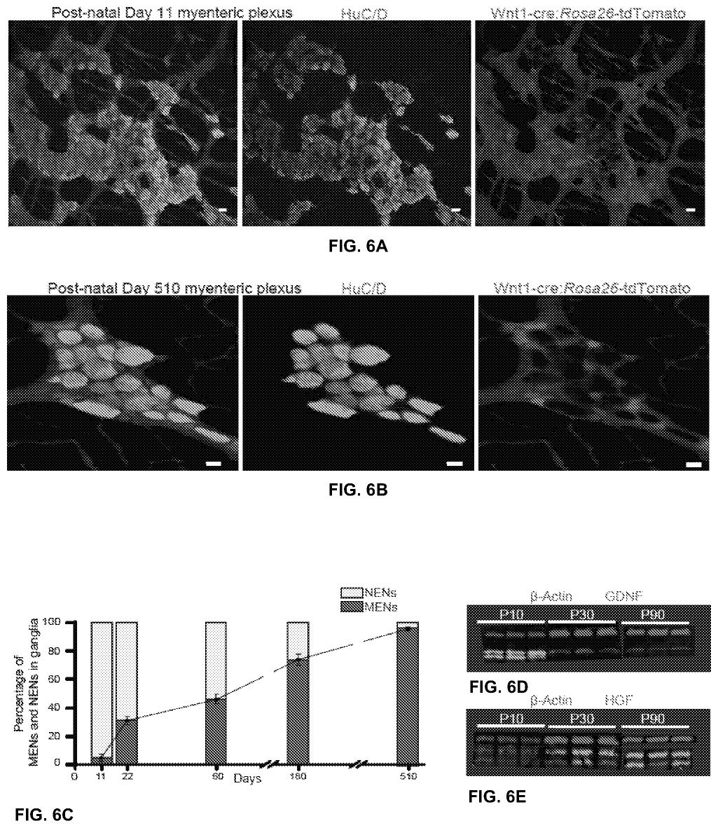

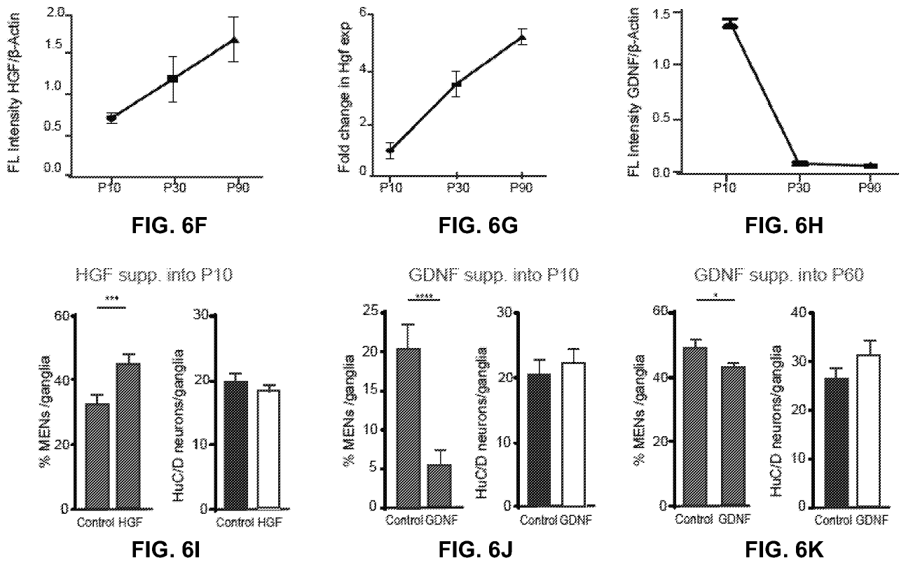

The proportion of mesoderm-derived neurons expands with age to become the dominant population in the aging ENS. Since the ENS at birth consists solely of NENs¹³, the birth-date and eventual expansion of the MEN lineage was studied in the post-natal gut. Using Wnt1-cre:Rosa26-tdTomato mouse line, the tdTomato⁻ MENs in LM-MP was enumerated at different ages and a significant age-associated increase was found in MENs proportions (F=296.0, DFn, DFd=1, 250; p<0.0001) (FIGS. 6A, 6B, 6C). At P11, MENs were found only in few isolated myenteric ganglia (FIG. 6A) and together represented only˜5% of all myenteric neurons (n=34 ganglia from 3 mice), suggesting that they originate shortly before this age. The proportion of MENs in myenteric ganglia rises sharply thereafter: by P22, they account for ˜30% of all neurons (n=45 ganglia from 3 mice); and at P60 they represent ˜45% of all neurons (n=37 ganglia from 3 mice). By P180, MEN comprise 75% of the ENS (n=60 ganglia from 6 mice) and at very old age, P510, the ENS is populated almost exclusively by MENs (FIG. 6B, n=67 ganglia from 3 mice).

The proportions of NENs and MENs in the myenteric ganglia that can be used as a biomarker for deducing ENS age, as a healthy ENS dominant is NENs would be juvenile, one with equal proportions would be adult, and an aging ENS is dominated by MENs.

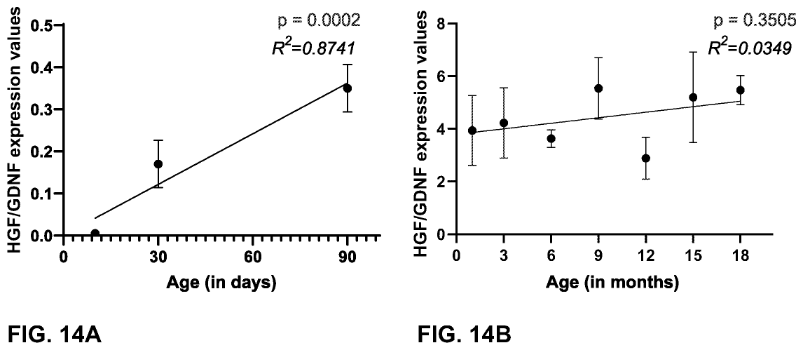

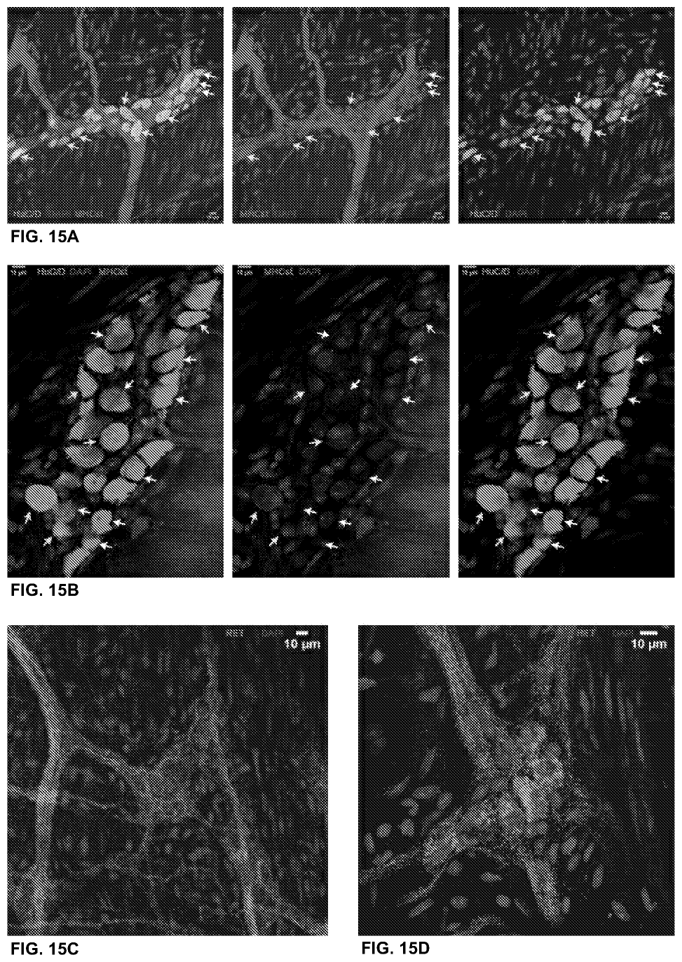

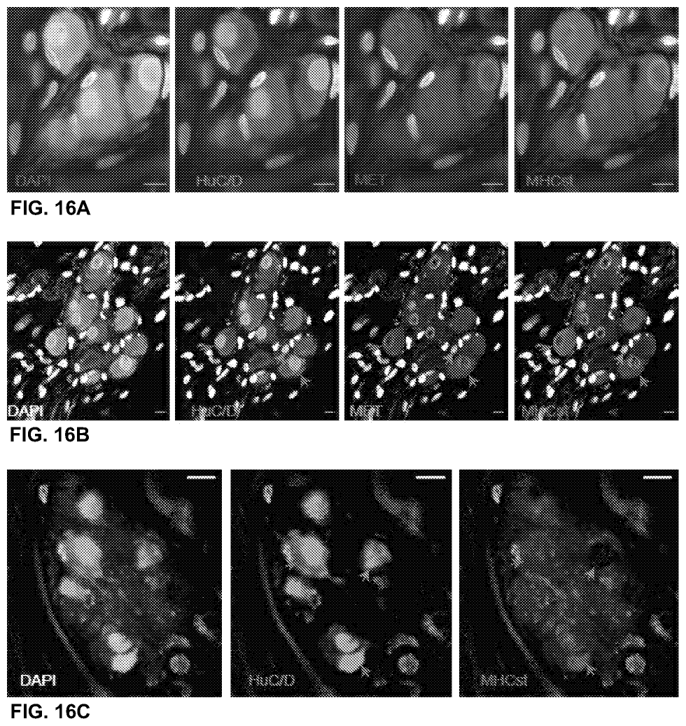

GDNF and HGF levels regulate the populations of the neural crest-derived and the mesoderm-derived neurons, respectively. GDNF-RET signaling is responsible for proliferation and neurogenesis from NC-derived ENS precursor cells during development as well as for the survival of Ret-expressing enteric neurons⁴⁹⁻⁵². Similarly, HGF signaling has been shown to be essential for the proliferation of mesoderm-derived cells⁵³. Since the expression of the HGF receptor MET and the GDNF receptor RET is exclusive to MENs and NENs respectively, the correlation between age and levels of HGF and GDNF in LM-MP was studied (FIGS. 6D, 6E). It was found that while HGF expression increases progressively between P10 and P90 ages (n=3 mice/age-group, Protein levels: F=8.820, DFn, DFd=1, 7, p=0.02; mRNA levels: F=36.98, DFn, DFd=1, 16, p<0.0001) (FIGS. 6F, 6G); in agreement with a previous report⁵³, GDNF expression is markedly reduced between the P10 and the P30 ages and remains reduced thereafter up to the P90 age (n=3 mice/age-group, F=6.821, DFn, DFd=1, 7, p=0.0348) (FIG. 5H). The ratio of HGF to GDNF expression in ileal LM-MP tissue shows significant increase from the age of P10 to P90 in our data (FIG. 14A), and HGF expression is consistently more than GDNF expression in the full thickness small intestinal tissue from adult and aging mice (FIG. 14B).

Since HGF tissue levels increase with age, it was hypothesized that increasing HGF signaling drives the expansion of MENs. HGF administration to cohorts of P10 Wnt1-cre:Rosa26-tdTomato mice over 10 days promoted an increase in the tdTomato⁻ MENs population in P20 mice to levels previously observed in P60 mice, while tissues from saline-treated control mice exhibited a MENs:NENs ratio that is expected at P20 (n=99 ganglia from 5 mice for Controls, % MENs: 32.48±2.55 SEM; n=104 ganglia from 5 mice for HGF treatment, % MENs: 45.38±2.73 SEM; p<0.001) (FIG. 6I). Interestingly, HGF-driven ENS maturation did not alter the total numbers of small intestinal myenteric neurons (FIG. 6I).

Since GDNF tissue levels are correlated with NENs proportions, it was next hypothesized that GDNF signaling regulates NENs proportions in maturing and adult ENS. On administration of GDNF or saline to cohorts of P10 Wnt1-cre:Rosa26-tdTomato mice over 10 days^{49,54}, it was found that GDNF treatment promoted the juvenile phenotype by enhancing the proportions of tdTomato⁺ NENs and correspondingly reduced the proportion of tdTomato⁻ MENs in P20 mice to a level similar to that seen at the P10 age (FIG. 6J). At the same time, the MENs proportions in saline-treated control mice remained at a level expected of its age (Controls: n=65 ganglia from 3 mice, % MENs: 79.39±2.97 SEM; GDNF: n=58 ganglia from 3 mice, % MENs: 94.51±1.75 SEM; p<0.0001) (FIG. 6J). Similar to the observations with HGF administration, the GDNF-driven expansion of NENs and associated contraction of MENs conserved the total neuronal numbers (FIG. 6J). Next tested was GDNF's ability to reverse loss of NENs in the adult ENS and it was found that GDNF administration significantly decreased MENs and increased NENs proportions in P60 male Wnt1-cre:Rosa26-tdTomato mice compared to the saline-treated age and strain-matched control mice, while again maintaining total neuronal numbers (Controls: n=57 ganglia from 3 mice, % MENs: 49.32±2.04 SEM; GDNF: n=47 ganglia from 3 mice, % MENs: 42.91±1.12 SEM; p<0.05) (FIG. 6K).

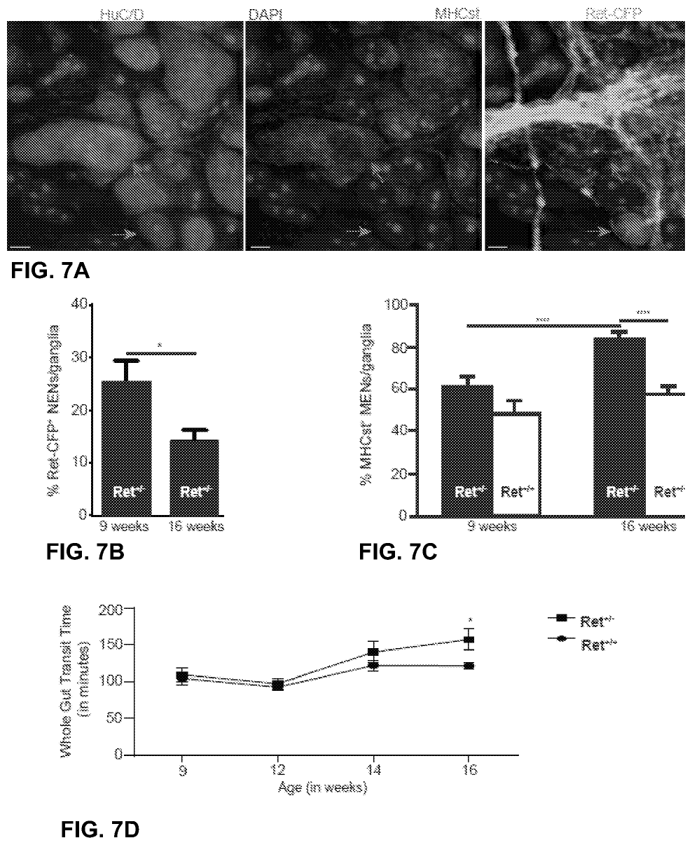

Reduced GDNF-RET signaling accelerates ENS aging to cause intestinal dysfunction. Since reduced GDNF or RET levels are associated with intestinal dysfunction in patients⁵⁵, it was hypothesized that alterations in GDNF-RET signaling unrelated to those seen with normal aging, would cause dysfunction. To test this hypothesis, lineage proportions and intestinal function were studied in a mouse model of reduced RET signaling. Ret-null heterozygous mice, which have been previously used to study the effect of reduced RET signaling in the adult ENS, have normal ENS structure but altered gut physiology⁴⁹. A similar mouse model with a Ret^{CFP} allele has a CFP reporter inserted at its Ret locus rendering it null⁵⁶ and in these mice, it was confirmed that the NENs marker Ret-CFP, and the MENs marker MHCst were expressed by different neuronal subpopulations (FIG. 7A). Using the adult Ret^{+/CFP} (or Ret^{+/-}) mice, the effect of partial Ret loss on ENS lineages was tested at two adult ages: 9 weeks (˜P60) and 16 weeks (˜P110). Ret^{+/-} mice showed a significant reduction in the proportions of Ret-CFP⁺ NENs (9 weeks: n=37 ganglia from 3 mice, % Ret-CFP⁺ NENs: 25.24±4.2 SEM; 16 weeks: n=86 ganglia from 5 mice, % Ret-CFP⁺ NENs: 14.02±2.24 SEM; p<0.05) and a corresponding significant increase in the proportions of MHCst⁺ MENs with age, while control Ret^{+/+} mice showed no significant age-associated change in the proportions of MENs (9 weeks Ret^{+/-}: n=38 ganglia from 3 mice, 25% MHCst⁺ MENs: 62.41±3.83 SEM; 16 weeks Ret^{+/-}: n=55 ganglia from 5 mice, % MHCst⁺ MENs: 84.18±2.53 SEM, p<0.0001); (9 weeks Ret^{+/-}: n=37 ganglia from 3 mice, % MHCst⁺ MENs: 50.85±4.23 SEM; 16 weeks Ret^{+/+}: n=65 ganglia from 5 mice, % MHCst⁺ MENs: 57.77±2.73 SEM, p=0.43) (FIGS. 7B, 7C). This expedited loss of NENs in Ret^{+/-} mice confirms that depletion of endogenous RET signaling in the adult ENS accelerates the aging-associated loss of NENs.

Having previously shown that aging mice have intestinal dysmotility⁵⁷, it was tested whether the increased loss of NENs in the Ret^{+/-} ENS, concomitant with the expansion of MENs accelerated ENS aging, as evidenced by the accelerated expansion of MENs, causes an early onset of aging-associated intestinal dysmotility. Intestinal transit was studied in a cohort (n=8) of adult Ret^{+/-} mice and their littermate control (n=10) Ret^{+/+} mice over 7 weeks, between 9 and 16 weeks of age. While the 9-week old adult Ret^{+/-} mice had similar whole gut transit times (WGTT) as control Ret^{+/+} mice, WGTT between the two genotypes diverged with age. 16-week old Ret^{+/-} mice displayed significantly delayed intestinal transit compared to age-matched control Ret^{+/+} mice (WGTT (in min) Ret^{+/+}: 121.4±4.01 SEM; Ret^{+/-}: 157.3±14.62 SEM, p<0.05)(FIG. 7D).

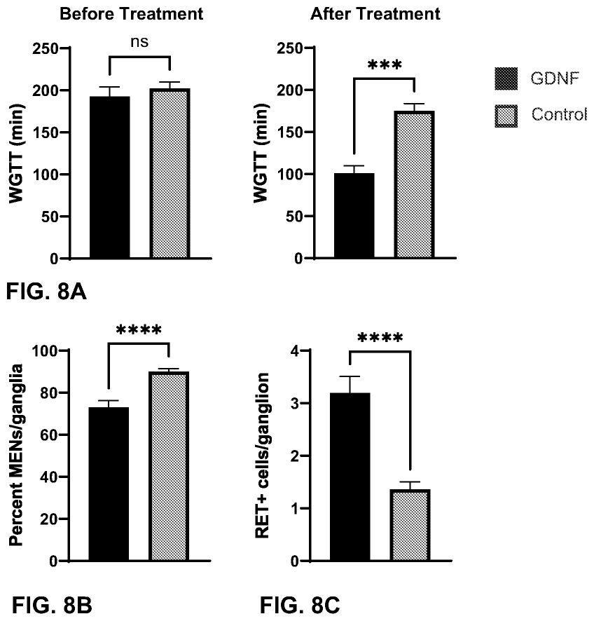

GDNF reverts aging in the ENS to normalize intestinal motility. The inventors have previously shown that aging is associated with retardation of intestinal motility^{58,59}. It was hypothesized that this may be a consequence of the replacement of the juvenile NENs population by MENs and therefore GDNF supplementation, by restoring a more balanced MENs:NENs ratio, may prevent age related changes in motility. 17-month-old male mice (at an age where NENs constitute only ˜5% of all myenteric neurons; FIG. 6C) were studied before and after they received 10 days of intraperitoneal injection of GDNF or saline (n=5 for each cohort). While the two cohorts showed no significant difference in their intestinal transit times at baseline (WGTT (in min) Control: 175.0±11.55 SEM; GDNF: 202.4±7.60 SEM, p=0.50), GDNF treatment caused significant improvement in intestinal transit (WGTT (in min) Control: 192.8±8.89 SEM; GDNF: 101.0±8.91 SEM, p=0.0004), reaching levels previously observed in healthy mice (FIGS. 8A; 7D). GDNF treatment did not change the average neuronal numbers in the ileal myenteric ganglia (n=50 ganglia/cohort; Control: 18.18±1.54 SEM, GDNF: 16.76±1.25 SEM, p=0.47), but significantly reduced the proportions of MHCst⁺ MENs (n=50 ganglia/cohort; Control: 90.03 f 1.44 SEM, GDNF: 72.99±3.24 SEM, p<0.0001) while increasing the numbers of RET NENs (Control: n=42 ganglia, 1.35±0.14 SEM, GDNF: n=41 ganglia 3.19±0.31 SEM, p<0.0001) (FIGS. 8B, 8C; 15A-15D).

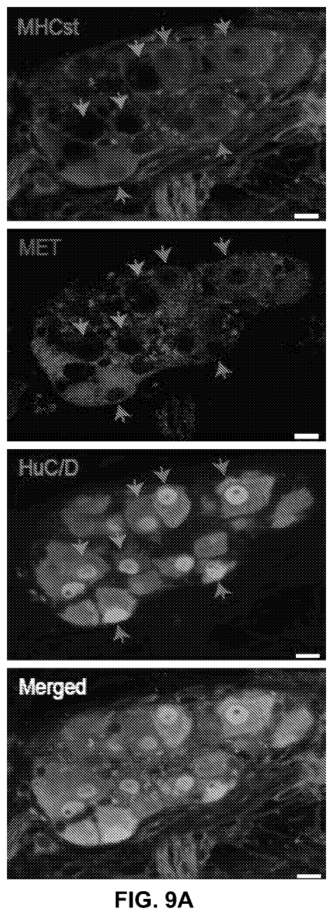

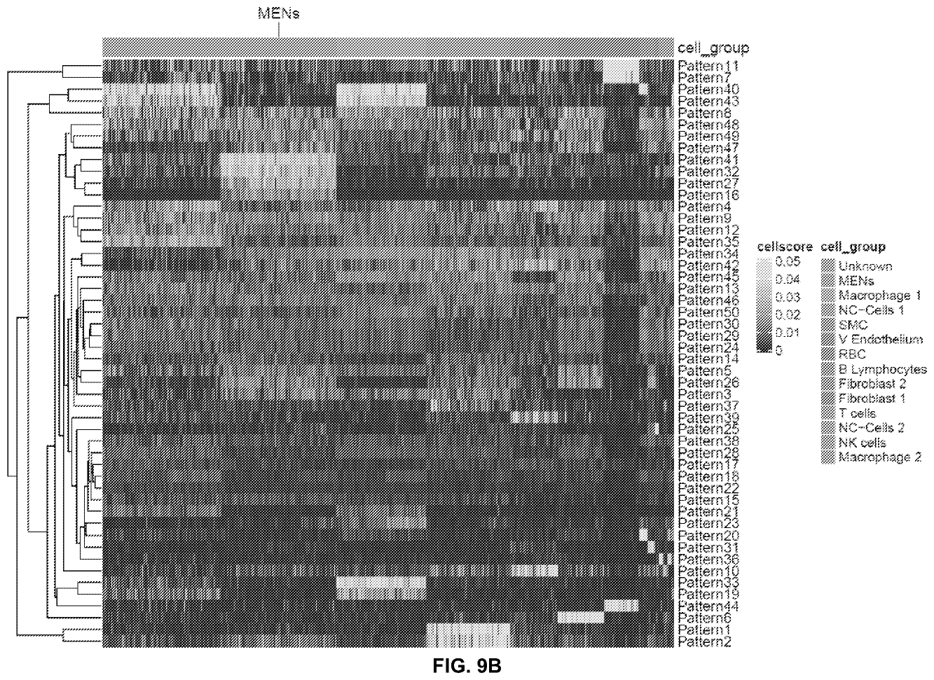

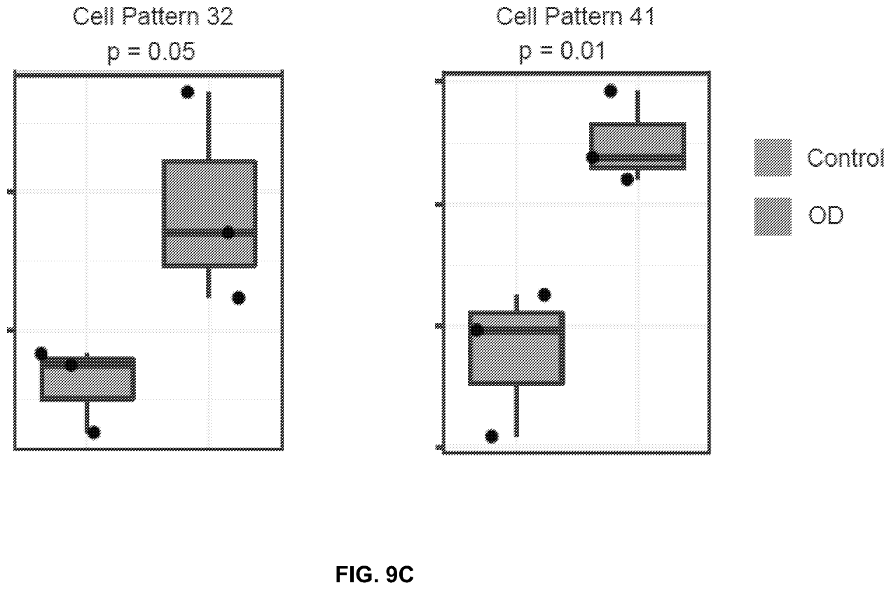

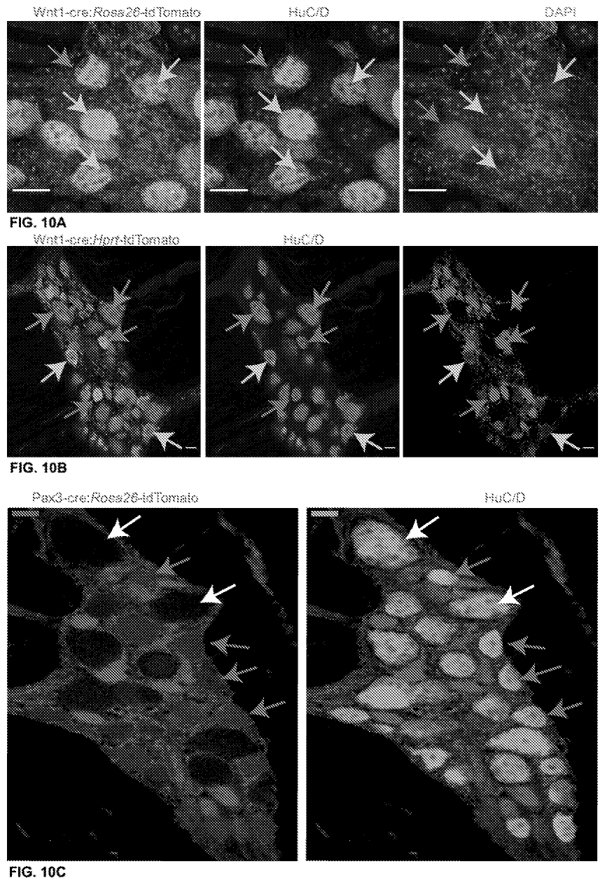

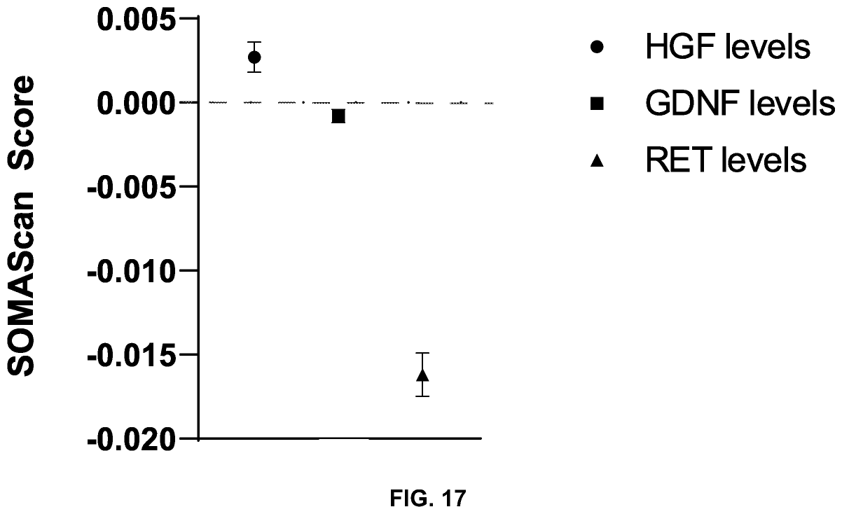

MENs in healthy and diseased human gut. To understand the relevance of the observations to human health and disease, we examined the expression of MENs markers MHCst and MET in LM-MP tissues from adult humans with no known gut motility disorder and found them to be expressed by a population of myenteric neurons, suggesting the presence of MENs in normal adult human ENS (FIG. 9A; 16A-16C). The plasma proteome levels were queried from the LonGenity cohort of 1,025 aging individuals and found that HGF levels correlated positively, while GDNF and RET levels correlated negatively with age (FIG. 17)⁶⁰, evidencing parallels between the data herein (FIGS. 6A-6G) from murine intestine and human plasma proteome data.

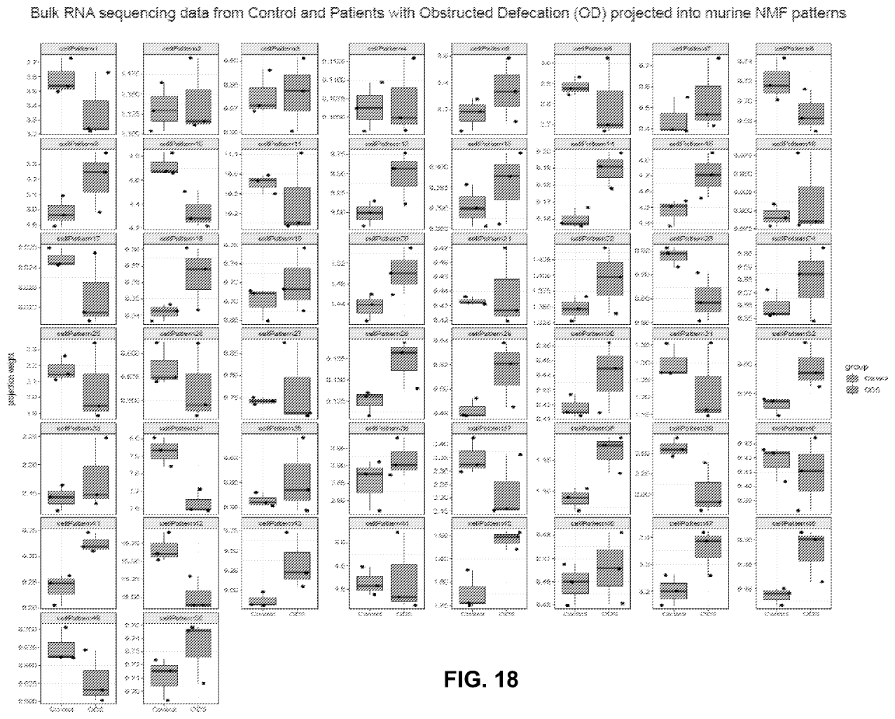

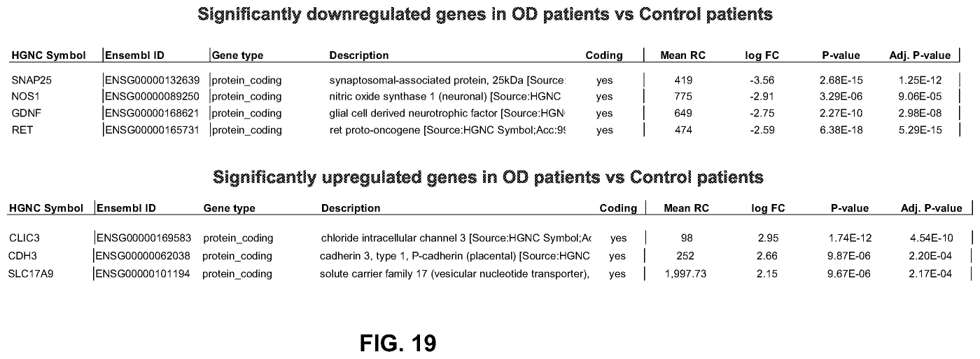

Next, it was tested whether gut dysfunction associated with pathological reductions in NENs-signaling mechanisms are also associated with increased abundance of MENs. For this, previously generated and publicly available transcriptomic data was mined from control tissues from humans with normal gut motility and from patients with obstructed defecation (OD), which is a chronic intestinal dysmotility disorder⁵⁷. In these datasets, Kim et al have previously shown that OD patients have significantly reduced intestinal expression of Gdnf and Ret⁵⁷. It was tested whether the reduced expression of NENs-regulatory genes Gdnf and Ret in OD patients is associated with an increase in MENs-specific transcriptional signature. The inventors have previously published a bioinformatic approach, which allows for learning latent space representations of gene expression patterns using single cell transcriptomic data, which include patterns that correspond to cell-type-specific gene expression signatures⁶¹. It can then project new single cell and bulk RNA transcriptomic datasets into these learned patterns to accurately quantify the differential use of these cell-type-specific signatures in transcriptomic data across platforms, tissues, and species⁶¹. In this instance, gene expression patterns were learned using non-negative matrix factorization (NMF) dimensionality reduction on the murine single cell transcriptomic data. In this manner, the scRNAseq data was decomposed into 50 distinct NMF-patterns^{61,62} (FIG. 9B). From the reduced set of dimensions, 4-different MENs-specific transcriptional signature patterns were identified by correlation of pattern usage matrix with our cellular annotations (FIG. 9B). Next, using the transfer learning tool ProjectR^{61,63}, which allowed for the query of bulk-RNAseq data across species to estimate relative use of MENs-specific NMF-patterns, were tested whether the contribution of MENs-specific NMF-patterns was significantly altered between transcriptomes of OD patients and healthy humans (n=3/group)⁵⁷. This projection indicated that 2 of the 4 MENs-specific NMF-patterns were significantly upregulated in OD samples compared to controls (Student's t-test, p<0.05) (FIGS. 9C, 18), providing evidence that intestinal dysmotility in OD patients is associated with a relative increase in MENs-specific transcriptional signatures. The differential gene analyses performed by Kim et al was further queried and found that important NEN genes Snap25, Gdnf, Ret, Nos1 were significantly downregulated while the MEN marker genes identified in this study (Clic3, Cdh3, Slc17a9) (FIG. 19) were significantly upregulated. The presence of human myenteric neurons expressing MENs markers, along with the altered NENs and MENs-signaling in aging humans, and the relative increase in MENs-specific transcriptomic signatures in patients with gut dysmotility provides evidence that the lineage dynamics identified in murine ENS are also of importance in the human ENS.

Discussion

Current dogma states that the adult ENS is exclusively derived from neural crest precursors that populate the gut in early embryonic life⁶⁴. The results of this study indicate a much more complex system, one in which the fetal and early life “juvenile” ENS consisting of neural crest-derived enteric neurons (NENs) is incrementally replaced during maturation by mesoderm-derived enteric neurons (MENs); eventually, the aging ENS consists almost exclusively of the neurons of the MEN lineage. This study also provides the first definitive evidence of a significant mesodermal contribution to any neural tissue. Previously, the mesoderm and especially the Lateral Plate Mesoderm (LPM) was known to give rise to diverse cell-types within the viscera, including several in the gut⁶⁵, and the study here shows that cells of this embryonic derivation also give rise to ENS neurons. A previous report on a dual origin of the ENS described adult enteric neurons from Foxa2⁺ or Sox17⁺ precursors¹⁴ and inferred that these were endodermal in origin. However, Foxa2 and Sox17 are also expressed by mesoderm-derived cells²²⁻²⁵. By contrast, using two NC-lineage-traced mouse lines (Wnt1-cre and Pax3-cre), two lineage-traced mouse lines marking mesodermal derivatives (Tek-cre, and Mesp1-cre), and robust adult mesoderm markers, a population of Mesp1-derived MHCst- and MET-expressing adult enteric neurons was identified, which makes up the entire non-NC-derived population of myenteric neurons. These results provide evidence that the second source of adult enteric neurons is the mesoderm, and not the endoderm. Importantly, it was confirmed herein, of MHCst and MET expression in many enteric neurons in adult humans, providing evidence that the mesoderm-derived ENS may be a feature common to mice and humans alike. MENs in both species can readily be identified by their expression of MHCst and MET, thus providing a convenient tool to further discriminate and study these neurons.

In the single cell transcriptomic analysis, the markers Calcb, Met, and Cdh3 were used that were validated to identify and annotate the MEN cell cluster. When compared with the Ret and Sox10-expressing NC-derived cell cluster, an additional set of marker genes were found to be expressed widely or selectively within MENs. Some of these, such as Slpi, Aebp1, Clic3, and Fmo2 have not been previously described in enteric neurons, while others (Ntf3, Il18, and Cftr) are known to be expressed^{2,47,66}. The differential expression of genes between the two neuronal populations (MENs versus NENs) provides evidence of putative specialized functional roles of these subpopulations when they co-exist in adults. Some of the prior scRNAseq analysis of post-natal ENS did not detect and characterize the true identity of the MEN lineage. This is because previous studies were performed either exclusively on neural crest-derived ENS cells⁴⁵, or when done in a more agnostic manner, only applied known canonical neural markers to identify the ENS population^{46,67}. Another recent study on scRNAseq of enteric neurons detected several neurons with a ‘mesenchymal’ transcriptomic signature⁶⁸. In this study, May-Zhang et al.⁶⁸ show that these ‘mesenchymal’ neurons exclusively express Ntf3 and Cdh3 which are detected as highly expressed in our MENs scRNAseq dataset. However, May-Zhang-et al did not further interrogate the developmental origins of the ‘mesenchymal’ enteric neurons to establish the etiology of their distinct transcriptomic signature. Thus, the discovery of the distinct identity and germ-layer derivation of MENs would not have been possible.

Since the scRNAseq data herein highlighted the lineage-specific nature of the expression of Ret and Met, it was studied whether these genes regulated the origin, expansion, and maintenance of the neuronal populations that express them. While it is known that Gdnf expression in the mature gut is significantly downregulated^{41,49,54}, the functional consequences of this loss have been unclear¹⁹. It was found that reduced GDNF-RET signaling drives the age-dependent loss of NENs as this loss can be slowed or reversed by GDNF supplementation and accelerated by further reduction of Ret expression. In aging animals, GDNF-driven resuscitation of NENs was associated with a functional normalization of age-associated delay in intestinal transit. These results identify a novel role for GDNF in maintaining or resuscitating the canonical NEN population, while preserving overall enteric neuronal numbers. In the last few years, studies have focused on identifying juvenile protective factors (JPFs), the loss of which correlates with or drives maturation- and aging-associated phenotypes⁶⁹. In this context, GDNF may therefore qualify as a JPF or a senolytic as its presence maintains the dominance of NENs in maturing ENS, corresponding to a juvenile phenotype; and its re-introduction promotes and resuscitates the genesis of NENs in adult and aging gut to restore healthy gut function. The exact nature of the cells that respond to GDNF re-introduction and generate NENs is yet unknown, but it can be hypothesized that these may include Nestin⁺ enteric neural stem cells and/or GDNF-responsive Schwann cells^{70,71}.

The observed mutually exclusive expression of Ret and Met by the two lineages of enteric neurons in the mature adult ENS is consistent with an earlier study that reported that a deletion of Met in the Wnt1-cre expressing NC-lineage did not alter the abundance of MET⁺ neurons in the adult ENS⁴¹. In this study, it was also shown that MENs are dependent on HGF-MET signaling in a manner analogous to the requirement of GDNF-RET signaling by NENs. Further, Ret haploinsufficiency-mediated loss of the NEN lineage causes a proportional increase in the MEN lineage. This provides evidence of the presence of yet unknown quorum sensing signaling mechanisms between the two lineages that reciprocally regulate their populations to maintain the structure of the post-natal ENS. This also implies the existence of a precursor cell responsible for the expansion of the MEN population in a manner analogous to what we have previously described for NENs⁷¹.

The validation of mesodermal markers (MHCst and MET) in a subset of adult human ENS neurons suggests that our findings may be of clinical importance. The loss of NENs and the corresponding dominance of MENs begins in early adulthood and maybe viewed as part of the normal maturation of the ENS. However, because of its progressive nature, it may have pathological implications for the elderly gut. Many gastrointestinal motility functions are compromised with advancing age, resulting in clinically significant disorders⁷². A progressive imbalance in these two populations in favor of MENs with age may therefore have functional consequences. Although the exact mechanisms will need to be worked out, the results herein indicate that a MENs-predominant ENS is associated with significant differences in gut motility. Understanding the forces that regulate parity between these two different sources of neurogenesis therefore holds the promise of arresting or reverting progressive loss of gut motility with increasing age. The results herein also have implications for the pathogenesis of disordered motility unrelated to aging, as downregulation of Gdnf and Ret expression has been associated with diverticular disease and obstructed defecation in adult patients^{57,73,74}. GWAS analyses further showed that Gdnf gene was in the eQTL associated with increased incidence of diverticular disease⁷⁵. It is in this context that the identification of SNAP-25 as a NENs marker gene and the transcriptomic pattern analyses of patients with chronic obstructed defecation are significant. Expression of Snap25 is upregulated by GDNF and is significantly downregulated in gut tissues of patients with diseases associated with significant reduction in GDNF-RET signaling (diverticular disease and obstructed defecation)^{48,57,73}. Thus, while earlier thought to be a pan-neuronal marker^{46,76}, establishing the identity of SNAP-25 as a NEN lineage-restricted marker provides us not only with an important tool to query proportions of NENs in murine and human tissue but also suggests the NENs-limited nature of prior observations based on Snap25 transgenic animals^{76,77}. Gut dysmotility disorders that present with conserved ENS neuronal numbers⁷³ have puzzled investigators on the etiology of the disease. The results herein suggest an explanation based on NENs-to-MENs lineage shifts that conserve overall enteric neuronal numbers with variable effects on function.

In conclusion, the ENS of the juvenile and mature mammalian gut is dominated by two different classes of neurons that have distinct ontogenies and trophic factors, associated with functional differences. The shift in neuronal lineage may represent a functional adaptation to changes in nutrient handling, microbiota or other physiological factors associated with different ages. Further research in the regulation of the parity between these two nervous systems in humans during the lifetime will be important to advance our understanding of the adult ENS and the treatment of age-related and other pathological disorders of gut motility.

TABLE 2
Age-wise distribution of Wnt1-cre:tdTomato
negative neurons/ganglia

| P11 | P22 | P60 | P180 | P510 |
|---|---|---|---|---|
| 1.2 | 45.45454 | 23.33333 | 93.93939 | 95.65217 |
| 0 | 42.85714 | 14.28571 | 100 | 100 |
| 0 | 50 | 10 | 100 | 97.53086 |
| 6 | 66.66666 | 28.20513 | 100 | 100 |
| 0 | 21.05263 | 25 | 100 | 89.28571 |
| 0 | 41.66667 | 0 | 0 | 100 |
| 0 | 20 | 0 | 0 | 100 |
| 0 | 35 | 60.60606 | 33.33333 | 100 |
| 0 | 36.84211 | 75 | 95 | 100 |
| 14.2 | 44.44444 | 91.66666 | 80 | 95.65217 |
| 10 | 45.45454 | 100 | 73.33334 | 100 |
| 87.5 | 5.555555 | 33.33333 | 75 | 100 |
| 0 | 7.142857 | 40 | 87.5 | 100 |
| 0 | 20 | 33.33333 | 87.5 | 100 |
| 25 | 0 | 46.34146 | 90.90909 | 100 |
| 0 | 25 | 57.14286 | 88.88889 | 100 |
| 0 | 50 | 44.44444 | 80 | 100 |
| 6 | 53.33333 | 60 | 20 | 100 |
| 0 | 8.333333 | 40 | 100 | 100 |
| 1.5 | 20 | 38.09524 | 100 | 89.74359 |
| 0 | 0 | 58 | 94.28571 | 100 |
| 0 | 14.28571 | 36.36364 | 0 | 100 |
| 0 | 11.76471 | 13.63636 | 14 | 100 |
| 0 | 25 | 46.66667 | 44.44444 | 95.83333 |
| 0 | 42.85714 | 52.38095 | 91.66666 | 90 |
| 0 | 40 | 62.5 | 80 | 100 |
| 0 | 23.52941 | 68.42105 | 53.33333 | 66.66667 |
| 0 | 60 | 38.88889 | 92.30769 | 100 |
| 0 | 22.22222 | 62.16216 | 88.88889 | 100 |
| 1 | 8.333333 | 50 | 76.66666 | 100 |
| 0 | 30.76923 | 85.71429 | 84.61539 | 100 |
| 0 | 12.5 | 52.63158 | 97.14286 | 92.15686 |
| 0 | 14.28571 | 50 | 100 | 97.4359 |
| 2 | 9.523809 | 56 | 47.82609 | 100 |
| | 32.25806 | 57.14286 | 14.70588 | 100 |
| | 36.36364 | 45.45454 | 25 | 66.66667 |
| | 50 | 42.85714 | 93.75 | 77.77778 |
| | 63.63636 | | 65.21739 | 100 |
| | 29.16667 | | 7.692307 | 94.73684 |
| | 68 | | 0 | 86.66667 |
| | 29.16667 | | 63.63636 | 57.89474 |
| | 48 | | 58.82353 | 100 |
| | 43.75 | | 100 | 100 |
| | 40 | | 93.75 | 100 |
| | 0 | | 90.14085 | 81.81818 |
| | | | 94.44444 | 95 |
| | | | 76.66666 | 100 |
| | | | 75 | 100 |
| | | | 100 | 88.88889 |
| | | | 25 | 100 |
| | | | 97.56097 | 100 |
| | | | 100 | 100 |
| | | | 100 | 90.90909 |
| | | | 84.21053 | 100 |
| | | | 66.66666 | 100 |
| | | | 100 | 100 |
| | | | 40 | 100 |
| | | | 86.36364 | 100 |
| | | | 91.66666 | 100 |
| | | | 100 | 100 |
| | | | 84.375 | 95.2381 |
| | | | 91.30434 | 95.65217 |
| | | | 90.90909 | 100 |
| | | | 100 | 100 |
| | | | 100 | 100 |
| | | | 83.33334 | 93.33333 |
| | | | 50 | 90.90909 |
| | | | 96.15385 | |
| | | | 90.90909 | |

TABLE 3
Percentage of CGRP expressing neurons
in 30 40X fields imaged from 3 mice

| % neurons that are | % neurons that are |
|---|---|
| CGRP+ tdTomato+ | CGRP+ tdTomato− |
| 0.000 | 0.000 |
| 0.000 | 12.500 |
| 0.000 | 100.000 |
| 0.000 | 0.000 |
| 7.692 | 0.000 |
| 0.000 | 14.286 |
| 6.667 | 6.667 |
| 0.000 | 20.000 |
| 0.000 | 0.000 |
| 0.000 | 18.750 |
| 0.000 | 0.000 |
| 0.000 | 0.000 |
| 0.000 | 7.692 |
| 3.333 | 30.000 |
| 9.091 | 0.000 |
| 0.000 | 9.091 |
| 0.000 | 50.000 |
| 0.000 | 0.000 |
| 5.882 | 0.000 |
| 0.000 | 0.000 |
| 12.500 | 7.143 |
| 0.000 | 25.000 |
| 0.000 | 5.000 |
| 0.000 | 11.111 |
| 0.000 | 14.286 |
| 0.000 | 8.333 |
| 6.250 | 0.000 |
| 0.000 | 0.000 |
| 0.000 | 31.818 |
| 0.000 | 25.000 |
| 15.385 | 14.286 |
| 0.000 | 21.429 |
| 0.000 | 0.000 |
| 0.000 | 20.000 |

TABLE 4
Percentage of NOS1⁺ neurons of the two different
lineages in 32 ganglia imaged from 3 mice

| % NOS1+ neurons that are | % NOS1+ neurons that are |
|---|---|
| Wnt1-cre:tdTomato+ | Wnt1-cre:tdTomato− |
| 55.55556 | 44.44444 |
| 40 | 60 |
| 75 | 25 |
| 27.77778 | 72.22222 |
| 76.92308 | 23.07692 |
| 20 | 80 |
| 66.66667 | 33.33333 |
| 60 | 40 |
| 66.66667 | 33.33333 |
| 54.54545 | 45.45455 |
| 81.81818 | 18.18182 |
| 71.42857 | 28.57143 |
| 58.33333 | 41.66667 |
| 83.33333 | 16.66667 |
| 80.95238 | 19.04762 |
| 66.66667 | 33.33333 |
| 55.55556 | 44.44444 |
| 72.72727 | 27.27273 |
| 75 | 25 |
| 63.63636 | 36.36364 |
| 66.66667 | 33.33333 |
| 42.85714 | 57.14286 |
| 37.5 | 62.5 |
| 57.14286 | 42.85714 |
| 50 | 50 |
| 80 | 20 |
| 90.90909 | 9.090909 |
| 36.36364 | 63.63636 |
| 87.5 | 12.5 |
| 77.77778 | 22.22222 |
| 100 | 0 |
| 50 | 50 |

TABLE 5
Percentage of Mesp1-cre:tdTomato+
neurons in 31 ganglia imaged from 3 mice

| Mesp1-cre:tdTomato− neurons | Mesp1-cre:tdTomato+ neurons |
|---|---|
| 100 | 0 |
| 97 | 3 |
| 91 | 9 |
| 100 | 0 |
| 44 | 56 |
| 0 | 100 |
| 55 | 45 |
| 40 | 60 |
| 40 | 60 |
| 0 | 100 |
| 25 | 75 |
| 56 | 44 |
| 45 | 55 |
| 39 | 61 |
| 38 | 63 |
| 38 | 62 |
| 71 | 29 |
| 73 | 27 |
| 41 | 59 |
| 30 | 70 |
| 0 | 100 |
| 87 | 13 |
| 91 | 9 |
| 97 | 3 |
| 21 | 79 |
| 48 | 52 |
| 38 | 62 |
| 43 | 57 |
| 50 | 50 |
| 38 | 62 |
| 59 | 41 |

TABLE 6
Numbers of HuC/D+ neurons/ganglia in Control
vs GDNF infused groups in neonatal (P10 mice)

| Saline Control | GDNF |
|---|---|
| 17 | 27 |
| 23 | 38 |
| 7 | 13 |
| 3 | 34 |
| 12 | 6 |
| 5 | 24 |
| 19 | 17 |
| 36 | 16 |
| 23 | 21 |
| 33 | 33 |
| 12 | 18 |
| 43 | 6 |
| 8 | 12 |
| 110 | 54 |
| 16 | 45 |
| 39 | 18 |
| 25 | 6 |
| 20 | 11 |
| 17 | 4 |
| 6 | 4 |
| 3 | 11 |
| 9 | 7 |
| 11 | 8 |
| 11 | 12 |
| 10 | 11 |
| 16 | 8 |
| 5 | 6 |
| 26 | 13 |
| 7 | 8 |
| 4 | 25 |
| 9 | 26 |
| 29 | 3 |
| 3 | 5 |
| 9 | 24 |
| 10 | 30 |
| 14 | 16 |
| 4 | 5 |
| 31 | 8 |
| 24 | 3 |
| 17 | 24 |
| 11 | 22 |
| 22 | 45 |
| 7 | 4 |
| 7 | 37 |
| 14 | 26 |
| 10 | 28 |
| 14 | 78 |
| 15 | 6 |
| 31 | 58 |
| 25 | 39 |
| 33 | 44 |
| 54 | 32 |
| 25 | 42 |
| 5 | 41 |
| 17 | 27 |
| 61 | 35 |
| 31 | 29 |
| 32 | 48 |
| 44 | |
| 4 | |
| 25 | |
| 31 | |
| 39 | |
| 25 | |
| 35 | |

TABLE 7
% Wnt1-cre:tdTomato− MENs/ganglia in Control
vs GDNF infused groups in neonatal (P10 mice)

| Saline Control | GDNF |
|---|---|
| 0 | 0 |
| 0 | 3 |
| 43 | 0 |
| 67 | 12 |
| 33 | 0 |
| 0 | 0 |
| 16 | 0 |
| 14 | 19 |
| 74 | 0 |
| 3 | 6 |
| 0 | 0 |
| 2 | 0 |
| 0 | 0 |
| 0 | 0 |
| 0 | 0 |
| 3 | 0 |
| 0 | 17 |
| 0 | 0 |
| 0 | 25 |
| 17 | 75 |
| 0 | 9 |
| 0 | 0 |
| 0 | 0 |
| 0 | 0 |
| 0 | 18 |
| 0 | 63 |
| 40 | 0 |
| 62 | 0 |
| 14 | 0 |
| 25 | 0 |
| 22 | 0 |
| 0 | 0 |
| 0 | 0 |
| 0 | 0 |
| 0 | 0 |
| 0 | 0 |
| 0 | 0 |
| 3 | 0 |
| 0 | 0 |
| 0 | 4 |
| 9 | 9 |
| 0 | 7 |
| 43 | 0 |
| 14 | 3 |
| 43 | 4 |
| 30 | 4 |
| 7 | 4 |
| 53 | 0 |
| 35 | 0 |
| 40 | 8 |
| 33 | 11 |
| 31 | 0 |
| 60 | 0 |
| 100 | 5 |
| 24 | 4 |
| 16 | 11 |
| 29 | 0 |
| 28 | 0 |
| 39 | |
| 75 | |
| 36 | |
| 16 | |
| 38 | |
| 44 | |
| 57 | |

TABLE 8
Numbers of HuC/D+ neurons/ganglia in
Control vs GDNF infused groups in adult mice

| Saline Control | GDNF |
|---|---|
| 18 | 6 |
| 20 | 54 |
| 33 | 32 |
| 47 | 52 |
| 49 | 41 |
| 24 | 19 |
| 38 | 33 |
| 28 | 55 |
| 7 | 14 |
| 46 | 41 |
| 29 | 28 |
| 19 | 41 |
| 22 | 4 |
| 15 | 20 |
| 28 | 56 |
| 47 | 5 |
| 9 | 63 |
| 49 | 22 |
| 16 | 27 |
| 39 | 20 |
| 7 | 51 |
| 39 | 49 |
| 17 | 31 |
| 11 | 39 |
| 43 | 7 |
| 50 | 20 |
| 11 | 37 |
| 34 | 18 |
| 29 | 12 |
| 12 | 21 |
| 14 | 23 |
| 3 | 20 |
| 12 | 32 |
| 58 | 25 |
| 50 | 55 |
| 43 | 42 |
| 13 | 5 |
| 66 | 18 |
| 10 | 14 |
| 18 | 14 |
| 16 | 60 |
| 42 | 63 |
| 8 | 49 |
| 28 | 46 |
| 10 | 37 |
| 33 | 13 |
| 48 | 57 |
| 13 | |
| 24 | |
| 4 | |
| 14 | |
| 57 | |
| 3 | |
| 33 | |
| 15 | |
| 22 | |
| 16 | |

TABLE 9
Percent Wnt1-cre:tdTomato− MENs/ganglia
in Control vs GDNF infused groups in adult mice

| Saline Control | GDNF |
|---|---|
| 64 | 70 |
| 0 | 63 |
| 22 | 61 |
| 44 | 60 |
| 7 | 60 |
| 45 | 65 |
| 33 | 61 |
| 66 | 59 |
| 70 | 49 |
| 55 | 68 |
| 56 | 41 |
| 54 | 43 |
| 2 | 45 |
| 57 | 65 |
| 31 | 63 |
| 55 | 50 |
| 59 | 62 |
| 54 | 61 |
| 26 | 48 |
| 62 | 50 |
| 56 | 66 |
| 62 | 64 |
| 46 | 60 |
| 52 | 55 |
| 61 | 70 |
| 52 | 65 |
| 70 | 59 |
| 55 | 64 |
| 63 | 45 |
| 70 | 51 |
| 49 | 57 |
| 37 | 50 |
| 53 | 67 |
| 58 | 58 |
| 58 | 59 |
| 49 | 56 |
| 70 | 50 |
| 62 | 53 |
| 50 | 49 |
| 48 | 41 |
| 51 | 58 |
| 58 | 53 |
| 45 | 52 |
| 52 | 50 |
| 60 | 65 |
| 55 | 62 |
| 53 | 61 |
| 39 | |
| 58 | |
| 45 | |
| 56 | |
| 56 | |
| 37 | |
| 64 | |
| 57 | |
| 56 | |
| 64 | |

TABLE 10
% Wnt1-cre:tdTomato− MENs/ganglia in Control
vs HGF infused groups in neonatal (P10 mice)

| Saline Control | HGF |
|---|---|
| 15 | 10 |
| 25 | 10 |
| 10 | 22 |
| 10 | 17 |
| 18 | 15 |
| 27 | 14 |
| 6 | 6 |
| 29 | 5 |
| 13 | 6 |
| 15 | 21 |
| 18 | 9 |
| 15 | 6 |
| 46 | 27 |
| 48 | 14 |
| 9 | 12 |
| 14 | 28 |
| 21 | 36 |
| 7 | 18 |
| 8 | 5 |
| 14 | 24 |
| 26 | 20 |
| 11 | 41 |
| 15 | 8 |
| 9 | 6 |
| 7 | 10 |
| 32 | 19 |
| 23 | 26 |
| 30 | 20 |
| 30 | 18 |
| 18 | 11 |
| 7 | 22 |
| 23 | 13 |
| 57 | 51 |
| 8 | 15 |
| 12 | 18 |
| 22 | 17 |
| 4 | 25 |
| 39 | 63 |
| 30 | 11 |
| 12 | 15 |
| 10 | 26 |
| 10 | 4 |
| 12 | 17 |
| 20 | 12 |
| 10 | 36 |
| 26 | 11 |
| 13 | 6 |
| 9 | 12 |
| 12 | 23 |
| 20 | 55 |
| 15 | 8 |
| 34 | 10 |
| 58 | 25 |
| 19 | 19 |
| 8 | 16 |
| 6 | 38 |
| 11 | 11 |
| 30 | 63 |
| 6 | 8 |
| 30 | 19 |
| 5 | 7 |
| 22 | 28 |
| 20 | 30 |
| 7 | 31 |
| 18 | 6 |
| 13 | 36 |
| 24 | 23 |
| 28 | 18 |
| 28 | 20 |
| 14 | 14 |
| 14 | 4 |
| 10 | 12 |
| 5 | 4 |
| 24 | 10 |
| 6 | 30 |
| 5 | 45 |
| 15 | 4 |
| 6 | 7 |
| 19 | 9 |
| 21 | 11 |
| 18 | 9 |
| 30 | 17 |
| 10 | 12 |
| 11 | 14 |
| 18 | 41 |
| 14 | 37 |
| 17 | 22 |
| 10 | 46 |
| 19 | 27 |
| 27 | 26 |
| 10 | 57 |
| 22 | 40 |
| 25 | 28 |
| 27 | 13 |

TABLE 11
Percent Wnt1-cre:tdTomato− MENs/ganglia
in Control vs HGF infused groups in P10 mice

| Saline Control | HGF |
|---|---|
| 20 | 40 |
| 20 | 72 |
| 14 | 50 |
| 41 | 80 |
| 27 | 22 |
| 14 | 4 |
| 50 | 33 |
| 60 | 55 |
| 33 | 54 |
| 67 | 73 |
| 44 | 28 |
| 17 | 33 |
| 7 | 20 |
| 14 | 19 |
| 0 | 44 |
| 50 | 71 |
| 6 | 57 |
| 39 | 43 |
| 20 | 63 |
| 8 | 71 |
| 0 | 42 |
| 0 | 36 |
| 13 | 27 |
| 17 | 22 |
| 10 | 0 |
| 11 | 53 |
| 31 | 70 |
| 0 | 50 |
| 17 | 27 |
| 9 | 28 |
| 18 | 29 |
| 46 | 30 |
| 16 | 60 |
| 27 | 0 |
| 11 | 75 |
| 35 | 50 |
| 32 | 0 |
| 11 | 26 |
| 18 | 27 |
| 27 | 58 |
| 31 | 30 |
| 50 | 50 |
| 12 | 50 |
| 42 | 30 |
| 8 | 50 |
| 18 | 31 |
| 83 | 8 |
| 33 | 11 |
| 9 | 42 |
| 44 | 60 |
| 100 | 60 |
| 90 | 35 |
| 44 | 22 |
| 58 | 32 |
| 31 | 50 |
| 24 | 17 |
| 9 | 82 |
| 8 | 80 |
| 100 | 100 |
| 11 | 53 |
| 100 | 40 |
| 86 | 23 |
| 57 | 5 |
| 71 | 43 |
| 83 | 61 |
| 42 | 15 |
| 70 | 46 |
| 44 | 54 |
| 70 | 61 |
| 64 | 79 |
| 75 | 79 |
| 33 | 90 |
| 75 | 80 |
| 60 | 67 |
| 33 | 100 |
| 24 | 80 |
| 50 | 87 |
| 57 | 100 |
| 44 | 74 |
| 27 | 76 |
| 22 | 50 |
| 12 | 60 |
| 25 | 50 |
| 14 | 45 |
| 15 | 78 |
| 14 | 64 |
| 18 | 76 |
| 13 | 60 |
| 22 | 63 |
| 23 | 63 |
| 11 | 90 |
| 5 | 77 |
| 4 | 84 |
| 0 | 67 |
| 38 | 0 |
| 14 | 0 |
| 22 | 0 |
| 57 | 0 |
| 18 | 0 |

TABLE 12

| | P10 age | P30 age | P90 age |
|---|---|---|---|
| | Western Blot Data for GDNF | | |
| | Data is represented as FL intensity | | |
| | of GDNF over FL intensity of Beta Actin | | |
| Mouse1 | 1.372414 | 0.069322 | 0.069403 |
| Mouse2 | 1.271605 | 0.095342 | 0.031436 |
| Mouse3 | 1.392405 | 0.052809 | 0.053185 |
| | Western Blot Data for HGF | | |
| | Data is represented as FL intensity | | |
| | of HGF over FL intensity of Beta Actin | | |
| Mouse1 | 0.008586 | 0.007423 | 0.02256 |
| Mouse2 | 0.006579 | 0.017765 | 0.01304 |
| Mouse3 | 0.006333 | 0.011445 | 0.016552 |

TABLE 13
Normalized HGF transcripts per age HGF Ct is
normalized to that of housekeeping gene Hprt

| | P10 age | P30 age | P90 age |
|---|---|---|---|
| Mouse 1 | 2.146509 | 5.442223 | 6.114431 |
| Mouse1 | 1.866098 | 4.747683 | 5.337063 |
| Mouse2 | 0.543238 | 2.919882 | 4.745646 |
| Mouse2 | 0.658921 | 2.683158 | 4.122825 |
| Mouse3 | 0.788646 | 2.703377 | 5.593155 |
| Mouse3 | 0.884356 | 2.928401 | 5.824324 |

TABLE 14
% MHCst + HuC/D + neurons/ganglia in the Ret heterozygous
and Control mice at two different ages

| 9 week Ret | 9 week | 16 week Ret | 16 week |
|---|---|---|---|
| heterozygous | Control | heterozygous | Control |
| mutant | Wildtype | mutant | Wildtype |
| 33 | 29 | 100 | 63 |
| 29 | 78 | 95 | 42 |
| 52 | 60 | 77 | 48 |
| 58 | 57 | 57 | 38 |
| 100 | 29 | 100 | 66 |
| 50 | 58 | 100 | 85 |
| 60 | 41 | 100 | 44 |
| 50 | 19 | 84 | 54 |
| 74 | 45 | 100 | 47 |
| 55 | 30 | 100 | 20 |
| 11 | 31 | 100 | 75 |
| 76 | 68 | 80 | 89 |
| 50 | 81 | 93 | 100 |
| 81 | 32 | 100 | 67 |
| 79 | 68 | 100 | 65 |
| 60 | 70 | 89 | 28 |
| 77 | 80 | 82 | 61 |
| 78 | 32 | 70 | 47 |
| 77 | 62 | 91 | 79 |
| 60 | 32 | 71 | 79 |
| 67 | 22 | 89 | 55 |
| 50 | 50 | 89 | 39 |
| 70 | 10 | 83 | 92 |
| 54 | 40 | 67 | 65 |
| 56 | 80 | 100 | 77 |
| 82 | 90 | 25 | 82 |
| 90 | 26 | 80 | 80 |
| 72 | 100 | 88 | 63 |
| 38 | 100 | 58 | 77 |
| 0 | 100 | 89 | 76 |
| 14 | 25 | 100 | 69 |
| 81 | 55 | 84 | 38 |
| 88 | 40 | 94 | 33 |
| 63 | 15 | 60 | 33 |
| 63 | 65 | 100 | 56 |
| 75 | 20 | 88 | 48 |
| 100 | 42 | 94 | 68 |
| 100 | | 100 | 71 |
| | | 100 | 86 |
| | | 100 | 60 |
| | | 75 | 55 |
| | | 75 | 80 |
| | | 70 | 53 |
| | | 100 | 21 |
| | | 68 | 74 |
| | | 100 | 76 |
| | | 78 | 44 |
| | | 26 | 56 |
| | | 57 | 50 |
| | | 35 | 33 |
| | | 100 | 65 |
| | | 100 | 47 |
| | | 88 | 56 |
| | | 80 | 73 |
| | | 100 | 25 |
| | | | 50 |
| | | | 80 |
| | | | 50 |
| | | | 80 |
| | | | 14 |
| | | | 11 |
| | | | 29 |
| | | | 0 |
| | | | 100 |
| | | | 60 |

TABLE 15
Feret diameter (in um) of Wnt1-cre:tdTomato+
NENs and Wnt1-cre:tdTomato− MENs cells

| NENs | MENS |
|---|---|
| 7.68 | 24.15 |
| 8.05 | 22.07 |
| 11.63 | 17.6 |
| 13.14 | 20.78 |
| 9.34 | 18.61 |
| 15.98 | 15.58 |
| 10.32 | 13.44 |
| 11.74 | 13.96 |
| 10.44 | 16.34 |
| 10.24 | 11.34 |
| 13.1 | 13.16 |
| 12.8 | 14.74 |
| 8.44 | 13.12 |
| 11.72 | 13.77 |
| 12.15 | 19.67 |
| 14.68 | 24.39 |
| 11.74 | 18.87 |
| 15.1 | 18.02 |
| 13.44 | 20.95 |
| 13.18 | 22.21 |
| 12.69 | 15.56 |
| 13.26 | 20.95 |
| 9.34 | 13.87 |
| 13.87 | 16.12 |
| 13.52 | 14.2 |
| 10.3 | 14.42 |
| 10.68 | 14.58 |
| 11.93 | 16.81 |
| 15.51 | 20.9 |
| 12.14 | 13.68 |
| 10.92 | 20.99 |
| 15.73 | 14.86 |
| 17.92 | 13.6 |
| 12.53 | 19.37 |
| 15.98 | 13.29 |
| 13.6 | 14.84 |
| 11.48 | 15.41 |
| 10.98 | 14.6 |
| 16.6 | 12.61 |
| 10.87 | 18.87 |
| 15.35 | 21.67 |
| 11.43 | 23.88 |
| 12.93 | 16.53 |
| 13.04 | 26.93 |
| 9.61 | 19.56 |
| 9.79 | 18.87 |
| 9.9 | 18.49 |
| 12.17 | 15.51 |
| 9.46 | 25.69 |
| 13.5 | 17.34 |
| 15.83 | 21.18 |
| 8.28 | 17.58 |
| 13.94 | 26.58 |
| 9.41 | 28.4 |
| 12.76 | 17.89 |
| 12.22 | 31.64 |
| 17.19 | 15.96 |
| 16.05 | 14.28 |
| 10.85 | 19.77 |
| 13.1 | 16.28 |
| 12.18 | 17.15 |
| 12.28 | 15.02 |
| 14.39 | 14.25 |
| 17.92 | 19.79 |
| 16.73 | 15.66 |
| 12.28 | 23.39 |
| 13.43 | 25.82 |
| 12.84 | 21.49 |
| 13.59 | 14.26 |
| 15.89 | 14.53 |
| 13.05 | 15.4 |
| 9.6 | 11.58 |
| 10.5 | 15.39 |
| 15.31 | 12.87 |
| 12.47 | 13.55 |
| 18.24 | 15.06 |
| 14.78 | 13.41 |
| 22.28 | 27.92 |
| 13.86 | 28.26 |
| 14.25 | 22.84 |
| 13.62 | 24.93 |
| 13.8 | 19.25 |
| 12.6 | 21.68 |
| 13.34 | 18.99 |
| 13.3 | 16.75 |
| 17.71 | 18.13 |
| 9.66 | 17.85 |
| 11.49 | 14 |
| 11.88 | 16.63 |
| 12.58 | 13.56 |
| 9.85 | 12.09 |
| 21.68 | 11.1 |
| 13.22 | 13.8 |
| 11.72 | 19.25 |
| 9.37 | 18.93 |
| 13.06 | 11.04 |
| 13.39 | 13.84 |
| 13.75 | 16.93 |
| 13.68 | 17.28 |
| 9.07 | 13.25 |
| 19.8 | 20.1 |
| 18.62 | 25.45 |
| 17.29 | 18.93 |
| 15.16 | 13.03 |
| 9.27 | 13.01 |
| 12.12 | 13.8 |
| 11.6 | 17.11 |
| 13.14 | 13.16 |
| 14.99 | 13.01 |
| 15.07 | 19.18 |
| 12.13 | 22.4 |
| 9.32 | 14.94 |
| 9.17 | 17.42 |
| 15.24 | 18.36 |
| 11.04 | 19.77 |
| 9.79 | 14.06 |
| 12.19 | 17.63 |
| 13.31 | 16.16 |
| 10.34 | 11.85 |
| 9.74 | 10.53 |
| 16.73 | 11.57 |
| 17.08 | 17.63 |
| 9.21 | 15.67 |
| 10.98 | 14.19 |
| 18.82 | 14.97 |
| 16.26 | 14.98 |
| 19.97 | 12.64 |
| 17.79 | 16.87 |
| 17.52 | 21.49 |
| 12.3 | 13.12 |
| 16.71 | 17.2 |
| 11 | 26.79 |
| 12.21 | 19.7 |
| 14.35 | 19.06 |
| 15.17 | 18.69 |
| 13.58 | 24.6 |
| 11.6 | 21.36 |
| 11.82 | 23.55 |
| 14.74 | 11.74 |
| 11.63 | 14.01 |
| 11.51 | 18.63 |
| 9.8 | 14.01 |
| 9.94 | 12.02 |

TABLE 16
Percent Ret-CFP + HuC/D + neurons per ganglia with age

| 9 weeks | 16 weeks |
|---|---|
| 14 | 0 |
| 16 | 6 |
| 0 | 43 |
| 0 | 11 |
| 6 | 0 |
| 17 | 8 |
| 20 | 0 |
| 0 | 0 |
| 14 | 0 |
| 0 | 0 |
| 0 | 73 |
| 60 | 53 |
| 17 | 30 |
| 0 | 7 |
| 33 | 0 |
| 42 | 0 |
| 75 | 0 |
| 0 | 0 |
| 73 | 11 |
| 33 | 0 |
| 54 | 0 |
| 0 | 0 |
| 11 | 15 |
| 53 | 0 |
| 20 | 6 |
| 100 | 0 |
| 14 | 100 |
| 20 | 50 |
| 24 | 80 |
| 60 | 40 |
| 8 | 9 |
| 25 | 52 |
| 60 | 18 |
| 3 | 8 |
| 7 | 4 |
| 20 | 4 |
| 33 | 0 |

TABLE 17
Whole gut transit time in Wildtype and Ret
heterozygous mutant animals with age

| 9 | | 12 | | 14 | | 16 | |
|---|---|---|---|---|---|---|---|
| weeks | | weeks | | weeks | | weeks | |
| Wild | Ret | Wild | Ret | Wild | Ret | Wild | Ret |
| type | het | type | het | type | het | type | het |
| 131 | 143 | 83 | 71 | 107 | 106 | 111 | 157 |
| 119 | 91 | 89 | 98 | 103 | 93 | 130 | 122 |
| 59 | 93 | 88 | 106 | 120 | 190 | 106 | 194 |
| 135 | 105 | 100 | 124 | 141 | 189 | 116 | 237 |
| 113 | 73 | 106 | 89 | 111 | 100 | 130 | 200 |
| 88 | 134 | 86 | 128 | 117 | 190 | 122 | 102 |
| 103 | 87 | 110 | 77 | 166 | 107 | 116 | 185 |
| 86 | 83 | 78 | 73 | 110 | 207 | 140 | 101 |
| | 153 | | 82 | | 125 | | 119 |
| | 132 | | 124 | | 96 | | 156 |

TABLE 18
Whole gut transit time analyses of 17-month-old
aging mice before and after treatment with GDNF

| Before treatment | Before treatment | After treatment | After treatment | |
|---|---|---|---|---|
| Control group | GDNF group | Control group | GDNF group | |
| 210 min | 182 min | 210 min | 74 | min |
| 210 min | 210 min | 164 min | 110 | min |
| 210 min | 152 min | 168 min | 87 | min |
| 172 min | 210 min | 162 min | 123 | min |
| 210 min | 210 min | 171 min | 111 | min |

TABLE 19
Numbers of HuC/D-labeled neurons per myenteric ganglia in
Control and GDNF treatment groups in 17-month-old mice

| Control | GDNF |
|---|---|
| 16 | 34 |
| 8 | 4 |
| 3 | 7 |
| 20 | 19 |
| 28 | 4 |
| 25 | 20 |
| 12 | 16 |
| 16 | 3 |
| 13 | 4 |
| 29 | 26 |
| 10 | 14 |
| 28 | 28 |
| 32 | 33 |
| 4 | 11 |
| 12 | 31 |
| 6 | 19 |
| 11 | 17 |
| 23 | 14 |
| 45 | 10 |
| 38 | 12 |
| 18 | 9 |
| 13 | 28 |
| 13 | 17 |
| 32 | 17 |
| 30 | 27 |
| 9 | 21 |
| 42 | 35 |
| 24 | 13 |
| 19 | 4 |
| 8 | 9 |
| 17 | 19 |
| 25 | 10 |
| 3 | 25 |
| 17 | 21 |
| 25 | 15 |
| 26 | 25 |
| 22 | 3 |
| 17 | 14 |
| 3 | 8 |
| 16 | 16 |
| 34 | 12 |
| 25 | 34 |
| 16 | 18 |
| 3 | 8 |
| 6 | 6 |
| 8 | 22 |
| 33 | 17 |
| 20 | 14 |
| 3 | 21 |
| 3 | 24 |

TABLE 20
Percent MHCst neurons of all HuC/D-labeled neurons
in myenteric ganglia of GDNF-treated and saline-
treated control groups in 17-month-old mice.

| GDNF treated | Saline treated Control |
|---|---|
| 91.17647 | 87.5 |
| 100 | 100 |
| 100 | 100 |
| 94.73684 | 80 |
| 75 | 85.71429 |
| 80 | 92 |
| 56.25 | 91.66667 |
| 100 | 87.5 |
| 75 | 92.30769 |
| 65.38462 | 93.10345 |
| 35.71429 | 90 |
| 46.42857 | 82.14286 |
| 42.42424 | 93.75 |
| 81.81818 | 100 |
| 45.16129 | 91.66667 |
| 94.73684 | 100 |
| 76.47059 | 100 |
| 92.85714 | 95.65217 |
| 70 | 95.55556 |
| 50 | 94.73684 |
| 77.77778 | 77.77778 |
| 96.42857 | 69.23077 |
| 82.35294 | 100 |
| 76.47059 | 93.75 |
| 100 | 100 |
| 100 | 100 |
| 100 | 92.85714 |
| 84.61538 | 75 |
| 75 | 78.94737 |
| 77.77778 | 100 |
| 78.94737 | 100 |
| 100 | 100 |
| 44 | 100 |
| 28.57143 | 94.11765 |
| 60 | 72 |
| 72 | 80.76923 |
| 0 | 72.72727 |
| 85.71429 | 70.58824 |
| 62.5 | 66.66667 |
| 50 | 75 |
| 58.33333 | 97.05882 |
| 70.58824 | 84 |
| 38.88889 | 81.25 |
| 87.5 | 100 |
| 33.33333 | 100 |
| 86.36364 | 87.5 |
| 82.35294 | 78.78788 |
| 92.85714 | 100 |
| 90.47619 | 100 |
| 83.33333 | 100 |

TABLE 21
Numbers of RET+ neurons per myenteric ganglia of
GDNF-treated and saline-treated control 17-month-old mice

| Saline treated Control | GDNF treated |
|---|---|
| 2 | 5 |
| 0 | 2 |
| 1 | 2 |
| 0 | 2 |
| 2 | 3 |
| 3 | 4 |
| 0 | 2 |
| 1 | 3 |
| 3 | 2 |
| 2 | 2 |
| 0 | 4 |
| 2 | 11 |
| 1 | 4 |
| 2 | 2 |
| 2 | 5 |
| 3 | 3 |
| 1 | 4 |
| 1 | 2 |
| 0 | 4 |
| 1 | 4 |
| 1 | 5 |
| 1 | 1 |
| 1 | 2 |
| 0 | 1 |
| 1 | 4 |
| 1 | 4 |
| 0 | 1 |
| 1 | 0 |
| 1 | 0 |
| 2 | 3 |
| 2 | 2 |
| 2 | 4 |
| 1 | 4 |
| 2 | 7 |
| 2 | 5 |
| 1 | 4 |
| 2 | 0 |
| 0 | 5 |
| 2 | 4 |
| 4 | 2 |
| 1 | 3 |
| 2 | |

TABLE 22
scRNAseq annotations

| | | | | | Cell size |
|---|---|---|---|---|---|
| | | | | | normalized |
| | | Gene short | Fraction | | mean |
| Cell group | gene_id | name | expressing | specificity | expression |
| B | ENSMUSG00000000903.3 | Vpreb3 | 0.313253012 | 0.809057247 | 0.401235574 |
| Lymphocytes | | | | | |
| B | ENSMUSG00000003379.7 | Cd79a | 0.861445783 | 0.944306865 | 2.528479914 |
| Lymphocytes | | | | | |
| B | ENSMUSG00000008668.15 | Rps18 | 0.963855422 | 0.207872255 | 7.016180612 |
| Lymphocytes | | | | | |
| B | ENSMUSG00000027368.6 | Dusp2 | 0.674698795 | 0.601583322 | 1.479010504 |
| Lymphocytes | | | | | |
| B | ENSMUSG00000030707.15 | Coro1a | 0.765060241 | 0.283795748 | 1.606489167 |
| Lymphocytes | | | | | |
| B | ENSMUSG00000030724.7 | Cd19 | 0.240963855 | 0.939404404 | 0.252212489 |
| Lymphocytes | | | | | |
| B | ENSMUSG00000030798.15 | Cd37 | 0.644578313 | 0.459780901 | 0.895786503 |
| Lymphocytes | | | | | |
| B | ENSMUSG00000008193.13 | Spit | 0.234939759 | 0.907913394 | 0.232529702 |
| Lymphocytes | | | | | |
| B | ENSMUSG00000036478.8 | Btg1 | 0.873493976 | 0.238854762 | 3.011509467 |
| Lymphocytes | | | | | |
| B | ENSMUSG00000042474.6 | Fcmr | 0.475903614 | 0.927165329 | 0.766150523 |
| Lymphocytes | | | | | |
| B | ENSMUSG00000034634.7 | Ly6d | 0.710843373 | 0.905823998 | 2.117033672 |
| Lymphocytes | | | | | |
| B | ENSMUSG00000037548.15 | H2-DMb2 | 0.63253012 | 0.655855371 | 1.096253407 |
| Lymphocytes | | | | | |
| B | ENSMUSG00000038274.13 | Fau | 0.969879518 | 0.208192304 | 4.687890866 |
| Lymphocytes | | | | | |
| B | ENSMUSG00000040592.11 | Cd79b | 0.78313253 | 0.735306453 | 1.53785567 |
| Lymphocytes | | | | | |
| B | ENSMUSG00000038421.13 | Fcrla | 0.28313253 | 0.816060613 | 0.320703862 |
| Lymphocytes | | | | | |
| B | ENSMUSG00000024610.15 | Cd74 | 1 | 0.193488594 | 31.96604084 |
| Lymphocytes | | | | | |
| B | ENSMUSG00000045128.9 | Rpl18a | 0.963855422 | 0.214922122 | 8.177523222 |
| Lymphocytes | | | | | |
| B | ENSMUSG00000052837.6 | Junb | 0.855421687 | 0.244424588 | 5.815407162 |
| Lymphocytes | | | | | |
| B | ENSMUSG00000057098.14 | Ebf1 | 0.789156627 | 0.393965217 | 2.048617941 |
| Lymphocytes | | | | | |
| B | ENSMUSG00000057841.5 | Rpl32 | 0.981927711 | 0.196559987 | 6.83869643 |
| Lymphocytes | | | | | |
| B | ENSMUSG00000067288.13 | Rps28 | 0.951807229 | 0.210324704 | 5.531622122 |
| Lymphocytes | | | | | |
| B | ENSMUSG00000068105.11 | Tnfrsf13c | 0.403614458 | 0.84431692 | 0.460949381 |
| Lymphocytes | | | | | |
| B | ENSMUSG00000037944.8 | Cer7 | 0.271084337 | 0.72425567 | 0.356238275 |
| Lymphocytes | | | | | |
| B | ENSMUSG00000076617.9 | Ighm | 0.753012048 | 0.572190748 | 2.499663141 |
| Lymphocytes | | | | | |
| B | ENSMUSG00000076937.3 | Iglc2 | 0.578313253 | 0.878673799 | 1.502728801 |
| Lymphocytes | | | | | |
| B | ENSMUSG00000040952.16 | Rps19 | 0.969879518 | 0.22279478 | 10.35352077 |
| Lymphocytes | | | | | |
| B | ENSMUSG00000021754.17 | Map3k1 | 0.698795181 | 0.272698428 | 1.375370808 |
| Lymphocytes | | | | | |
| B | ENSMUSG00000025290.17 | Rps24 | 0.957831325 | 0.201228459 | 5.477858435 |
| Lymphocytes | | | | | |
| B | ENSMUSG00000090862.3 | Rps13 | 0.86746988 | 0.218902519 | 2.043776289 |
| Lymphocytes | | | | | |
| B | ENSMUSG00000104213.5 | Ighd | 0.301204819 | 0.85972184 | 0.433233297 |
| Lymphocytes | | | | | |
| Unknown | ENSMUSG00000007892.8 | Rplp1 | 0.774739583 | 0.154681253 | 4.770952582 |
| Unknown | ENSMUSG00000022108.8 | Itm2b | 0.734809028 | 0.157858962 | 4.920508514 |
| Unknown | ENSMUSG00000024397.14 | Aif1 | 0.5 | 0.437452309 | 2.106594516 |
| Unknown | ENSMUSG00000024661.7 | Fth1 | 0.969184028 | 0.192310639 | 15.5966128 |
| Unknown | ENSMUSG00000027447.6 | Cst3 | 0.860243056 | 0.197404267 | 7.996041782 |
| Unknown | ENSMUSG00000029373.7 | Pf4 | 0.58984375 | 0.341991268 | 3.768609977 |
| Unknown | ENSMUSG00000030579.10 | Tyrobp | 0.641059028 | 0.295908596 | 3.129838819 |
| Unknown | ENSMUSG00000034892.8 | Rps29 | 0.86328125 | 0.169046832 | 7.550035886 |
| Unknown | ENSMUSG00000036594.15 | H2-Aa | 0.861979167 | 0.233166174 | 10.39372253 |
| Unknown | ENSMUSG00000073421.6 | H2-Ab1 | 0.855034722 | 0.210561126 | 11.17139155 |
| Unknown | ENSMUSG00000038274.13 | Fau | 0.66015625 | 0.152986347 | 3.124348505 |
| Unknown | ENSMUSG00000036887.5 | C1qa | 0.801649306 | 0.305938879 | 6.694527219 |
| Unknown | ENSMUSG00000036896.5 | C1qc | 0.6796875 | 0.287510582 | 4.083982542 |
| Unknown | ENSMUSG00000036905.8 | C1qb | 0.794704861 | 0.309196948 | 6.144852291 |
| Unknown | ENSMUSG00000024610.15 | Cd74 | 0.977864583 | 0.241281853 | 42.80526816 |
| Unknown | ENSMUSG00000046330.10 | Rpl37a | 0.821180556 | 0.130125111 | 6.096944191 |
| Unknown | ENSMUSG00000060586.11 | H2-Eb1 | 0.898003472 | 0.259731898 | 11.52976704 |
| Unknown | ENSMUSG00000060636.14 | Rpl35a | 0.708767361 | 0.152328294 | 3.561047524 |
| Unknown | ENSMUSG00000058715.11 | Fcer1g | 0.709635417 | 0.295449224 | 4.180131581 |
| Unknown | ENSMUSG00000064339.1 | mt-Rnr2 | 0.876302083 | 0.144880602 | 9.279961456 |
| Unknown | ENSMUSG00000064341.1 | mt-Nd1 | 0.990017361 | 0.148306435 | 19.99142039 |
| Unknown | ENSMUSG00000064345.1 | mt-Nd2 | 0.802951389 | 0.153246297 | 5.18578848 |
| Unknown | ENSMUSG00000064358.1 | mt-Co3 | 0.776909722 | 0.148697466 | 4.800110863 |
| Unknown | ENSMUSG00000064363.1 | mt-Nd4 | 0.957465278 | 0.154730996 | 12.04068479 |
| Unknown | ENSMUSG00000064370.1 | mt-Cytb | 0.995659722 | 0.146154536 | 23.26826193 |
| Unknown | ENSMUSG00000069516.8 | Lyz2 | 0.840711806 | 0.204584662 | 9.934959093 |
| Unknown | ENSMUSG00000050708.16 | Ftl1 | 0.966145833 | 0.239263265 | 16.68168957 |
| Unknown | ENSMUSG00000073412.6 | Lst1 | 0.352864583 | 0.382996142 | 1.164106735 |
| Unknown | ENSMUSG00000029580.14 | Actb | 0.817708333 | 0.147577492 | 14.17578879 |
| Unknown | ENSMUSG00000049775.16 | Tmsb4x | 0.993489583 | 0.222858594 | 36.82970016 |
| Macrophage-1 | ENSMUSG00000035352.3 | Ccl12 | 0.625714286 | 0.486284309 | 1.694207933 |
| Macrophage-1 | ENSMUSG00000004730.15 | Adgre1 | 0.840571429 | 0.438660334 | 0.943846687 |
| Macrophage-1 | ENSMUSG00000021939.8 | Ctsb | 0.987428571 | 0.30302139 | 3.781356295 |
| Macrophage-1 | ENSMUSG00000038642.10 | Ctss | 0.974285714 | 0.358998356 | 2.761005273 |
| Macrophage-1 | ENSMUSG00000021190.14 | Lgmn | 0.941714286 | 0.346725697 | 1.760177277 |
| Macrophage-1 | ENSMUSG00000021423.6 | Ly86 | 0.825714286 | 0.341954497 | 0.852479568 |
| Macrophage-1 | ENSMUSG00000024621.16 | Csf1r | 0.942857143 | 0.47328469 | 1.450623036 |
| Macrophage-1 | ENSMUSG00000024672.11 | Ms4a7 | 0.842857143 | 0.442050201 | 1.12531841 |
| Macrophage-1 | ENSMUSG00000024677.13 | Ms4a6b | 0.780571429 | 0.391407486 | 0.762380576 |
| Macrophage-1 | ENSMUSG00000026712.3 | Mrc1 | 0.742857143 | 0.53550434 | 0.765293495 |
| Macrophage-1 | ENSMUSG00000030560.17 | Ctsc | 0.962857143 | 0.441106403 | 2.083585476 |
| Macrophage-1 | ENSMUSG00000032359.14 | Ctsh | 0.904571429 | 0.342594294 | 1.186791129 |
| Macrophage-1 | ENSMUSG00000032554.15 | Trf | 0.934285714 | 0.352453003 | 2.230978813 |
| Macrophage-1 | ENSMUSG00000036594.15 | H2-Aa | 0.990857143 | 0.324992002 | 16.11054845 |
| Macrophage-1 | ENSMUSG00000073421.6 | H2-Ab1 | 0.995428571 | 0.333263367 | 20.48034711 |
| Macrophage-1 | ENSMUSG00000040552.8 | C3ar1 | 0.606857143 | 0.502867867 | 0.44391317 |
| Macrophage-1 | ENSMUSG00000036887.5 | C1qa | 0.998285714 | 0.416584133 | 9.984513213 |
| Macrophage-1 | ENSMUSG00000036896.5 | C1qc | 0.996571429 | 0.420737085 | 6.693873454 |
| Macrophage-1 | ENSMUSG00000036905.8 | C1qb | 0.997142857 | 0.398533914 | 8.54359707 |
| Macrophage-1 | ENSMUSG00000024610.15 | Cd74 | 0.999428571 | 0.300700144 | 57.25941989 |
| Macrophage-1 | ENSMUSG00000049130.6 | C5ar1 | 0.796571429 | 0.553760201 | 0.955740085 |
| Macrophage-1 | ENSMUSG00000051439.7 | Cd14 | 0.675428571 | 0.43541697 | 0.575911337 |
| Macrophage-1 | ENSMUSG00000052160.7 | Pld4 | 0.790857143 | 0.381223991 | 0.738163064 |
| Macrophage-1 | ENSMUSG00000055541.17 | Lair1 | 0.592 | 0.536991584 | 0.416338025 |
| Macrophage-1 | ENSMUSG00000055435.6 | Maf | 0.846285714 | 0.372734132 | 1.07592894 |
| Macrophage-1 | ENSMUSG00000060586.11 | H2-Eb1 | 0.992 | 0.331995306 | 15.93121184 |
| Macrophage-1 | ENSMUSG00000074622.4 | Mafb | 0.754857143 | 0.42978119 | 0.769250198 |
| Macrophage-1 | ENSMUSG00000079547.4 | H2-DMb1 | 0.844 | 0.368940844 | 1.122578237 |
| Macrophage-1 | ENSMUSG00000036908.17 | Unc93b1 | 0.92 | 0.366867729 | 1.276242731 |
| Macrophage-1 | ENSMUSG00000059498.13 | Fcgr3 | 0.935428571 | 0.401714511 | 1.521685543 |
| Macrophage-2 | ENSMUSG00000000290.13 | Itgb2 | 0.714285714 | 0.387415492 | 0.513287548 |
| Macrophage-2 | ENSMUSG00000000594.7 | Gm2a | 0.979591837 | 0.388125769 | 2.211237887 |
| Macrophage-2 | ENSMUSG00000001020.8 | S100a4 | 0.693877551 | 0.344013177 | 0.524178156 |
| Macrophage-2 | ENSMUSG00000001128.7 | Cfp | 0.816326531 | 0.586510193 | 1.249862687 |
| Macrophage-2 | ENSMUSG00000004207.14 | Psap | 1 | 0.241893904 | 2.929920321 |
| Macrophage-2 | ENSMUSG00000057729.12 | Prtn3 | 0.265306122 | 0.951054268 | 0.191595857 |
| Macrophage-2 | ENSMUSG00000040026.8 | Saa3 | 0.306122449 | 0.829614085 | 1.269176682 |
| Macrophage-2 | ENSMUSG00000009687.14 | Fxyd5 | 0.93877551 | 0.297554554 | 0.987184713 |
| Macrophage-2 | ENSMUSG00000031494.7 | Cd209a | 0.387755102 | 0.872116814 | 0.732047393 |
| Macrophage-2 | ENSMUSG00000013974.3 | Mcemp1 | 0.408163265 | 0.816763971 | 0.20421958 |
| Macrophage-2 | ENSMUSG00000038642.10 | Ctss | 0.959183673 | 0.247875572 | 1.69649836 |
| Macrophage-2 | ENSMUSG00000015854.7 | Cd51 | 0.306122449 | 0.908120203 | 0.545442392 |
| Macrophage-2 | ENSMUSG00000019122.8 | Ccl9 | 0.734693878 | 0.572357755 | 0.911161858 |
| Macrophage-2 | ENSMUSG00000020077.14 | Srgn | 0.918367347 | 0.260129825 | 0.950622062 |
| Macrophage-2 | ENSMUSG00000061100.4 | Retnla | 0.306122449 | 0.823945277 | 8.05006265 |
| Macrophage-2 | ENSMUSG00000025330.6 | Padi4 | 0.265306122 | 0.900014394 | 0.14431571 |
| Macrophage-2 | ENSMUSG00000027447.6 | Cst3 | 0.979591837 | 0.244473052 | 10.61079263 |
| Macrophage-2 | ENSMUSG00000028581.17 | Laptm5 | 1 | 0.243402329 | 1.05384268 |
| Macrophage-2 | ENSMUSG00000030142.10 | Clec4e | 0.306122449 | 0.914109972 | 0.104079199 |
| Macrophage-2 | ENSMUSG00000031444.16 | F10 | 0.326530612 | 0.943633505 | 0.155957294 |
| Macrophage-2 | ENSMUSG00000031827.13 | Cotl1 | 0.959183673 | 0.28711474 | 1.390786945 |
| Macrophage-2 | ENSMUSG00000032265.14 | Tent5a | 0.795918367 | 0.307578588 | 0.911304235 |
| Macrophage-2 | ENSMUSG00000037894.13 | H2afz | 0.918367347 | 0.266987519 | 0.65338481 |
| Macrophage-2 | ENSMUSG00000037649.10 | H2-DMa | 0.897959184 | 0.279761363 | 1.47251077 |
| Macrophage-2 | ENSMUSG00000060063.9 | Alox5ap | 0.93877551 | 0.495368061 | 1.2393466 |
| Macrophage-2 | ENSMUSG00000059108.4 | Ifitm6 | 0.673469388 | 0.783993527 | 0.36159284 |
| Macrophage-2 | ENSMUSG00000063856.7 | Gpx1 | 1 | 0.30178749 | 2.047491344 |
| Macrophage-2 | ENSMUSG00000069515.6 | Lyz1 | 0.346938776 | 0.813684346 | 1.212289453 |
| Macrophage-2 | ENSMUSG00000069516.8 | Lyz2 | 0.959183673 | 0.452770028 | 27.99314498 |
| Macrophage-2 | ENSMUSG00000069792.5 | Wfdc17 | 0.714285714 | 0.588113696 | 5.077651366 |
| MENS | ENSMUSG00000001025.8 | S100a6 | 0.977507872 | 0.388887178 | 13.04143215 |
| MENS | ENSMUSG00000002980.14 | Bcam | 0.668016194 | 0.411924871 | 1.235315322 |
| MENS | ENSMUSG00000006360.11 | Crip1 | 0.995051732 | 0.317255059 | 29.21014893 |
| MENS | ENSMUSG00000020911.14 | Krt19 | 0.747638327 | 0.480461646 | 2.249780376 |
| MENS | ENSMUSG00000008540.11 | Mgst1 | 0.67431399 | 0.43564887 | 1.35397346 |
| MENS | ENSMUSG00000009281.6 | Rarres2 | 0.973459289 | 0.457047445 | 13.39913582 |
| MENS | ENSMUSG00000020467.15 | Efemp1 | 0.713450292 | 0.378834822 | 1.654679918 |
| MENS | ENSMUSG00000022037.15 | Clu | 0.709401709 | 0.493575739 | 1.552562059 |
| MENS | ENSMUSG00000023046.6 | Igfbp6 | 0.97705803 | 0.530166578 | 15.35188735 |
| MENS | ENSMUSG00000024164.15 | C3 | 0.895636527 | 0.540809865 | 9.54134434 |
| MENS | ENSMUSG00000090231.10 | Cfb | 0.498425551 | 0.552364435 | 0.43212217 |
| MENS | ENSMUSG00000028583.14 | Pdpn | 0.564102564 | 0.475955552 | 0.715995563 |
| MENS | ENSMUSG00000028871.7 | Rspo1 | 0.647323437 | 0.601745723 | 1.204743745 |
| MENS | ENSMUSG00000029718.14 | Pcolce | 0.747638327 | 0.370425313 | 2.046818972 |
| MENS | ENSMUSG00000031517.8 | Gpm6a | 0.763832659 | 0.598484403 | 2.194170934 |
| MENS | ENSMUSG00000013584.5 | Aldh1a2 | 0.549707602 | 0.489258489 | 0.58279337 |
| MENS | ENSMUSG00000060962.12 | Dmkn | 0.450292398 | 0.601427334 | 0.526619949 |
| MENS | ENSMUSG00000040170.13 | Fmo2 | 0.673864148 | 0.481411687 | 1.55450864 |
| MENS | ENSMUSG00000043110.2 | Lrrn4 | 0.526315789 | 0.631190514 | 0.50685094 |
| MENS | ENSMUSG00000049436.5 | Upk1b | 0.614035088 | 0.596402975 | 0.851251111 |
| MENS | ENSMUSG00000042985.7 | Upk3b | 0.791722897 | 0.591116858 | 2.662874316 |
| MENS | ENSMUSG00000023039.17 | Krt7 | 0.646423752 | 0.580578305 | 1.147235982 |
| MENS | ENSMUSG00000055653.13 | Gpc3 | 0.697705803 | 0.439543849 | 1.432580043 |
| MENS | ENSMUSG00000063011.7 | Msln | 0.809716599 | 0.568096908 | 3.105090967 |
| MENS | ENSMUSG00000018339.11 | Gpx3 | 0.811965812 | 0.359604422 | 3.043930066 |
| MENS | ENSMUSG00000020473.13 | Aebp1 | 0.802968961 | 0.534668672 | 3.53374537 |
| MENS | ENSMUSG00000027574.15 | Nkain4 | 0.651821862 | 0.501249597 | 1.13818468 |
| MENS | ENSMUSG00000019929.16 | Dcn | 0.943769681 | 0.286165861 | 13.84099466 |
| MENS | ENSMUSG00000035930.9 | Chst4 | 0.502474134 | 0.585781723 | 0.39772752 |
| MENS | ENSMUSG00000017002.14 | Slpi | 0.992352677 | 0.545415072 | 36.21072514 |
| NC-cells | ENSMUSG00000014846.12 | Tppp3 | 0.740805604 | 0.412367245 | 2.527830966 |
| NC-cells | ENSMUSG00000018593.13 | Sparc | 0.861646235 | 0.318224233 | 6.179055323 |
| NC-cells | ENSMUSG00000019874.11 | Fabp7 | 0.426152948 | 0.524773874 | 0.68020198 |
| NC-cells | ENSMUSG00000020774.9 | Aspa | 0.423817863 | 0.549351363 | 0.629643875 |
| NC-cells | ENSMUSG00000022103.10 | Gfra2 | 0.356100409 | 0.590616295 | 0.446017847 |
| NC-cells | ENSMUSG00000022324.15 | Matn2 | 0.459427904 | 0.538325499 | 0.801693561 |
| NC-cells | ENSMUSG00000022548.14 | Apod | 0.449503795 | 0.573598519 | 1.134911153 |
| NC-cells | ENSMUSG00000024665.8 | Fads2 | 0.408639813 | 0.511650408 | 0.608287744 |
| NC-cells | ENSMUSG00000025780.7 | Itih5 | 0.511967309 | 0.461275986 | 0.930469643 |
| NC-cells | ENSMUSG00000026249.10 | Serpine2 | 0.570928196 | 0.453394906 | 1.13421643 |
| NC-cells | ENSMUSG00000026385.16 | Dbi | 0.886748395 | 0.419286858 | 7.003062368 |
| NC-cells | ENSMUSG00000026424.8 | Gpr37l1 | 0.564506713 | 0.633945422 | 1.11580317 |
| NC-cells | ENSMUSG00000030342.8 | Cd9 | 0.796847636 | 0.362602877 | 3.515288364 |
| NC-cells | ENSMUSG00000031425.15 | Plp1 | 0.650321074 | 0.698316616 | 1.671518165 |
| NC-cells | ENSMUSG00000031548.7 | Sfrp1 | 0.506713368 | 0.408830963 | 1.000987519 |
| NC-cells | ENSMUSG00000032060.10 | Cryab | 0.720373614 | 0.489635832 | 2.264057788 |
| NC-cells | ENSMUSG00000033208.7 | S100b | 0.500291886 | 0.607380839 | 0.804063359 |
| NC-cells | ENSMUSG00000036570.14 | Fxyd1 | 0.821949796 | 0.519741111 | 3.988520799 |
| NC-cells | ENSMUSG00000032679.12 | Cd59a | 0.49737303 | 0.476093508 | 0.796573118 |
| NC-cells | ENSMUSG00000036169.6 | Sostdc1 | 0.524810274 | 0.718129937 | 1.14110985 |
| NC-cells | ENSMUSG00000034810.7 | Scn7a | 0.730881494 | 0.627698665 | 2.400992777 |
| NC-cells | ENSMUSG00000025856.15 | Pdgfa | 0.514886165 | 0.500924516 | 0.941287853 |
| NC-cells | ENSMUSG00000041607.17 | Mbp | 0.364856976 | 0.556870384 | 0.52339682 |
| NC-cells | ENSMUSG00000047976.4 | Kcna1 | 0.51255108 | 0.747208669 | 0.983982492 |
| NC-cells | ENSMUSG00000031342.17 | Gpm6b | 0.361354349 | 0.584925792 | 0.484120611 |
| NC-cells | ENSMUSG00000032268.13 | Tmprss5 | 0.359603036 | 0.656897925 | 0.520418096 |
| NC-cells | ENSMUSG00000030701.17 | Plekhb1 | 0.397548161 | 0.538970787 | 0.542021784 |
| NC-cells | ENSMUSG00000047216.8 | Cdh19 | 0.349095155 | 0.683560149 | 0.452294873 |
| NC-cells | ENSMUSG00000034842.16 | Art3 | 0.549328663 | 0.635916245 | 1.017314589 |
| NC-cells | ENSMUSG00000039542.16 | Ncam1 | 0.647402218 | 0.677846722 | 1.690474178 |
| NK cells | ENSMUSG00000003882.5 | Il7r | 0.532467532 | 0.861353135 | 0.674578092 |
| NK cells | ENSMUSG00000008668.15 | Rps18 | 0.935064935 | 0.200504265 | 6.689358564 |
| NK cells | ENSMUSG00000017404.12 | Rpl19 | 0.883116883 | 0.198941278 | 3.67204039 |
| NK cells | ENSMUSG00000020644.9 | Id2 | 0.831168831 | 0.409481708 | 2.292338863 |
| NK cells | ENSMUSG00000021728.8 | Emb | 0.480519481 | 0.479690383 | 0.509041517 |
| NK cells | ENSMUSG00000024014.8 | Pim1 | 0.636363636 | 0.290362345 | 1.054202855 |
| NK cells | ENSMUSG00000024399.5 | Ltb | 0.675324675 | 0.485982109 | 1.119677363 |
| NK cells | ENSMUSG00000025647.16 | Shisa5 | 0.805194805 | 0.222138276 | 1.262576184 |
| NK cells | ENSMUSG00000026360.9 | Rgs2 | 0.545454545 | 0.325684807 | 0.851644221 |
| NK cells | ENSMUSG00000030114.8 | Klrg1 | 0.246753247 | 1 | 0.201613431 |
| NK cells | ENSMUSG00000030707.15 | Coro1a | 0.805194805 | 0.265703046 | 1.472559526 |
| NK cells | ENSMUSG00000032035.15 | Ets1 | 0.649350649 | 0.273182429 | 0.923608085 |
| NK cells | ENSMUSG00000056290.16 | Ms4a4b | 0.441558442 | 0.719085609 | 1.17558803 |
| NK cells | ENSMUSG00000036478.8 | Btg1 | 0.935064935 | 0.225851695 | 2.796407855 |
| NK cells | ENSMUSG00000039264.9 | Gimap3 | 0.623376623 | 0.486457291 | 0.733861641 |
| NK cells | ENSMUSG00000040747.9 | Cd53 | 0.714285714 | 0.272154429 | 1.14831831 |
| NK cells | ENSMUSG00000037742.14 | Eef1a1 | 0.974025974 | 0.183351869 | 5.712709967 |
| NK cells | ENSMUSG00000033220.7 | Rac2 | 0.701298701 | 0.255165685 | 1.076717562 |
| NK cells | ENSMUSG00000026069.15 | Il1rl1 | 0.298701299 | 0.771580572 | 0.368253574 |
| NK cells | ENSMUSG00000052837.6 | Junb | 0.883116883 | 0.280097789 | 6.963660526 |
| NK cells | ENSMUSG00000057058.16 | Skap1 | 0.363636364 | 0.570331483 | 0.347587958 |
| NK cells | ENSMUSG00000060550.16 | H2-Q7 | 0.597402597 | 0.289958473 | 0.820524814 |
| NK cells | ENSMUSG00000064109.8 | Hcst | 0.545454545 | 0.384431119 | 0.710302744 |
| NK cells | ENSMUSG00000079845.8 | Xlr4a | 0.272727273 | 0.649553028 | 0.192285061 |
| NK cells | ENSMUSG00000067274.10 | Rplp0 | 0.935064935 | 0.194064891 | 5.046773765 |
| NK cells | ENSMUSG00000015619.10 | Gata3 | 0.428571429 | 0.802978212 | 0.867466991 |
| NK cells | ENSMUSG00000076498.2 | Trbc2 | 0.649350649 | 0.331114042 | 0.987419637 |
| NK cells | ENSMUSG00000000486.13 | Sept1 | 0.584415584 | 0.386152804 | 0.669793526 |
| NK cells | ENSMUSG00000032011.5 | Thy1 | 0.480519481 | 0.455025646 | 0.608110384 |
| NK cells | ENSMUSG00000025794.9 | Rpl14 | 0.961038961 | 0.185689924 | 4.303369444 |
| Fibroblasts 1 | ENSMUSG00000029231.15 | Pdgfra | 0.92481203 | 0.52112479 | 1.598198364 |
| Fibroblasts 1 | ENSMUSG00000022860.15 | Chodl | 0.751879699 | 0.659697887 | 0.840766684 |
| Fibroblasts 1 | ENSMUSG00000022890.13 | Atp5j | 0.984962406 | 0.378297819 | 5.927392094 |
| Fibroblasts 1 | ENSMUSG00000023886.10 | Smoc2 | 0.92481203 | 0.481588014 | 1.898781331 |
| Fibroblasts 1 | ENSMUSG00000023913.17 | Pla2g7 | 0.819548872 | 0.467641654 | 0.859728787 |
| Fibroblasts 1 | ENSMUSG00000025488.9 | Cox8b | 0.646616541 | 0.637936959 | 0.533665485 |
| Fibroblasts 1 | ENSMUSG00000025491.14 | Ifitm1 | 0.77443609 | 0.619786608 | 1.044380976 |
| Fibroblasts 1 | ENSMUSG00000026574.5 | Dpt | 0.661654135 | 0.549082626 | 0.510052961 |
| Fibroblasts 1 | ENSMUSG00000026678.10 | Rgs5 | 0.932330827 | 0.427188223 | 2.396915434 |
| Fibroblasts 1 | ENSMUSG00000027210.20 | Meis2 | 0.77443609 | 0.44400719 | 0.871349078 |
| Fibroblasts 1 | ENSMUSG00000028005.13 | Gucy1b1 | 0.864661654 | 0.660263872 | 0.990274746 |
| Fibroblasts 1 | ENSMUSG00000028364.15 | Tnc | 0.706766917 | 0.503330198 | 0.768348653 |
| Fibroblasts 1 | ENSMUSG00000029163.9 | Emilin1 | 0.887218045 | 0.523714612 | 1.046699974 |
| Fibroblasts 1 | ENSMUSG00000030218.2 | Mgp | 0.969924812 | 0.514429679 | 3.8304982 |
| Fibroblasts 1 | ENSMUSG00000056973.6 | Ces1d | 0.69924812 | 0.723204274 | 1.448660691 |
| Fibroblasts 1 | ENSMUSG00000032024.10 | Clmp | 0.766917293 | 0.497502881 | 0.661200751 |
| Fibroblasts 1 | ENSMUSG00000036446.5 | Lum | 0.992481203 | 0.606897557 | 10.08621156 |
| Fibroblasts 1 | ENSMUSG00000039252.11 | Lgi2 | 0.714285714 | 0.594491825 | 0.60582866 |
| Fibroblasts 1 | ENSMUSG00000042436.12 | Mfap4 | 0.834586466 | 0.683295661 | 2.431508045 |
| Fibroblasts 1 | ENSMUSG00000033910.13 | Gucy1a1 | 0.84962406 | 0.598547698 | 1.123589141 |
| Fibroblasts 1 | ENSMUSG00000036766.12 | Dner | 0.518796992 | 0.672235516 | 0.444492553 |
| Fibroblasts 1 | ENSMUSG00000006369.14 | Fbln1 | 0.909774436 | 0.49855092 | 1.439662782 |
| Fibroblasts 1 | ENSMUSG00000058620.5 | Adra2b | 0.721804511 | 0.440170565 | 0.715557048 |
| Fibroblasts 1 | ENSMUSG00000057280.15 | Musk | 0.654135338 | 0.694482985 | 0.737356769 |
| Fibroblasts 1 | ENSMUSG00000066705.7 | Fxyd6 | 0.92481203 | 0.539819849 | 1.779229838 |
| Fibroblasts 1 | ENSMUSG00000053062.16 | Jam2 | 0.77443609 | 0.449703733 | 0.751319526 |
| Fibroblasts 1 | ENSMUSG00000074971.4 | Fibin | 0.909774436 | 0.658316861 | 2.573938036 |
| Fibroblasts 1 | ENSMUSG00000029838.11 | Ptn | 0.992481203 | 0.539243336 | 4.256245839 |
| Fibroblasts 1 | ENSMUSG00000021943.7 | Gdf10 | 0.766917293 | 0.446954345 | 1.140626052 |
| Fibroblasts 1 | ENSMUSG00000023885.9 | Thbs2 | 0.969924812 | 0.643731881 | 1.67387983 |
| Fibroblasts 2 | ENSMUSG00000020676.2 | Ccl11 | 0.617021277 | 0.729102423 | 0.910209791 |
| Fibroblasts 2 | ENSMUSG00000000753.15 | Serpinf1 | 0.617021277 | 0.587381464 | 0.655658251 |
| Fibroblasts 2 | ENSMUSG00000001506.10 | Col1a1 | 0.85106383 | 0.396170097 | 2.095902458 |
| Fibroblasts 2 | ENSMUSG00000003477.5 | Inmt | 0.457446809 | 0.918733181 | 0.754745164 |
| Fibroblasts 2 | ENSMUSG00000005397.8 | Nid1 | 0.776595745 | 0.503535882 | 1.194236151 |
| Fibroblasts 2 | ENSMUSG00000015354.8 | Pcolce2 | 0.638297872 | 0.892022373 | 0.664506086 |
| Fibroblasts 2 | ENSMUSG00000020363.6 | Gfpt2 | 0.468085106 | 0.713722625 | 0.42229275 |
| Fibroblasts 2 | ENSMUSG00000020810.5 | Cygb | 0.787234043 | 0.490541159 | 1.265216869 |
| Fibroblasts 2 | ENSMUSG00000022032.14 | Scara5 | 0.680851064 | 0.925160335 | 0.619511763 |
| Fibroblasts 2 | ENSMUSG00000022371.16 | Col14al | 0.659574468 | 0.54649606 | 0.900405369 |
| Fibroblasts 2 | ENSMUSG00000022894.6 | Adamts5 | 0.478723404 | 0.713634184 | 0.562367482 |
| Fibroblasts 2 | ENSMUSG00000024076.10 | Vit | 0.446808511 | 0.841852564 | 0.337482258 |
| Fibroblasts 2 | ENSMUSG00000025784.5 | Clec3b | 0.861702128 | 0.583296572 | 3.856157385 |
| Fibroblasts 2 | ENSMUSG00000026478.14 | Lamc1 | 0.70212766 | 0.483831726 | 0.71408703 |
| Fibroblasts 2 | ENSMUSG00000026879.14 | Gsn | 1 | 0.708443179 | 40.37245874 |
| Fibroblasts 2 | ENSMUSG00000027204.13 | Fbn1 | 0.670212766 | 0.706042884 | 1.65820268 |
| Fibroblasts 2 | ENSMUSG00000029661.16 | Col1a2 | 0.914893617 | 0.443211201 | 2.812221838 |
| Fibroblasts 2 | ENSMUSG00000030116.14 | Mfap5 | 0.808510638 | 0.81714454 | 2.016318492 |
| Fibroblasts 2 | ENSMUSG00000064080.12 | Fbln2 | 0.712765957 | 0.461318918 | 0.979636884 |
| Fibroblasts 2 | ENSMUSG00000032334.10 | Loxl1 | 0.627659574 | 0.527987428 | 0.61837028 |
| Fibroblasts 2 | ENSMUSG00000045573.9 | Penk | 0.627659574 | 0.727217674 | 0.947527116 |
| Fibroblasts 2 | ENSMUSG00000056481.7 | Cd248 | 0.617021277 | 0.744124935 | 1.208971295 |
| Fibroblasts 2 | ENSMUSG00000057098.14 | Ebf1 | 0.840425532 | 0.389711231 | 2.020356294 |
| Fibroblasts 2 | ENSMUSG00000033327.17 | Tnxb | 0.829787234 | 0.543784674 | 1.964666078 |
| Fibroblasts 2 | ENSMUSG00000029096.15 | Htra3 | 0.734042553 | 0.666167109 | 1.18882785 |
| Fibroblasts 2 | ENSMUSG00000026043.18 | Col3a1 | 0.968085106 | 0.467945836 | 5.5355663 |
| Fibroblasts 2 | ENSMUSG00000071984.10 | Fndc1 | 0.531914894 | 0.657844137 | 0.652523961 |
| Fibroblasts 2 | ENSMUSG00000024011.17 | Pi16 | 0.468085106 | 0.733079583 | 1.04099087 |
| Fibroblasts 2 | ENSMUSG00000022816.11 | Fstl1 | 0.840425532 | 0.445778159 | 1.596192194 |
| Fibroblasts 2 | ENSMUSG00000038521.17 | C1s1 | 0.79787234 | 0.41840448 | 0.99189846 |
| RBC | ENSMUSG00000006574.15 | Slc4a1 | 0.026509573 | 0.955893117 | 0.005165091 |
| RBC | ENSMUSG00000020295.1 | Hbq1a | 0.058910162 | 0.964742082 | 0.048334582 |
| RBC | ENSMUSG00000022051.15 | Bnip3l | 0.338733432 | 0.117060954 | 0.236472123 |
| RBC | ENSMUSG00000052305.6 | Hbb-bs | 0.977908689 | 0.818165133 | 358.8726257 |
| RBC | ENSMUSG00000024588.9 | Fech | 0.23269514 | 0.204061205 | 0.09356318 |
| RBC | ENSMUSG00000029922.15 | Mkrn1 | 0.357879234 | 0.206342619 | 0.289333331 |
| RBC | ENSMUSG00000034248.7 | Slc25a37 | 0.194403535 | 0.357601083 | 0.123447866 |
| RBC | ENSMUSG00000039236.18 | Isg20 | 0.207658321 | 0.31716938 | 0.08455282 |
| RBC | ENSMUSG00000038871.5 | Bpgm | 0.382916053 | 0.457146217 | 0.342526656 |
| RBC | ENSMUSG00000044792.7 | Isca1 | 0.204712813 | 0.178036042 | 0.081987151 |
| RBC | ENSMUSG00000051839.7 | Gypa | 0.076583211 | 0.913995233 | 0.057592864 |
| RBC | ENSMUSG00000025270.13 | Alas2 | 0.553755523 | 0.835008556 | 0.647834963 |
| RBC | ENSMUSG00000064339.1 | mt-Rnr2 | 0.736377025 | 0.04821034 | 2.281208831 |
| RBC | ENSMUSG00000064341.1 | mt-Nd1 | 0.957290133 | 0.066265524 | 7.098938331 |
| RBC | ENSMUSG00000064345.1 | mt-Nd2 | 0.702503682 | 0.073565622 | 2.013342264 |
| RBC | ENSMUSG00000064351.1 | mt-Co1 | 0.379970545 | 0.05495388 | 0.65831553 |
| RBC | ENSMUSG00000064357.1 | mt-Atp6 | 0.540500736 | 0.061940657 | 1.02177236 |
| RBC | ENSMUSG00000064358.1 | mt-Co3 | 0.645066274 | 0.059796402 | 1.492676547 |
| RBC | ENSMUSG00000064363.1 | mt-Nd4 | 0.902798233 | 0.073538383 | 4.613842812 |
| RBC | ENSMUSG00000064370.1 | mt-Cytb | 0.976435935 | 0.067769798 | 8.664025757 |
| RBC | ENSMUSG00000069917.7 | Hba-a2 | 0.960235641 | 0.835322244 | 188.6471984 |
| RBC | ENSMUSG00000069919.7 | Hba-a1 | 0.963181149 | 0.82710788 | 209.5761487 |
| RBC | ENSMUSG00000050708.16 | Ftl1 | 0.737849779 | 0.035452004 | 1.448115871 |
| RBC | ENSMUSG00000073940.3 | Hbb-bt | 0.877761414 | 0.850743527 | 41.80691386 |
| RBC | ENSMUSG00000074269.10 | Rec114 | 0.114874816 | 0.573993728 | 0.06734349 |
| RBC | ENSMUSG00000017002.14 | Slpi | 0.471281296 | 0.045525612 | 1.496655216 |
| RBC | ENSMUSG00000078974.10 | Sec61g | 0.293078056 | 0.129985377 | 0.180144951 |
| RBC | ENSMUSG00000025889.13 | Snca | 0.36377025 | 0.714910479 | 0.282474977 |
| RBC | ENSMUSG00000091694.9 | Apol11b | 0.039764359 | 0.710945313 | 0.014773219 |
| RBC | ENSMUSG00000101939.1 | Gm28438 | 0.329896907 | 0.068249497 | 0.53236652 |
| Smooth | ENSMUSG00000005672.12 | Kit | 0.163774403 | 0.592871304 | 0.744073184 |
| muscle cells | | | | | |
| Smooth | ENSMUSG00000020439.17 | Smtn | 0.302603037 | 0.53417354 | 1.443454234 |
| muscle cells | | | | | |
| Smooth | ENSMUSG00000020719.14 | Ddx5 | 0.544468547 | 0.188777946 | 2.584410776 |
| muscle cells | | | | | |
| Smooth | ENSMUSG00000026208.9 | Des | 0.302603037 | 0.35763202 | 1.34731262 |
| muscle cells | | | | | |
| Smooth | ENSMUSG00000028464.16 | Tpm2 | 0.419739696 | 0.362140702 | 2.198294852 |
| muscle cells | | | | | |
| Smooth | ENSMUSG00000032366.15 | Tpm1 | 0.336225597 | 0.308032494 | 1.693205382 |
| muscle cells | | | | | |
| Smooth | ENSMUSG00000030409.15 | Dmpk | 0.235357918 | 0.464273058 | 0.853990395 |
| muscle cells | | | | | |
| Smooth | ENSMUSG00000031328.15 | Flna | 0.386117137 | 0.379685036 | 2.126322145 |
| muscle cells | | | | | |
| Smooth | ENSMUSG00000031586.17 | Rbpms | 0.237527115 | 0.428685963 | 0.803249864 |
| muscle cells | | | | | |
| Smooth | ENSMUSG00000032085.5 | Tagln | 0.206073753 | 0.392158673 | 0.853917456 |
| muscle cells | | | | | |
| Smooth | ENSMUSG00000035967.15 | Ints6l | 0.17462039 | 0.546351891 | 0.602092657 |
| muscle cells | | | | | |
| Smooth | ENSMUSG00000035783.9 | Acta2 | 0.395878525 | 0.411701763 | 2.644535199 |
| muscle cells | | | | | |
| Smooth | ENSMUSG00000041741.10 | Pde3a | 0.218004338 | 0.386569786 | 0.76961166 |
| muscle cells | | | | | |
| Smooth | ENSMUSG00000037852.8 | Cpe | 0.276572668 | 0.334851865 | 1.217302466 |
| muscle cells | | | | | |
| Smooth | ENSMUSG00000059430.14 | Actg2 | 0.250542299 | 0.423160851 | 1.37882212 |
| muscle cells | | | | | |
| Smooth | ENSMUSG00000064339.1 | mt-Rnr2 | 0.859002169 | 0.125798215 | 7.719703149 |
| muscle cells | | | | | |
| Smooth | ENSMUSG00000064341.1 | mt-Nd1 | 0.989154013 | 0.170923024 | 24.08356918 |
| muscle cells | | | | | |
| Smooth | ENSMUSG00000064345.1 | mt-Nd2 | 0.804772234 | 0.180748821 | 6.441992126 |
| muscle cells | | | | | |
| Smooth | ENSMUSG00000064357.1 | mt-Atp6 | 0.638828633 | 0.162274594 | 3.525004248 |
| muscle cells | | | | | |
| Smooth | ENSMUSG00000064358.1 | mt-Co3 | 0.738611714 | 0.163882111 | 5.452777377 |
| muscle cells | | | | | |
| Smooth | ENSMUSG00000064363.1 | mt-Nd4 | 0.956616052 | 0.181640902 | 14.8657575 |
| muscle cells | | | | | |
| Smooth | ENSMUSG00000064370.1 | mt-Cytb | 0.99132321 | 0.176047068 | 29.70486917 |
| muscle cells | | | | | |
| Smooth | ENSMUSG00000067818.6 | Myl9 | 0.386117137 | 0.380541373 | 2.193005633 |
| muscle cells | | | | | |
| Smooth | ENSMUSG00000018830.10 | Myh11 | 0.245119306 | 0.472224391 | 1.180389428 |
| muscle cells | | | | | |
| Smooth | ENSMUSG00000055632.18 | Hmcn2 | 0.317787419 | 0.418184572 | 1.309629655 |
| muscle cells | | | | | |
| Smooth | ENSMUSG00000034275.18 | Igsf9b | 0.253796095 | 0.609969103 | 1.318336759 |
| muscle cells | | | | | |
| Smooth | ENSMUSG00000090841.2 | Myl6 | 0.640997831 | 0.201194419 | 4.14819963 |
| muscle cells | | | | | |
| Smooth | ENSMUSG00000092341.3 | Malat1 | 0.860086768 | 0.094072903 | 41.62379256 |
| muscle cells | | | | | |
| Smooth | ENSMUSG00000097971.3 | Gm26917 | 0.847071584 | 0.199762333 | 22.19320163 |
| muscle cells | | | | | |
| Smooth | ENSMUSG00000101939.1 | Gm28438 | 0.468546638 | 0.202307671 | 2.173270551 |
| muscle cells | | | | | |
| T cells | ENSMUSG00000002033.14 | Cd3g | 0.8875 | 0.751510212 | 3.539364275 |
| T cells | ENSMUSG00000005947.11 | Itgae | 0.6 | 0.876538045 | 0.715196758 |
| T cells | ENSMUSG00000015437.5 | Gzmb | 0.75 | 0.827459804 | 7.499119616 |
| T cells | ENSMUSG00000023132.8 | Gzma | 0.85 | 0.845262796 | 33.41909158 |
| T cells | ENSMUSG00000024910.5 | Ctsw | 0.6875 | 0.65200137 | 0.884192255 |
| T cells | ENSMUSG00000025163.6 | Cd7 | 0.4 | 0.792057067 | 0.315507972 |
| T cells | ENSMUSG00000026826.13 | Nr4a2 | 0.5875 | 0.727407161 | 1.177911932 |
| T cells | ENSMUSG00000030124.2 | Lag3 | 0.4875 | 0.567941171 | 0.488224431 |
| T cells | ENSMUSG00000030165.16 | Klrd1 | 0.6375 | 0.606482383 | 1.669801049 |
| T cells | ENSMUSG00000032094.8 | Cd3d | 0.7625 | 0.663379111 | 1.356300062 |
| T cells | ENSMUSG00000032380.9 | Dapk2 | 0.4875 | 0.598034674 | 0.571937987 |
| T cells | ENSMUSG00000035042.2 | Ccl5 | 0.9625 | 0.766102966 | 48.95590245 |
| T cells | ENSMUSG00000037754.13 | Ppp1r16b | 0.6625 | 0.429364566 | 1.222391384 |
| T cells | ENSMUSG00000038304.14 | Cd160 | 0.3125 | 0.825387037 | 0.317425372 |
| T cells | ENSMUSG00000070691.10 | Runx3 | 0.6 | 0.595378066 | 0.851524197 |
| T cells | ENSMUSG00000053977.13 | Cd8a | 0.775 | 0.911604559 | 1.960209999 |
| T cells | ENSMUSG00000000409.14 | Lck | 0.6375 | 0.625432242 | 0.663362474 |
| T cells | ENSMUSG00000004612.9 | Nkg7 | 0.8 | 0.624325315 | 3.790232357 |
| T cells | ENSMUSG00000068227.9 | I12rb | 0.45 | 0.576717069 | 0.605219506 |
| T cells | ENSMUSG00000070803.6 | Cited4 | 0.4 | 0.741828161 | 0.417102547 |
| T cells | ENSMUSG00000075010.5 | AW112010 | 0.85 | 0.371094509 | 4.474610619 |
| T cells | ENSMUSG00000032093.7 | Cd3e | 0.7125 | 0.761835957 | 1.220219917 |
| T cells | ENSMUSG00000076498.2 | Trbc2 | 0.7125 | 0.544480197 | 1.835970983 |
| T cells | ENSMUSG00000076749.2 | Tcrg-C1 | 0.5125 | 0.693627056 | 1.205351334 |
| T cells | ENSMUSG00000076752.2 | Torg-C2 | 0.4125 | 0.76604286 | 0.514211202 |
| T cells | ENSMUSG00000076928.5 | Trac | 0.65 | 0.596934449 | 1.005364095 |
| T cells | ENSMUSG00000026358.13 | Rgs1 | 0.6875 | 0.631403808 | 1.591858919 |
| T cells | ENSMUSG00000076757.9 | Tcrg-C4 | 0.5 | 0.589507731 | 0.499802433 |
| T cells | ENSMUSG00000100150.2 | Gm19585 | 0.5125 | 0.651851929 | 0.475245827 |
| T cells | ENSMUSG00000114333.1 | 1700084D21Rik | 0.3125 | 0.995173554 | 0.469944922 |
| Vascular | ENSMUSG00000001946.14 | Esam | 0.606593407 | 0.560770851 | 1.204350363 |
| endothelium | | | | | |
| Vascular | ENSMUSG00000004655.5 | Aqp1 | 0.795604396 | 0.66558362 | 7.461814769 |
| endothelium | | | | | |
| Vascular | ENSMUSG00000020577.17 | Tspan13 | 0.667032967 | 0.456250032 | 1.670204018 |
| endothelium | | | | | |
| Vascular | ENSMUSG00000044258.10 | Ctla2a | 0.545054945 | 0.581548863 | 1.101299133 |
| endothelium | | | | | |
| Vascular | ENSMUSG00000022018.7 | Rgcc | 0.558241758 | 0.524713612 | 1.449774171 |
| endothelium | | | | | |
| Vascular | ENSMUSG00000075602.10 | Ly6a | 0.772527473 | 0.421956086 | 2.956659192 |
| endothelium | | | | | |
| Vascular | ENSMUSG00000024140.10 | Epas1 | 0.557142857 | 0.576764652 | 0.955574568 |
| endothelium | | | | | |
| Vascular | ENSMUSG00000025608.9 | Podxl | 0.428571429 | 0.644521903 | 0.590619945 |
| endothelium | | | | | |
| Vascular | ENSMUSG00000062515.3 | Fabp4 | 0.898901099 | 0.66528367 | 10.18820299 |
| endothelium | | | | | |
| Vascular | ENSMUSG00000027800.14 | Tm4sf1 | 0.657142857 | 0.403207686 | 1.635201418 |
| endothelium | | | | | |
| Vascular | ENSMUSG00000029309.7 | Sparcl1 | 0.716483516 | 0.410607074 | 1.976649732 |
| endothelium | | | | | |
| Vascular | ENSMUSG00000029648.13 | Flt1 | 0.471428571 | 0.771478449 | 0.702926261 |
| endothelium | | | | | |
| Vascular | ENSMUSG00000032766.9 | Gng11 | 0.650549451 | 0.467002335 | 1.768630006 |
| endothelium | | | | | |
| Vascular | ENSMUSG00000031871.9 | Cdh5 | 0.659340659 | 0.70108598 | 1.397864592 |
| endothelium | | | | | |
| Vascular | ENSMUSG00000039167.12 | Adgrl4 | 0.385714286 | 0.716472037 | 0.455470225 |
| endothelium | | | | | |
| Vascular | ENSMUSG00000033191.14 | Tie1 | 0.406593407 | 0.658483954 | 0.442417688 |
| endothelium | | | | | |
| Vascular | ENSMUSG00000045092.8 | S1pr1 | 0.545054945 | 0.64227798 | 0.868104913 |
| endothelium | | | | | |
| Vascular | ENSMUSG00000044562.12 | Rasip1 | 0.371428571 | 0.734896052 | 0.427539138 |
| endothelium | | | | | |
| Vascular | ENSMUSG00000020717.19 | Pecam1 | 0.672527473 | 0.710705307 | 1.3929694 |
| endothelium | | | | | |
| Vascular | ENSMUSG00000061353.11 | Cxcl12 | 0.628571429 | 0.752925679 | 2.174326282 |
| endothelium | | | | | |
| Vascular | ENSMUSG00000063415.12 | Cyp26b1 | 0.489010989 | 0.599105151 | 0.986389605 |
| endothelium | | | | | |
| Vascular | ENSMUSG00000002944.15 | Cd36 | 0.795604396 | 0.560432197 | 2.875695868 |
| endothelium | | | | | |
| Vascular | ENSMUSG00000037936.15 | Scarb1 | 0.464835165 | 0.579768602 | 0.68094186 |
| endothelium | | | | | |
| Vascular | ENSMUSG00000068079.5 | Tcf15 | 0.431868132 | 0.747488347 | 0.58673232 |
| endothelium | | | | | |
| Vascular | ENSMUSG00000020154.10 | Ptprb | 0.545054945 | 0.766230625 | 0.950664886 |
| endothelium | | | | | |
| Vascular | ENSMUSG00000027435.8 | Cd93 | 0.501098901 | 0.576519747 | 0.806059959 |
| endothelium | | | | | |
| Vascular | ENSMUSG00000026921.18 | Egfl7 | 0.581318681 | 0.678911985 | 0.963397582 |
| endothelium | | | | | |
| Vascular | ENSMUSG00000062960.10 | Kdr | 0.381318681 | 0.717149335 | 0.449238925 |
| endothelium | | | | | |
| Vascular | ENSMUSG00000056492.6 | Adgrf5 | 0.530769231 | 0.676781278 | 0.861376331 |
| endothelium | | | | | |
| Vascular | ENSMUSG00000054690.17 | Emcn | 0.502197802 | 0.74332006 | 0.76466193 |
| endothelium | | | | | |

### REFERENCES

1 Kulkarni, S. et al. Advances in Enteric Neurobiology: The “Brain” in the Gut in Health and Disease. J Neurosci 38, 9346-9354, doi:10.1523/JNEUROSCI.1663-18.2018 (2018).
2 Jarret, A. et al. Enteric Nervous System-Derived IL-18 Orchestrates Mucosal Barrier Immunity. Cell 180, 50-63 e12, doi:10.1016/j.cell.2019.12.016 (2020).
3 Uesaka, T., Young, H. M., Pachnis, V. & Enomoto, H. Development of the intrinsic and extrinsic innervation of the gut. Dev Biol 417, 158-167, doi:10.1016/j.ydbio.2016.04.016 (2016).
4 Obermayr, F., Hotta, R., Enomoto, H. & Young, H. M. Development and developmental disorders of the enteric nervous system. Nat Rev Gastroenterol Hepatol 10, 43-57, doi:10.1038/nrgastro.2012.234 (2013).
5 Hao, M. M. & Young, H. M. Development of enteric neuron diversity. J Cell Mol Med 13, 1193-1210, doi:10.1111/j.1582-4934.2009.00813.x (2009).
6 Anderson, R. B., Newgreen, D. F. & Young, H. M. Neural crest and the development of the enteric nervous system. Adv Exp Med Biol 589, 181-196, doi:10.1007/978-0-387-46954-6_11 (2006).
7 Young, H. M. & Newgreen, D. Enteric neural crest-derived cells: origin, identification, migration, and differentiation. Anat Rec 262, 1-15, doi:10.1002/1097-0185(20010101)262:1<1:AID-AR1006>3.0.CO; 2-2 (2001).
8 Young, H. M., Hearn, C. J. & Newgreen, D. F. Embryology and development of the enteric nervous system. Gut 47 Suppl 4, iv12-14; discussion iv26, doi:10.1136/gut.47.suppl_4.iv12 (2000).
9 Bergner, A. J. et al. Birthdating of myenteric neuron subtypes in the small intestine of the mouse. J Comp Neurol 522, 514-527, doi:10.1002/cne.23423 (2014).
10 Uesaka, T., Nagashimada, M. & Enomoto, H. Neuronal Differentiation in Schwann Cell Lineage Underlies Postnatal Neurogenesis in the Enteric Nervous System. J Neurosci 35, 9879-9888, doi:10.1523/JNEUROSCI.1239-15.2015 (2015).
11 Espinosa-Medina, I. et al. Dual origin of enteric neurons in vagal Schwann cell precursors and the sympathetic neural crest. Proc Natl Acad Sci USA 114, 11980-11985, doi:10.1073/pnas.1710308114 (2017).
12 Burns, A. J. Migration of neural crest-derived enteric nervous system precursor cells to and within the gastrointestinal tract. Int J Dev Biol 49, 143-150, doi:10.1387/ijdb.041935ab (2005).
13 Laranjeira, C. et al. Glial cells in the mouse enteric nervous system can undergo neurogenesis in response to injury. J Clin Invest 121, 3412-3424, doi:10.1172/JC158200 (2011).
14 Brokhman, I. et al. Dual embryonic origin of the mammalian enteric nervous system. Dev Biol 445, 256-270, doi:10.1016/j.ydbio.2018.11.014 (2019).
Sohal, G. S., Ali, M. M. & Farooqui, F. A. A second source of precursor cells for the developing enteric nervous system and interstitial cells of Cajal. Int J Dev Neurosci 20, 619-626 (2002).
16 Habeck, M. VENT cells—a load of hot air? Drug Discov Today 8, 332-333, doi:10.1016/si359-6446(03)02663-1 (2003).
17 Saffrey, M. J. Cellular changes in the enteric nervous system during ageing. Dev Biol 382, 344-355, doi:10.1016/j.ydbio.2013.03.015 (2013).
18 Becker, L., Kulkarni, S., Tiwari, G., Micci, M. A. & Pasricha, P. J. Divergent fate and origin of neurosphere-like bodies from different layers of the gut. Am J Physiol Gastrointest Liver Physiol 302, G958-965, doi:10.1152/ajpgi.00511.2011 (2012).
19 Antal, M., Beneduce, B. M. & Regehr, W. G. The substantia nigra conveys target-dependent excitatory and inhibitory outputs from the basal ganglia to the thalamus. J Neurosci 34, 8032-8042, doi:10.1523/JNEUROSCI.0236-14.2014 (2014).
Freyer, L., Aggarwal, V. & Morrow, B. E. Dual embryonic origin of the mammalian otic vesicle forming the inner ear. Development 138, 5403-5414, doi:10.1242/dev.069849 (2011).
21 Honig, G., Liou, A., Berger, M., German, M. S. & Tecott, L. H. Precise pattern of recombination in serotonergic and hypothalamic neurons in a Pdx1-cre transgenic mouse line. J Biomed Sci 17, 82, doi:10.1186/1423-0127-17-82 (2010).
22 Bardot, E. et al. Foxa2 identifies a cardiac progenitor population with ventricular differentiation potential. Nat Commun 8, 14428, doi:10.1038/ncomms14428 (2017).
23 Burtscher, I., Barkey, W., Schwarzfischer, M., Theis, F. J. & Lickert, H. The Soxl7-mCherry fusion mouse line allows visualization of endoderm and vascular endothelial development. genesis 50, 496-505, doi:10.1002/dvg.20829 (2012).
24 Wilm, B., Ipenberg, A., Hastie, N. D., Burch, J. B. & Bader, D. M. The serosal mesothelium is a major source of smooth muscle cells of the gut vasculature. Development 132, 5317-5328, doi:10.1242/dev.02141 (2005).
Kawaguchi, M., Bader, D. M. & Wilm, B. Serosal mesothelium retains vasculogenic potential. Dev Dyn 236, 2973-2979, doi:10.1002/dvdy.21334 (2007).
26 Herrick, S. E. & Mutsaers, S. E. Mesothelial progenitor cells and their potential in tissue engineering. Int J Biochem Cell Biol 36, 621-642, doi:10.1016/j.biocel.2003.11.002 (2004).
27 Chan, Sunny S.-K. et al. Mesp1 Patterns Mesoderm into Cardiac, Hematopoietic, or Skeletal Myogenic Progenitors in a Context-Dependent Manner. Cell Stem Cell 12, 587-601, doi:10.1016/j.stem.2013.03.004 (2013).
28 Devine, W. P., Wythe, J. D., George, M., Koshiba-Takeuchi, K. & Bruneau, B. G. Early patterning and specification of cardiac progenitors in gastrulating mesoderm. Elife 3, doi:10.7554/eLife.03848 (2014).
29 Lescroart, F. et al. Early lineage restriction in temporally distinct populations of Mesp1 progenitors during mammalian heart development. Nat Cell Biol 16, 829-840, doi:10.1038/ncb3024 (2014).
Klotz, L. et al. Cardiac lymphatics are heterogeneous in origin and respond to injury. Nature 522, 62-67, doi:10.1038/nature14483 (2015).
31 Chan, S. S. et al. Mesp1 patterns mesoderm into cardiac, hematopoietic, or skeletal myogenic progenitors in a context-dependent manner. Cell Stem Cell 12, 587-601, doi:10.1016/j.stem.2013.03.004 (2013).
32 Baup, D., Moser, M., Schurmans, S. & Leo, O. Developmental regulation of the composite CAG promoter activity in the murine T lymphocyte cell lineage. Genesis 47, 799-804, doi:10.1002/dvg.20569 (2009).
33 Alexopoulou, A. N., Couchman, J. R. & Whiteford, J. R. The CMV early enhancer/chicken beta actin (CAG) promoter can be used to drive transgene expression during the differentiation of murine embryonic stem cells into vascular progenitors. BMC Cell Biol 9, 2, doi:10.1186/1471-2121-9-2 (2008).
34 Hu, X. et al. Reprogramming progressive cells display low CAG promoter activity. Stem Cells 39, 43-54, doi:10.1002/stem.3295 (2021).
Akagi, Y. et al. Transcriptional activation of a hybrid promoter composed of cytomegalovirus enhancer and beta-actin/beta-globin gene in glomerular epithelial cells in vivo. Kidney Int 51, 1265-1269, doi:10.1038/ki.1997.172 (1997).
36 Agah, R. et al. Gene recombination in postmitotic cells. Targeted expression of Cre recombinase provokes cardiac-restricted, site-specific rearrangement in adult ventricular muscle in vivo. J Clin Invest 100, 169-179, doi:10.1172/JCI119509 (1997).
37 Sokoloff, A. J., Li, H. & Burkholder, T. J. Limited expression of slow tonic myosin heavy chain in human cranial muscles. Muscle Nerve 36, 183-189, doi:10.1002/mus.20797 (2007).
38 Stockdale, F. E. & Miller, J. B. The cellular basis of myosin heavy chain isoform expression during development of avian skeletal muscles. Dev Biol 123, 1-9, doi:10.1016/0012-1606(87)90420-9 (1987).
39 Miller, J. B., Crow, M. T. & Stockdale, F. E. Slow and fast myosin heavy chain content defines three types of myotubes in early muscle cell cultures. J Cell Biol 101, 1643-1650, doi:10.1083/jcb.101.5.1643 (1985).
Vaughan, S. K., Stanley, O. L. & Valdez, G. Impact of Aging on Proprioceptive Sensory Neurons and Intrafusal Muscle Fibers in Mice. J Gerontol A Biol Sci Med Sci 72, 771-779, doi:10.1093/gerona/glw175 (2017).
41 Avetisyan, M. et al. Hepatocyte Growth Factor and MET Support Mouse Enteric Nervous System Development, the Peristaltic Response, and Intestinal Epithelial Proliferation in Response to Injury. J Neurosci 35, 11543-11558, doi:10.1523/JNEUROSCI.5267-14.2015 (2015).
42 Rappolee, D. A., Iyer, A. & Patel, Y. Hepatocyte growth factor and its receptor are expressed in cardiac myocytes during early cardiogenesis. Circ Res 78, 1028-1036, doi:10.1161/01.res.78.6.1028 (1996).
43 Mao, Y., Wang, B. & Kunze, W. Characterization of myenteric sensory neurons in the mouse small intestine. J Neurophysiol 96, 998-1010, doi:10.1152/jn.00204.2006 (2006).
44 Abraira, V. E. et al. The Cellular and Synaptic Architecture of the Mechanosensory Dorsal Horn. Cell 168, 295-310 e219, doi:10.1016/j.cell.2016.12.010 (2017).
Zeisel, A. et al. Molecular Architecture of the Mouse Nervous System. Cell 174, 999-1014 e1022, doi:10.1016/j.cell.2018.06.021 (2018).
46 Drokhlyansky, E. et al. The Human and Mouse Enteric Nervous System at Single-Cell Resolution. Cell 182, 1606-1622, doi:10.1016/j.cell.2020.08.00310.1101/746743 (2020).
47 De Giorgio, R., Arakawa, J., Wetmore, C. J. & Sternini, C. Neurotrophin-3 and neurotrophin receptor immunoreactivity in peptidergic enteric neurons. Peptides 21, 1421-1426, doi:10.1016/s0196-9781(00)00286-2 (2000).
48 Barrenschee, M. et al. SNAP-25 is abundantly expressed in enteric neuronal networks and upregulated by the neurotrophic factor GDNF. Histochem Cell Biol 143, 611-623, doi:10.1007/s00418-015-1310-x (2015).
49 Gianino, S., Grider, J. R., Cresswell, J., Enomoto, H. & Heuckeroth, R. O. GDNF availability determines enteric neuron number by controlling precursor proliferation. Development 130, 2187-2198, doi:10.1242/dev.00433 (2003).
50 Natarajan, D., Marcos-Gutierrez, C., Pachnis, V. & de Graaff, E. Requirement of signalling by receptor tyrosine kinase RET for the directed migration of enteric nervous system progenitor cells during mammalian embryogenesis. Development 129, 5151-5160 (2002).
51 Taraviras, S. et al. Signalling by the RET receptor tyrosine kinase and its role in the development of the mammalian enteric nervous system. Development 126, 2785-2797 (1999).
52 Rodrigues, D. M., Li, A. Y., Nair, D. G. & Blennerhassett, M. G. Glial cell line-derived neurotrophic factor is a key neurotrophin in the postnatal enteric nervous system. Neurogastroenterol Motil 23, e44-56, doi:10.1111/j.1365-2982.2010.01626.x (2011).
53 Amano, O. et al. Hepatocyte growth factor is essential for migration of myogenic cells and promotes their proliferation during the early periods of tongue morphogenesis in mouse embryos. Dev Dyn 223, 169-179, doi:10.1002/dvdy.1228 (2002).
54 Wang, H. et al. The timing and location of glial cell line-derived neurotrophic factor expression determine enteric nervous system structure and function. J Neurosci 30, 1523-1538, doi:10.1523/JNEUROSCI.3861-09.2010 (2010).
55 (!!! INVALID CITATION !!!).
56 Uesaka, T., Nagashimada, M., Yonemura, S. & Enomoto, H. Diminished Ret expression compromises neuronal survival in the colon and causes intestinal aganglionosis in mice. J Clin Invest 118, 1890-1898, doi:10.1172/JCI34425 (2008).
57 Kim, M. et al. Obstructed defecation—an enteric neuropathy? An exploratory study of patient samples. Int J Colorectal Dis 34, 193-196, doi:10.1007/s00384-018-3160-1 (2019).
58 Sun, T. et al. Aging-dependent decrease in the numbers of enteric neurons, interstitial cells of Cajal and expression of connexin43 in various regions of gastrointestinal tract. Aging (Albany NY) 10, 3851-3865, doi:10.18632/aging.101677 (2018).
59 Becker, L. et al. Age-dependent shift in macrophage polarisation causes inflammation-mediated degeneration of enteric nervous system. Gut 67, 827-836, doi:10.1136/gutjnl-2016-312940 (2018).
60 Sathyan, S. et al. Plasma proteomic profile of age, health span, and all-cause mortality in older adults. Aging Cell n/a, e13250, doi:10.1111/acel.13250. 61 Stein-O'Brien, G. L. et al. Decomposing Cell Identity for Transfer Learning across Cellular Measurements, Platforms, Tissues, and Species. Cell Syst 8, 395-411 e398, doi:10.1016/j.cels.2019.04.004 (2019).
62 Stein-O'Brien, G. L. et al. Enter the Matrix: Factorization Uncovers Knowledge from Omics. Trends Genet 34, 790-805, doi:10.1016/j.tig.2018.07.003 (2018).
63 Sharma, G., Colantuoni, C., Goff, L. A., Fertig, E. J. & Stein-O'Brien, G. projectR: an R/Bioconductor package for transfer learning via PCA, NMF, correlation and clustering. Bioinformatics 36, 3592-3593, doi:10.1093/bioinformatics/btaa183 (2020).
64 Young, H. M. & Newgreen, D. Enteric neural crest-derived cells: Origin, identification, migration, and differentiation. The Anatomical Record 262, 1-15, doi:10.1002/1097-0185(20010101)262:1<1:AID-AR1006>3.0.CO; 2-2 (2001).
65 Prummel, K. D., Nieuwenhuize, S. & Mosimann, C. The lateral plate mesoderm. Development 147, dev175059, doi:10.1242/dev.175059 (2020).
66 Xue, R. et al. Expression of Cystic Fibrosis Transmembrane Conductance Regulator in Ganglia of Human Gastrointestinal Tract. Sci Rep 6, 30926, doi:10.1038/srep30926 (2016).
67 Morarach, K. et al. Diversification of molecularly defined myenteric neuron classes revealed by single cell RNA-sequencing. bioRxiv, 2020.2003.2002.955757, doi:10.1101/2020.03.02.955757 (2020).
68 May-Zhang, A. A. et al. Combinatorial Transcriptional Profiling of Mouse and Human Enteric Neurons Identifies Shared and Disparate Subtypes In Situ. Gastroenterology, doi:10.1053/j.gastro.2020.09.032 (2020).
69 Papaconstantinou, J. et al. Attenuation of p38alpha MAPK stress response signaling delays the in vivo aging of skeletal muscle myofibers and progenitor cells. Aging (Albany NY) 7, 718-733, doi:10.18632/aging.100802 (2015).
70 Soret, R. et al. Glial Cell-Derived Neurotrophic Factor Induces Enteric Neurogenesis and Improves Colon Structure and Function in Mouse Models of Hirschsprung Disease. Gastroenterology 159, 1824-1838 e1817, doi:10.1053/j.gastro.2020.07.018 (2020).
71 Kulkarni, S. et al. Adult enteric nervous system in health is maintained by a dynamic balance between neuronal apoptosis and neurogenesis. Proc Natl Acad Sci USA 114, E3709-E3718, doi:10.1073/pnas.1619406114 (2017).
72 Saffrey, M. J. Aging of the mammalian gastrointestinal tract: a complex organ system. Age (Dordr) 36, 9603, doi:10.1007/s11357-013-9603-2 (2014).
73 Barrenschee, M. et al. No neuronal loss, but alterations of the GDNF system in asymptomatic diverticulosis. PLoS One 12, e0171416, doi:10.1371/journal.pone.0171416 (2017).
74 Cossais, F. et al. Altered enteric expression of the homeobox transcription factor Phox2b in patients with diverticular disease. United European Gastroenterology Journal 7, 349-357, doi:10.1177/2050640618824913 (2019).
75 Schafmayer, C. et al. Genome-wide association analysis of diverticular disease points towards neuromuscular, connective tissue and epithelial pathomechanisms. Gut 68, 854, doi:10.1136/gutjnl-2018-317619 (2019).
76 Muller, P. A. et al. Microbiota-modulated CART(+) enteric neurons autonomously regulate blood glucose. Science 370, 314-321, doi:10.1126/science.abd6176 (2020).
77 Matheis, F. et al. Adrenergic Signaling in Muscularis Macrophages Limits Infection-Induced Neuronal Loss. Cell 180, 64-78 e16, doi:10.1016/j.cell.2019.12.002 (2020).
78 Becker, L., Kulkarni, S., Tiwari, G., Micci, M.-A. & Pasricha, P. J. Divergent fate and origin of neurosphere-like bodies from different layers of the gut. American Journal of Physiology—Gastrointestinal and Liver Physiology 302, G958-G965, doi:10.1152/ajpgi.00511.2011 (2012).
79 Becker, L., Peterson, J., Kulkarni, S. & Pasricha, P. J. Ex vivo neurogenesis within enteric ganglia occurs in a PTEN dependent manner. PLoS One 8, e59452 (2013).
80 Shenje, L. T. et al. Precardiac deletion of Numb and Numblike reveals renewal of cardiac progenitors. Elife 3, e02164, doi:10.7554/eLife.02164 (2014).
81 Susaki, E. A. et al. Whole-brain imaging with single-cell resolution using chemical cocktails and computational analysis. Cell 157, 726-739, doi:10.1016/j.cell.2014.03.042 (2014).
82 Bray, N. L., Pimentel, H., Melsted, P. & Pachter, L. Near-optimal probabilistic RNA-seq quantification. Nat Biotechnol 34, 525-527, doi:10.1038/nbt.3519 (2016).
83 Haghverdi, L., Lun, A. T. L., Morgan, M. D. & Marioni, J. C. Batch effects in single-cell RNA-sequencing data are corrected by matching mutual nearest neighbors. Nat Biotechnol 36, 421-427, doi:10.1038/nbt.4091 (2018).
84 Padovan-Merhar, O. et al. Single mammalian cells compensate for differences in cellular volume and DNA copy number through independent global transcriptional mechanisms. Mol Cell 58, 339-352, doi:10.1016/j.molcel.2015.03.005 (2015).
85 Gu, Z., Eils, R. & Schlesner, M. Complex heatmaps reveal patterns and correlations in multidimensional genomic data. Bioinformatics 32, 2847-2849, doi:10.1093/bioinformatics/btw313 (2016).
86 Tabula Muris, C. A single-cell transcriptomic atlas characterizes ageing tissues in the mouse. Nature 583, 590-595, doi:10.1038/s41586-020-2496-1 (2020).

### Example 2: Using GDNF as a Juvenile Protective Factor to Arrest and Reverse Aging in the Enteric Nervous System

This study demonstrates that the maturing and aging ENS exhibit remarkable developmental plasticity and that the fetal and the post-natal makeup of the ENS is significantly different. While the fetal ENS is indeed derived solely from the cells of the canonical embryonic derivation: Neural Crest (NC), the post-natal ENS contains populations of neurons derived from the mesoderm, whose proportional representations increases with maturity. The young adult ENS is made up of equal numbers of neurons from both lineages, which are important for normal ENS function.

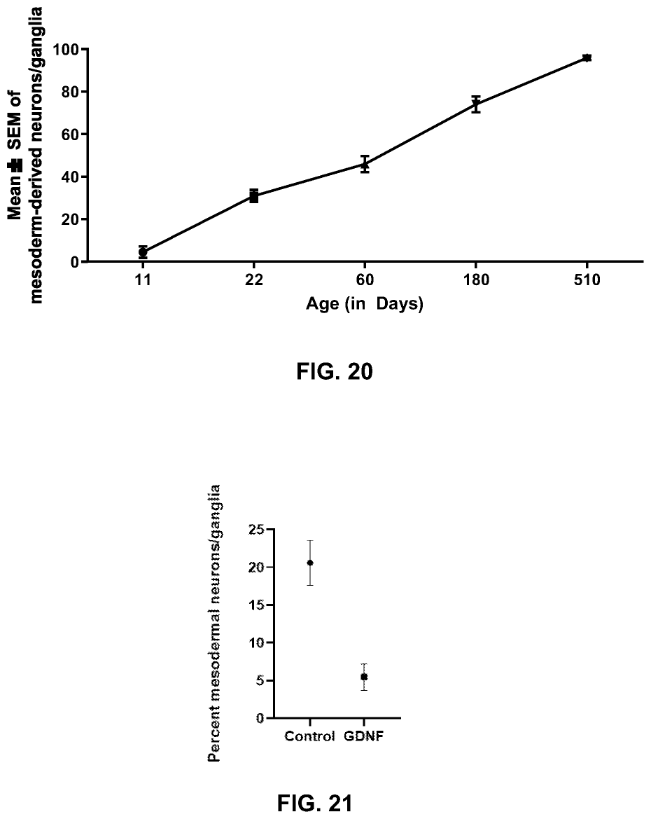

With aging, the neurons from the canonical NC lineage are progressively lost and are replaced by the neurons of the second lineage. In aging mice, the ENS is almost solely made-up of the neurons from the mesodermal lineage. This shift corresponds with age-associated decline in function (FIG. 20).

Reduced GDNF-RET signaling is associated with age-associated changes in the ENS: RET signaling is associated with promoting and maintaining neurogenesis of the canonical NC lineage in the ENS. Abrogated RET signaling in adults is associated with the onset of chronic intestinal dysmotility both in human patients as well as in mice models of reduced RET signaling. Glial Derived Neurotrophic Factor (GDNF) is a known agonist for RET which acts through the GFRA1 co-receptor to stimulate RET signaling (FIG. 21).

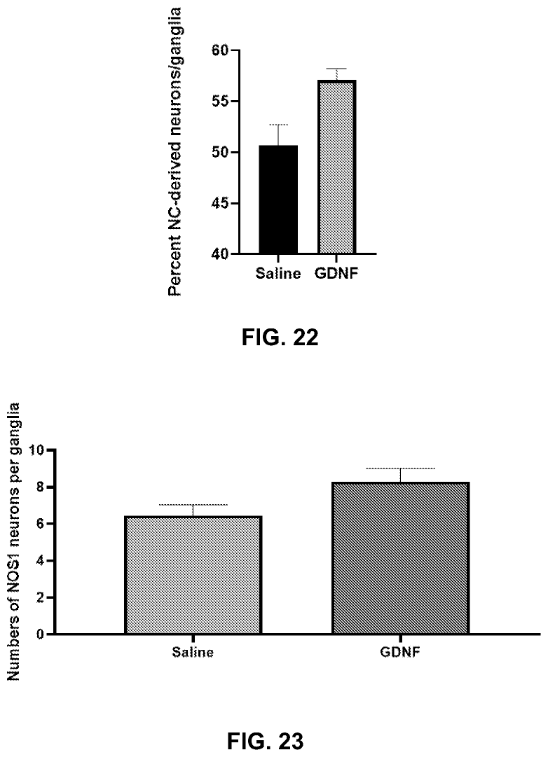

GDNF promotes maintenance of juvenile phenotype in the maturing ENS: Exogenous infusion of GDNF (100 μg subcutaneous) given thrice over a course of 10 days to juvenile (aged Post-natal Day 10) mice arrested their maturation-associated reduction in the numbers of canonical NC-derived enteric neurons (and a corresponding increase in the proportions of mesodermal-neurons), when compared to saline-treated controls. This shows that the exogenous addition of GDNF promotes an early Juvenile nature of the maturing ENS. Such factors have been termed as Juvenile Protective Factors (JPF). These physiological factors are intrinsic to a juvenile or immature organism, helping to maintain or enhance certain functions across all or some of the stages of development, but diminish or disappear during transition from one maturational stage to the next or at time of sexual maturity. Diminution or disappearance of JPFs after a given maturational stage or at time of sexual maturity could contribute to the onset of age-related declines in a variety of physiological functions (FIG. 22).

GDNF is a JPF that reverses aging in the adult ENS. Adult animals were similarly dosed with GDNF (1 μg subcutaneous; given 5 times over a course of 10 days), and found that the GDNF treated animals showed a significant reversal in their continual loss of NC neurons. The GDNF treated animals showed a significant increase in their proportions of canonical NC-derived enteric neurons over their saline treated control littermates, providing evidence that GDNF acts as an anti-aging factor that reverts age-associated developmental plasticity of enteric neurons. Finally, this GDNF-mediated shift also causes a significant increase in the genesis of NOS1-expressing neurons that are lost with aging and whose continued loss is associated with the incidence of intestinal dysmotility disorders. This provides evidence that the exogenous addition of GDNF to the aging and adult gut can reverse age-associated and disease-associated loss of NOS1-expressing neurons (FIG. 23).

The exogenous addition of GDNF is then the Juvenile Protective Factor that can be used as a therapeutic strategy for correcting age- and disease-associated pathological shifts in the normal and healthy proportions of the two lineages of the ENS. By promoting genesis and maintenance of the canonical NC-lineage that dominates during the juvenile phase of life, the increased presence of GDNF normalizes age and disease associated loss of this lineage and increases the genesis of NOS1-expressing neurons to normalize function.

### Example 3: The Representation of the Two Lineages of Neurons are Sex-Biased

The results obtained in determining whether the neuron lineages are the same in both males and females shows that in the young adult murine small intestine (2 month old or P60 mice), the relative proportions or representation of the two lineages of neurons is sex-biased, with males having a higher representation of the mesodermal neurons, compared to females. As the animals get older to the age where the reproductive capacity of female mice starts getting limited (6 month old or P180 mice), the representation of both neuronal lineages becomes similar.

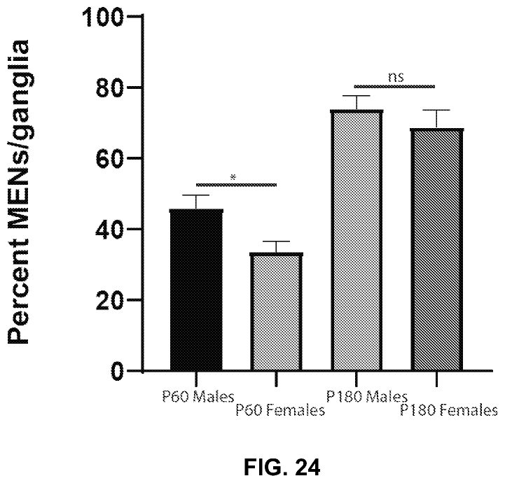

This provides evidence that sex-bias has a physiological and clinical relevance, diverse gut disorders that are associated with dysmotility occur with a very marked and robust female bias, where women account for about 80-90% of the patient pool. These also occur during the post-pubescent to menopausal period in women. The data in mice shows the significantly reduced proportions of mesodermal neurons in female mice of reproductive age when compared to age-matched male mice, a difference that is erased as females become reproductively limited, evidencing a strong causal effect between female-biased dysmotility and the proportions of the two neuronal lineages (FIG. 24).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating a subject with a gastrointestinal disorder, age or other disease associated structural alterations and neurodegeneration in the enteric nervous system comprising: administering to the subject a therapeutically effective amount of an agonist of RET (Rearranged during Transfection) receptor signaling and/or an antagonist of MET (hepatocyte growth factor receptor) receptor signaling, thereby treating the subject.

2. The method of claim 1, wherein the therapeutically effective amount of the agonist of RET receptor signaling increases the number of neural crest (NC)-derived enteric neuron (NENs) cells relative to Mesodermal-lineage of enteric neuron (MENs) cells.

3. (canceled)

4. The method of claim 1, wherein an agonist of RET signaling comprises: glial derived neurotrophic factor (GDNF), GDNF analogs, small molecules, peptide, oligonucleotides, antibodies, antibody fragments, single chain antibodies, antibody mimetics, peptoids, aptamers; enzymes, hormones, organic or inorganic molecules, natural or synthetic compounds.

5. The method of claim 2, wherein an NEN cell comprises one or more markers comprising: MHCST⁻ MET⁻, Ret, Uchll, Ncaml, Nosl, Plpl, Sl00b, RET, Soxl0, Snap25 and combinations thereof.

6. The method of claim 2, wherein a MEN cell comprises one or more markers comprising: MHCST⁺, Caleb (CGRP), Met, Cdh3, Slpi, Aebp1, Clic3, Fmo2, Smo, Myl7, Slcl7a9, Ntf3, I118 and combinations thereof.

7. A method of treating a treating a subject with a gastrointestinal disorder, comprising: administering to the subject a therapeutically effective amount of neural crest (NC)-derived enteric neuron (NENs) cells, thereby treating the subject.

9. The method of claim 8, wherein the NENs identified as being MHCST′ MET′.

10. The method of claim 9, wherein the isolated NENs are cultured in a medium comprising at least one agonist of RET receptor signaling.

11. The method of claim 10, wherein an agonist of RET signaling comprises: glial derived neurotrophic factor (GDNF), GDNF analogs, small molecules, peptide, polypeptides, oligonucleotides, antibodies, antibody fragments, single chain antibodies, antibody mimetics, peptoids, aptamers; enzymes, hormones, organic or inorganic molecules, natural or synthetic compounds.

12. The method of claim 11, wherein an agonist of RET signaling is GDNF, a GDNF analog, a GDNF mimic or the combination thereof.

13. (canceled)

14. The method of claim 7, further comprising administering one or more agonists of RET signaling.

15. The method of claim 7, further comprising administering one or more antagonist of MET receptor signaling.

16-28. (canceled)

29. A pharmaceutical composition comprising:
1) a therapeutically effective amount of one or more agonists of RET signaling; or
2) a therapeutically effective amount of one or more antagonists of MET receptor signaling; or
3) a therapeutically effective amount of one or more agonists of RET signaling and one or more antagonists of MET receptor signaling.

30-32. (canceled)

33. A method of correcting sex-biased lineage representation in a subject comprising: administering to the subject a therapeutically effective amount of an agonist of RET receptor signaling and/or an antagonist of MET receptor signaling, wherein the therapeutically effective amount of the agonist of RET receptor signaling increases the number of neural crest (NC)-derived enteric neuron (NENs) cells relative to Mesodermal-lineage of enteric neuron (MENs) cells, thereby correcting the sex biased lineage representation; or
A method of treating a subject with a gastrointestinal disorder, age or other disease associated with inflammatory bowel disease and associated intestinal dysmotility, comprising: administering to the subject a therapeutically effective amount of an antagonist of RET receptor signaling and/or an agonist of MET receptor signaling, thereby treating the subject; or
A method of treating a treating a subject with a gastrointestinal disorder, comprising: administering to the subject a therapeutically effective amount of mesoderm-derived enteric neuron (MENs) cells, thereby treating the subject.

34. The method of claim 33, wherein the subject is a female.

35. The method of claim 33, wherein an antagonist of MET receptor signaling decreases the number of MEN cells relative to NENs.

36. The method of claim 33, wherein an agonist of RET signaling comprises: glial derived neurotrophic factor (GDNF), GDNF analogs, small molecules, peptide, oligonucleotides, antibodies, antibody fragments, single chain antibodies, antibody mimetics, peptoids, aptamers; enzymes, hormones, organic or inorganic molecules, natural or synthetic compounds.

37. The method of claim 34, wherein an NEN cell comprises one or more markers comprising: MHCST⁻ MET⁻, Ret, Uchll, Ncam1, Nos1, Plp1, S100b, RET, Sox10, Snap25 and combinations thereof.

38. The method of claim 34, wherein a MEN cell comprises one or more markers comprising: MHCST⁺, Caleb (CGRP), Met, Cdh3, S1pi, Aebp1, Clic3, Fmo2, Smo, My17, S1c17a9, Ntf3, I118 and combinations thereof.

39. The method of claim 33, wherein the subject suffers from one or more gut disorders that are associated with dysmotility.

40-49. (canceled)
